(19) United States
(12) Patent Application Publication
(10) Pub. No.: US 2023/0279105 A1
(43) Pub. Date: Sep. 7, 2023

(54) ANTI-TIM-3 ANTIBODIES

(71) Applicant: Single Cell Technology, Inc., San Jose, CA (US)

(72) Inventors: Leyan TANG, San Jose, CA (US); Allison SCHULKINS, San Jose, CA (US); Kimberly THAN, San Jose, CA (US); Chun-Nan CHEN, San Jose, CA (US)

(73) Assignee: Single Cell Technology, Inc., San Jose, CA (US)

(21) Appl. No.: 17/621,509

(22) PCT Filed: Jun. 22, 2020
(86) PCT No.: PCT/US2020/038997

§ 371 Date: Dec. 21, 2021

Related U.S. Application Data
(60) Provisional application No. 62/864,835, filed on Jun. 21, 2019.

(51) Int. Cl. C07K 16/28

(52) U.S. Cl. CPC C07K 16/2803; C07K 2317/565

(57) ABSTRACT

Provided are antibodies that specifically recognize TIM-3 protein.

### CROSS-REFERENCE TO RELATED APPLICATIONS

This application is a U.S. National Phase Application under 35 U.S.C. § 371 of International Patent Application No. PCT/US2020/038997, filed on Jun. 22, 2020, which claims priority under 35 U.S.C. 119(e) to U.S. Provisional Application No. 62/864,835, filed Jun. 21, 2019, which is incorporated herein by reference in its entirety for all purposes the contents of each of which are hereby incorporated by reference into the present application in their entireties.

### INCORPORATION OF SEQUENCE LISTING

The instant application contains a Sequence Listing which has been submitted via EFS-Web and is hereby incorporated by reference in its entirety. The ASCII copy, created on Sep. 23, 2022, is named SCT0009-401-PC-Sequence_Listing_ST25.txt and is 409,600 bytes in size.

### BACKGROUND

T-cell immunoglobulin and mucin-domain containing-3 (TIM-3), also known as hepatitis A virus cellular receptor 2 (HAVCR2), is a type-I transmembrane protein that functions as a key regulator of immune responses. TIM-3 was initially identified on activated IFN-γ-producing T cells (e.g., type 1 helper CD4⁺ T cells and cytotoxic CD8⁺ T cells) and shown to induce T cell death or exhaustion after binding to galectin-9 (Monney et al., 2002). More recent studies have indicated that TIM-3 expression is also important in regulating the activities of many innate immune cells (e.g., macrophages, monocytes, dendritic cells, mast cells, and natural killer cells) (Han et al., 2013).

Like many inhibitory receptors (e.g., PD-1 and CTLA-4), TIM-3 expression has been associated with many types of chronic diseases, including cancer. TIM-3⁺ T cells have been detected in patients with advanced melanoma, non-small cell lung cancer, or follicular B-cell non-Hodgkin lymphoma. And the presence of TIM-3⁺ regulatory T cells have been described as an effective indicator of lung cancer progression (Anderson, 2014).

Several potential ligands for TIM-3 have been identified: Galectin-9, HMGB1, Semaphorin-4A, CEACAM-1, ILT-4 and phosphatidylserine (PtdSer or PS). PS is an important cell membrane component and is normally localized to the inner leaflet of cell membranes. But as a cell undergoes apoptosis, PS is redistributed and exposed to the outer membrane. This redistribution is also observed in many tumor cell lines (Riedl et al., 2011). Binding of TIM-3 to PS may be critical for phagocytosis and cross-presentation (Nakayama et al., 2009).

Studies have shown a close relationship between TIM-3 and the inhibitory receptor PD-1. For example, many tumor-specific T cells express both PD-1 and TIM-3, and these T cells have been shown to be more dysfunctional compared to T cells that express only PD-1 or TIM-3 (Fourcade et al., 2010).

Thus, the need exists for improved methods of treating cancer, such as therapeutic monoclonal antibodies. Medicines for use in such improved methods of treatment may comprise antibodies or antibody fragments that specifically bind to TIM-3 and reverse or partially reverse the TIM-3-mediated suppression of anti-tumor immune responses.

### SUMMARY

Provided are antibodies that recognize and specifically bind to human TIM-3. In one embodiment, the disclosure provides an antibody that binds to SEQ ID NO:1.

### BRIEF DESCRIPTION OF THE SEQUENCES

TABLE 1

| SEQ ID NO. | Description of sequence |
|---|---|
| 1 | Human TIM-3 protein sequence |
| 2 | Human TIM-3 His tagged protein sequence |
| 3 | Human TIM-3 Human Fc tagged protein sequence |
| 4 | SCT-Fa001 mature heavy chain variable domain protein sequence |
| 5 | SCT-Fa002 mature heavy chain variable domain protein sequence |
| 6 | SCT-Fa003 mature heavy chain variable domain protein sequence |
| 7 | SCT-Fa004 mature heavy chain variable domain protein sequence |
| 8 | SCT-Fa005 mature heavy chain variable domain protein sequence |
| 9 | SCT-Fa006 mature heavy chain variable domain protein sequence |
| 10 | SCT-Fa007 mature heavy chain variable domain protein sequence |
| 11 | SCT-Fa008 mature heavy chain variable domain protein sequence |
| 12 | SCT-Fa009 mature heavy chain variable domain protein sequence |
| 13 | SCT-Fa010 mature heavy chain variable domain protein sequence |
| 14 | SCT-Fa011 mature heavy chain variable domain protein sequence |
| 14 | SCT-Fa012 mature heavy chain variable domain protein sequence |
| 15 | SCT-Fa013 mature heavy chain variable domain protein sequence |
| 15 | SCT-Fa014 mature heavy chain variable domain protein sequence |
| 16 | SCT-Fa015 mature heavy chain variable domain protein sequence |
| 17 | SCT-Fa016 mature heavy chain variable domain protein sequence |
| 18 | SCT-Fa017 mature heavy chain variable domain protein sequence |
| 19 | SCT-Fa018 mature heavy chain variable domain protein sequence |
| 20 | SCT-Fa019 mature heavy chain variable domain protein sequence |
| 21 | SCT-Fa020 mature heavy chain variable domain protein sequence |
| 22 | SCT-Fa021 mature heavy chain variable domain protein sequence |
| 23 | SCT-Fa022 mature heavy chain variable domain protein sequence |
| 24 | SCT-Fa023 mature heavy chain variable domain protein sequence |
| 25 | SCT-Fa024 mature heavy chain variable domain protein sequence |
| 26 | SCT-Fa025 mature heavy chain variable domain protein sequence |
| 27 | SCT-Fa026 mature heavy chain variable domain protein sequence |
| 28 | SCT-Fa027 mature heavy chain variable domain protein sequence |
| 29 | SCT-Fa028 mature heavy chain variable domain protein sequence |
| 30 | SCT-Fa029 mature heavy chain variable domain protein sequence |
| 31 | SCT-Fa030 mature heavy chain variable domain protein sequence |
| 32 | SCT-Fa031 mature heavy chain variable domain protein sequence |
| 33 | SCT-Fa032 mature heavy chain variable domain protein sequence |
| 34 | SCT-Fa033 mature heavy chain variable domain protein sequence |
| 35 | SCT-Fa034 mature heavy chain variable domain protein sequence |
| 35 | SCT-Fa035 mature heavy chain variable domain protein sequence |
| 36 | SCT-Fa036 mature heavy chain variable domain protein sequence |
| 37 | SCT-Fa037 mature heavy chain variable domain protein sequence |
| 38 | SCT-Fa038 mature heavy chain variable domain protein sequence |
| 39 | SCT-Fa039 mature heavy chain variable domain protein sequence |
| 40 | SCT-Fa040 mature heavy chain variable domain protein sequence |
| 41 | SCT-Fa041 mature heavy chain variable domain protein sequence |
| 42 | SCT-Fa042 mature heavy chain variable domain protein sequence |
| 43 | SCT-Fa043 mature heavy chain variable domain protein sequence |
| 44 | SCT-Fa044 mature heavy chain variable domain protein sequence |
| 45 | SCT-Fa045 mature heavy chain variable domain protein sequence |
| 46 | SCT-Fa046 mature heavy chain variable domain protein sequence |
| 47 | SCT-Fa047 mature heavy chain variable domain protein sequence |
| 48 | SCT-Fa048 mature heavy chain variable domain protein sequence |
| 49 | SCT-Fa049 mature heavy chain variable domain protein sequence |
| 50 | SCT-Fa050 mature heavy chain variable domain protein sequence |
| 51 | SCT-Fa051 mature heavy chain variable domain protein sequence |
| 52 | SCT-Fa052 mature heavy chain variable domain protein sequence |
| 53 | SCT-Fa053 mature heavy chain variable domain protein sequence |
| 54 | SCT-Fa054 mature heavy chain variable domain protein sequence |
| 55 | SCT-Fa055 mature heavy chain variable domain protein sequence |
| 56 | SCT-Fa056 mature heavy chain variable domain protein sequence |
| 57 | SCT-Fa057 mature heavy chain variable domain protein sequence |
| 58 | SCT-Fa058 mature heavy chain variable domain protein sequence |
| 59 | SCT-Fa059 mature heavy chain variable domain protein sequence |
| 60 | SCT-Fa060 mature heavy chain variable domain protein sequence |
| 61 | SCT-Fa061 mature heavy chain variable domain protein sequence |
| 62 | SCT-Fa062 mature heavy chain variable domain protein sequence |
| 63 | SCT-Fa063 mature heavy chain variable domain protein sequence |
| 64 | SCT-Fa064 mature heavy chain variable domain protein sequence |
| 65 | SCT-Fa065 mature heavy chain variable domain protein sequence |
| 66 | SCT-Fa066 mature heavy chain variable domain protein sequence |
| 67 | SCT-Fa067 mature heavy chain variable domain protein sequence |
| 67 | SCT-Fa068 mature heavy chain variable domain protein sequence |
| 68 | SCT-Fa069 mature heavy chain variable domain protein sequence |
| 69 | SCT-Fa070 mature heavy chain variable domain protein sequence |
| 70 | SCT-Fa071 mature heavy chain variable domain protein sequence |
| 71 | SCT-Fa072 mature heavy chain variable domain protein sequence |
| 72 | SCT-Fa073 mature heavy chain variable domain protein sequence |
| 73 | SCT-Fa074 mature heavy chain variable domain protein sequence |
| 74 | SCT-Fa075 mature heavy chain variable domain protein sequence |
| 75 | SCT-Fa076 mature heavy chain variable domain protein sequence |
| 76 | SCT-Fa077 mature heavy chain variable domain protein sequence |
| 77 | SCT-Fa078 mature heavy chain variable domain protein sequence |
| 78 | SCT-Fa079 mature heavy chain variable domain protein sequence |
| 79 | SCT-Fa080 mature heavy chain variable domain protein sequence |
| 80 | SCT-Fa081 mature heavy chain variable domain protein sequence |
| 81 | SCT-Fa082 mature heavy chain variable domain protein sequence |
| 82 | SCT-Fa083 mature heavy chain variable domain protein sequence |
| 83 | SCT-Fa084 mature heavy chain variable domain protein sequence |
| 84 | SCT-Fa085 mature heavy chain variable domain protein sequence |
| 85 | SCT-Fa086 mature heavy chain variable domain protein sequence |
| 86 | SCT-Fa087 mature heavy chain variable domain protein sequence |
| 87 | SCT-Fa088 mature heavy chain variable domain protein sequence |
| 88 | SCT-Fa089 mature heavy chain variable domain protein sequence |
| 89 | SCT-Fa090 mature heavy chain variable domain protein sequence |
| 90 | SCT-Fa091 mature heavy chain variable domain protein sequence |
| 90 | SCT-Fa092 mature heavy chain variable domain protein sequence |
| 91 | SCT-Fa093 mature heavy chain variable domain protein sequence |
| 92 | SCT-Fa094 mature heavy chain variable domain protein sequence |
| 93 | SCT-Fa095 mature heavy chain variable domain protein sequence |
| 94 | SCT-Fa096 mature heavy chain variable domain protein sequence |
| 95 | SCT-Fa097 mature heavy chain variable domain protein sequence |
| 96 | SCT-Fa098 mature heavy chain variable domain protein sequence |
| 96 | SCT-Fa099 mature heavy chain variable domain protein sequence |
| 97 | SCT-Fa100 mature heavy chain variable domain protein sequence |
| 98 | SCT-Fa101 mature heavy chain variable domain protein sequence |
| 99 | SCT-Fa102 mature heavy chain variable domain protein sequence |
| 100 | SCT-Fa103 mature heavy chain variable domain protein sequence |
| 101 | SCT-Fa104 mature heavy chain variable domain protein sequence |
| 102 | SCT-Fa105 mature heavy chain variable domain protein sequence |
| 103 | SCT-Fa106 mature heavy chain variable domain protein sequence |
| 104 | SCT-Fa107 mature heavy chain variable domain protein sequence |
| 105 | SCT-Fa108 mature heavy chain variable domain protein sequence |
| 106 | SCT-Fa109 mature heavy chain variable domain protein sequence |
| 107 | SCT-Fa110 mature heavy chain variable domain protein sequence |
| 108 | SCT-Fa111 mature heavy chain variable domain protein sequence |
| 109 | SCT-Fa112 mature heavy chain variable domain protein sequence |
| 110 | SCT-Fa113 mature heavy chain variable domain protein sequence |
| 111 | SCT-Fa114 mature heavy chain variable domain protein sequence |
| 112 | SCT-Fa115 mature heavy chain variable domain protein sequence |
| 113 | SCT-Fa116 mature heavy chain variable domain protein sequence |
| 114 | SCT-Fa117 mature heavy chain variable domain protein sequence |
| 115 | SCT-Fa118 mature heavy chain variable domain protein sequence |
| 116 | SCT-Fa119 mature heavy chain variable domain protein sequence |
| 117 | SCT-Fa120 mature heavy chain variable domain protein sequence |
| 118 | SCT-Fa121 mature heavy chain variable domain protein sequence |
| 119 | SCT-Fa122 mature heavy chain variable domain protein sequence |
| 120 | SCT-Fa123 mature heavy chain variable domain protein sequence |
| 121 | SCT-Fa124 mature heavy chain variable domain protein sequence |
| 122 | SCT-Fa125 mature heavy chain variable domain protein sequence |
| 123 | SCT-Fa126 mature heavy chain variable domain protein sequence |
| 124 | SCT-Fa127 mature heavy chain variable domain protein sequence |
| 125 | SCT-Fa128 mature heavy chain variable domain protein sequence |
| 126 | SCT-Fa129 mature heavy chain variable domain protein sequence |
| 127 | SCT-Fa130 mature heavy chain variable domain protein sequence |
| 128 | SCT-Fa131 mature heavy chain variable domain protein sequence |
| 129 | SCT-Fa132 mature heavy chain variable domain protein sequence |
| 130 | SCT-Fa133 mature heavy chain variable domain protein sequence |
| 131 | SCT-Fa134 mature heavy chain variable domain protein sequence |
| 132 | SCT-Fa135 mature heavy chain variable domain protein sequence |
| 133 | SCT-Fa136 mature heavy chain variable domain protein sequence |
| 134 | SCT-Fa137 mature heavy chain variable domain protein sequence |
| 135 | SCT-Fa138 mature heavy chain variable domain protein sequence |
| 136 | SCT-Fa139 mature heavy chain variable domain protein sequence |
| 137 | SCT-Fa140 mature heavy chain variable domain protein sequence |
| 138 | SCT-Fa141 mature heavy chain variable domain protein sequence |
| 139 | SCT-Fa142 mature heavy chain variable domain protein sequence |
| 140 | SCT-Fa143 mature heavy chain variable domain protein sequence |
| 141 | SCT-Fa144 mature heavy chain variable domain protein sequence |
| 142 | SCT-Fa145 mature heavy chain variable domain protein sequence |
| 143 | SCT-Fa146 mature heavy chain variable domain protein sequence |
| 144 | SCT-Fa147 mature heavy chain variable domain protein sequence |
| 145 | SCT-Fa148 mature heavy chain variable domain protein sequence |
| 146 | SCT-Fa149 mature heavy chain variable domain protein sequence |
| 147 | SCT-Fa150 mature heavy chain variable domain protein sequence |
| 148 | SCT-Fa151 mature heavy chain variable domain protein sequence |
| 149 | SCT-Fa152 mature heavy chain variable domain protein sequence |
| 150 | SCT-Fa153 mature heavy chain variable domain protein sequence |
| 151 | SCT-Fa154 mature heavy chain variable domain protein sequence |
| 152 | SCT-Fa155 mature heavy chain variable domain protein sequence |
| 153 | SCT-Fa156 mature heavy chain variable domain protein sequence |
| 154 | SCT-Fa157 mature heavy chain variable domain protein sequence |
| 155 | SCT-Fa158 mature heavy chain variable domain protein sequence |
| 156 | SCT-Fa159 mature heavy chain variable domain protein sequence |
| 156 | SCT-Fa160 mature heavy chain variable domain protein sequence |
| 156 | SCT-Fa161 mature heavy chain variable domain protein sequence |
| 157 | SCT-Fa162 mature heavy chain variable domain protein sequence |
| 157 | SCT-Fa163 mature heavy chain variable domain protein sequence |
| 158 | SCT-Fa164 mature heavy chain variable domain protein sequence |
| 159 | SCT-Fa165 mature heavy chain variable domain protein sequence |
| 160 | SCT-Fa166 mature heavy chain variable domain protein sequence |
| 161 | SCT-Fa167 mature heavy chain variable domain protein sequence |
| 162 | SCT-Fa168 mature heavy chain variable domain protein sequence |
| 163 | SCT-Fa169 mature heavy chain variable domain protein sequence |
| 164 | SCT-Fa170 mature heavy chain variable domain protein sequence |
| 165 | SCT-Fa171 mature heavy chain variable domain protein sequence |
| 166 | SCT-Fa172 mature heavy chain variable domain protein sequence |
| 167 | SCT-Fa173 mature heavy chain variable domain protein sequence |
| 168 | SCT-Fa174 mature heavy chain variable domain protein sequence |
| 169 | SCT-Fa175 mature heavy chain variable domain protein sequence |
| 170 | SCT-Fa176 mature heavy chain variable domain protein sequence |
| 171 | SCT-Fa177 mature heavy chain variable domain protein sequence |
| 171 | SCT-Fa178 mature heavy chain variable domain protein sequence |
| 172 | SCT-Fa179 mature heavy chain variable domain protein sequence |
| 173 | SCT-Fa180 mature heavy chain variable domain protein sequence |
| 174 | SCT-Fa181 mature heavy chain variable domain protein sequence |
| 175 | SCT-Fa182 mature heavy chain variable domain protein sequence |
| 175 | SCT-Fa183 mature heavy chain variable domain protein sequence |
| 176 | SCT-Fa184 mature heavy chain variable domain protein sequence |
| 177 | SCT-Fa185 mature heavy chain variable domain protein sequence |
| 178 | SCT-Fa186 mature heavy chain variable domain protein sequence |
| 179 | SCT-Fa187 mature heavy chain variable domain protein sequence |
| 180 | SCT-Fa188 mature heavy chain variable domain protein sequence |
| 181 | SCT-Fa189 mature heavy chain variable domain protein sequence |
| 182 | SCT-Fa190 mature heavy chain variable domain protein sequence |
| 183 | SCT-Fa191 mature heavy chain variable domain protein sequence |
| 184 | SCT-Fa192 mature heavy chain variable domain protein sequence |
| 185 | SCT-Fa193 mature heavy chain variable domain protein sequence |
| 186 | SCT-Fa194 mature heavy chain variable domain protein sequence |
| 186 | SCT-Fa195 mature heavy chain variable domain protein sequence |
| 187 | SCT-Fa118 mature light chain variable domain protein sequence |
| 188 | SCT-Fa159 mature light chain variable domain protein sequence |
| 189 | SCT-Fa160 mature light chain variable domain protein sequence |
| 190 | SCT-Fa152 mature light chain variable domain protein sequence |
| 191 | SCT-Fa165 mature light chain variable domain protein sequence |
| 191 | SCT-Fa166 mature light chain variable domain protein sequence |
| 192 | SCT-Fa158 mature light chain variable domain protein sequence |
| 192 | SCT-Fa162 mature light chain variable domain protein sequence |
| 192 | SCT-Fa164 mature light chain variable domain protein sequence |
| 192 | SCT-Fa167 mature light chain variable domain protein sequence |
| 192 | SCT-Fa168 mature light chain variable domain protein sequence |
| 193 | SCT-Fa153 mature light chain variable domain protein sequence |
| 194 | SCT-Fa154 mature light chain variable domain protein sequence |
| 194 | SCT-Fa156 mature light chain variable domain protein sequence |
| 195 | SCT-Fa139 mature light chain variable domain protein sequence |
| 196 | SCT-Fa184 mature light chain variable domain protein sequence |
| 197 | SCT-Fa091 mature light chain variable domain protein sequence |
| 198 | SCT-Fa090 mature light chain variable domain protein sequence |
| 198 | SCT-Fa092 mature light chain variable domain protein sequence |
| 199 | SCT-Fa182 mature light chain variable domain protein sequence |
| 200 | SCT-Fa180 mature light chain variable domain protein sequence |
| 201 | SCT-Fa185 mature light chain variable domain protein sequence |
| 202 | SCT-Fa055 mature light chain variable domain protein sequence |
| 203 | SCT-Fa061 mature light chain variable domain protein sequence |
| 204 | SCT-Fa054 mature light chain variable domain protein sequence |
| 205 | SCT-Fa053 mature light chain variable domain protein sequence |
| 206 | SCT-Fa052 mature light chain variable domain protein sequence |
| 207 | SCT-Fa179 mature light chain variable domain protein sequence |
| 208 | SCT-Fa189 mature light chain variable domain protein sequence |
| 209 | SCT-Fa174 mature light chain variable domain protein sequence |
| 209 | SCT-Fa187 mature light chain variable domain protein sequence |
| 209 | SCT-Fa188 mature light chain variable domain protein sequence |
| 210 | SCT-Fa183 mature light chain variable domain protein sequence |
| 211 | SCT-Fa002 mature light chain variable domain protein sequence |
| 211 | SCT-Fa079 mature light chain variable domain protein sequence |
| 211 | SCT-Fa146 mature light chain variable domain protein sequence |
| 212 | SCT-Fa113 mature light chain variable domain protein sequence |
| 213 | SCT-Fa083 mature light chain variable domain protein sequence |
| 214 | SCT-Fa078 mature light chain variable domain protein sequence |
| 214 | SCT-Fa080 mature light chain variable domain protein sequence |
| 214 | SCT-Fa081 mature light chain variable domain protein sequence |
| 214 | SCT-Fa082 mature light chain variable domain protein sequence |
| 214 | SCT-Fa112 mature light chain variable domain protein sequence |
| 214 | SCT-Fa145 mature light chain variable domain protein sequence |
| 214 | SCT-Fa147 mature light chain variable domain protein sequence |
| 215 | SCT-Fa084 mature light chain variable domain protein sequence |
| 216 | SCT-Fa149 mature light chain variable domain protein sequence |
| 217 | SCT-Fa077 mature light chain variable domain protein sequence |
| 218 | SCT-Fa148 mature light chain variable domain protein sequence |
| 219 | SCT-Fa128 mature light chain variable domain protein sequence |
| 219 | SCT-Fa129 mature light chain variable domain protein sequence |
| 220 | SCT-Fa032 mature light chain variable domain protein sequence |
| 221 | SCT-Fa033 mature light chain variable domain protein sequence |
| 222 | SCT-Fa097 mature light chain variable domain protein sequence |
| 223 | SCT-Fa004 mature light chain variable domain protein sequence |
| 224 | SCT-Fa029 mature light chain variable domain protein sequence |
| 225 | SCT-Fa105 mature light chain variable domain protein sequence |
| 226 | SCT-Fa194 mature light chain variable domain protein sequence |
| 227 | SCT-Fa098 mature light chain variable domain protein sequence |
| 228 | SCT-Fa039 mature light chain variable domain protein sequence |
| 228 | SCT-Fa040 mature light chain variable domain protein sequence |
| 229 | SCT-Fa093 mature light chain variable domain protein sequence |
| 229 | SCT-Fa099 mature light chain variable domain protein sequence |
| 229 | SCT-Fa100 mature light chain variable domain protein sequence |
| 229 | SCT-Fa101 mature light chain variable domain protein sequence |
| 229 | SCT-Fa102 mature light chain variable domain protein sequence |
| 229 | SCT-Fa103 mature light chain variable domain protein sequence |
| 229 | SCT-Fa107 mature light chain variable domain protein sequence |
| 229 | SCT-Fa110 mature light chain variable domain protein sequence |
| 230 | SCT-Fa104 mature light chain variable domain protein sequence |
| 230 | SCT-Fa108 mature light chain variable domain protein sequence |
| 230 | SCT-Fa109 mature light chain variable domain protein sequence |
| 231 | SCT-Fa027 mature light chain variable domain protein sequence |
| 231 | SCT-Fa041 mature light chain variable domain protein sequence |
| 232 | SCT-Fa028 mature light chain variable domain protein sequence |
| 232 | SCT-Fa042 mature light chain variable domain protein sequence |
| 233 | SCT-Fa043 mature light chain variable domain protein sequence |
| 233 | SCT-Fa044 mature light chain variable domain protein sequence |
| 234 | SCT-Fa106 mature light chain variable domain protein sequence |
| 235 | SCT-Fa030 mature light chain variable domain protein sequence |
| 236 | SCT-Fa025 mature light chain variable domain protein sequence |
| 236 | SCT-Fa026 mature light chain variable domain protein sequence |
| 236 | SCT-Fa038 mature light chain variable domain protein sequence |
| 237 | SCT-Fa023 mature light chain variable domain protein sequence |
| 238 | SCT-Fa024 mature light chain variable domain protein sequence |
| 239 | SCT-Fa037 mature light chain variable domain protein sequence |
| 240 | SCT-Fa031 mature light chain variable domain protein sequence |
| 240 | SCT-Fa034 mature light chain variable domain protein sequence |
| 241 | SCT-Fa009 mature light chain variable domain protein sequence |
| 242 | SCT-Fa163 mature light chain variable domain protein sequence |
| 243 | SCT-Fa076 mature light chain variable domain protein sequence |
| 244 | SCT-Fa133 mature light chain variable domain protein sequence |
| 245 | SCT-Fa131 mature light chain variable domain protein sequence |
| 246 | SCT-Fa020 mature light chain variable domain protein sequence |
| 247 | SCT-Fa181 mature light chain variable domain protein sequence |
| 248 | SCT-Fa169 mature light chain variable domain protein sequence |
| 249 | SCT-Fa124 mature light chain variable domain protein sequence |
| 250 | SCT-Fa122 mature light chain variable domain protein sequence |
| 251 | SCT-Fa130 mature light chain variable domain protein sequence |
| 252 | SCT-Fa126 mature light chain variable domain protein sequence |
| 253 | SCT-Fa123 mature light chain variable domain protein sequence |
| 253 | SCT-Fa190 mature light chain variable domain protein sequence |
| 254 | SCT-Fa132 mature light chain variable domain protein sequence |
| 255 | SCT-Fa144 mature light chain variable domain protein sequence |
| 256 | SCT-Fa125 mature light chain variable domain protein sequence |
| 257 | SCT-Fa127 mature light chain variable domain protein sequence |
| 258 | SCT-Fa195 mature light chain variable domain protein sequence |
| 259 | SCT-Fa059 mature light chain variable domain protein sequence |
| 260 | SCT-Fa005 mature light chain variable domain protein sequence |
| 261 | SCT-Fa021 mature light chain variable domain protein sequence |
| 262 | SCT-Fa051 mature light chain variable domain protein sequence |
| 263 | SCT-Fa056 mature light chain variable domain protein sequence |
| 264 | SCT-Fa071 mature light chain variable domain protein sequence |
| 265 | SCT-Fa064 mature light chain variable domain protein sequence |
| 266 | SCT-Fa115 mature light chain variable domain protein sequence |
| 266 | SCT-Fa117 mature light chain variable domain protein sequence |
| 267 | SCT-Fa096 mature light chain variable domain protein sequence |
| 268 | SCT-Fa114 mature light chain variable domain protein sequence |
| 269 | SCT-Fa045 mature light chain variable domain protein sequence |
| 270 | SCT-Fa170 mature light chain variable domain protein sequence |
| 271 | SCT-Fa135 mature light chain variable domain protein sequence |
| 272 | SCT-Fa134 mature light chain variable domain protein sequence |
| 273 | SCT-Fa171 mature light chain variable domain protein sequence |
| 274 | SCT-Fa186 mature light chain variable domain protein sequence |
| 275 | SCT-Fa192 mature light chain variable domain protein sequence |
| 276 | SCT-Fa191 mature light chain variable domain protein sequence |
| 277 | SCT-Fa036 mature light chain variable domain protein sequence |
| 278 | SCT-Fa121 mature light chain variable domain protein sequence |
| 279 | SCT-Fa176 mature light chain variable domain protein sequence |
| 280 | SCT-Fa116 mature light chain variable domain protein sequence |
| 280 | SCT-Fa119 mature light chain variable domain protein sequence |
| 281 | SCT-Fa120 mature light chain variable domain protein sequence |
| 281 | SCT-Fa172 mature light chain variable domain protein sequence |
| 282 | SCT-Fa111 mature light chain variable domain protein sequence |
| 283 | SCT-Fa048 mature light chain variable domain protein sequence |
| 283 | SCT-Fa049 mature light chain variable domain protein sequence |
| 284 | SCT-Fa047 mature light chain variable domain protein sequence |
| 285 | SCT-Fa095 mature light chain variable domain protein sequence |
| 286 | SCT-Fa150 mature light chain variable domain protein sequence |
| 287 | SCT-Fa060 mature light chain variable domain protein sequence |
| 288 | SCT-Fa016 mature light chain variable domain protein sequence |
| 289 | SCT-Fa136 mature light chain variable domain protein sequence |
| 290 | SCT-Fa067 mature light chain variable domain protein sequence |
| 291 | SCT-Fa140 mature light chain variable domain protein sequence |
| 292 | SCT-Fa138 mature light chain variable domain protein sequence |
| 293 | SCT-Fa193 mature light chain variable domain protein sequence |
| 294 | SCT-Fa143 mature light chain variable domain protein sequence |
| 295 | SCT-Fa151 mature light chain variable domain protein sequence |
| 296 | SCT-Fa142 mature light chain variable domain protein sequence |
| 297 | SCT-Fa155 mature light chain variable domain protein sequence |
| 297 | SCT-Fa157 mature light chain variable domain protein sequence |
| 298 | SCT-Fa018 mature light chain variable domain protein sequence |
| 299 | SCT-Fa094 mature light chain variable domain protein sequence |
| 300 | SCT-Fa072 mature light chain variable domain protein sequence |
| 301 | SCT-Fa137 mature light chain variable domain protein sequence |
| 302 | SCT-Fa003 mature light chain variable domain protein sequence |
| 303 | SCT-Fa177 mature light chain variable domain protein sequence |
| 304 | SCT-Fa141 mature light chain variable domain protein sequence |
| 305 | SCT-Fa007 mature light chain variable domain protein sequence |
| 305 | SCT-Fa046 mature light chain variable domain protein sequence |
| 306 | SCT-Fa010 mature light chain variable domain protein sequence |
| 307 | SCT-Fa019 mature light chain variable domain protein sequence |
| 308 | SCT-Fa066 mature light chain variable domain protein sequence |
| 309 | SCT-Fa069 mature light chain variable domain protein sequence |
| 310 | SCT-Fa057 mature light chain variable domain protein sequence |
| 310 | SCT-Fa062 mature light chain variable domain protein sequence |
| 310 | SCT-Fa065 mature light chain variable domain protein sequence |
| 310 | SCT-Fa068 mature light chain variable domain protein sequence |
| 310 | SCT-Fa070 mature light chain variable domain protein sequence |
| 311 | SCT-Fa063 mature light chain variable domain protein sequence |
| 312 | SCT-Fa013 mature light chain variable domain protein sequence |
| 313 | SCT-Fa017 mature light chain variable domain protein sequence |
| 314 | SCT-Fa014 mature light chain variable domain protein sequence |
| 314 | SCT-Fa015 mature light chain variable domain protein sequence |
| 315 | SCT-Fa011 mature light chain variable domain protein sequence |
| 316 | SCT-Fa012 mature light chain variable domain protein sequence |
| 317 | SCT-Fa001 mature light chain variable domain protein sequence |
| 317 | SCT-Fa035 mature light chain variable domain protein sequence |
| 318 | SCT-Fa050 mature light chain variable domain protein sequence |
| 319 | SCT-Fa008 mature light chain variable domain protein sequence |
| 320 | SCT-Fa058 mature light chain variable domain protein sequence |
| 321 | SCT-Fa175 mature light chain variable domain protein sequence |
| 322 | SCT-Fa173 mature light chain variable domain protein sequence |
| 323 | SCT-Fa178 mature light chain variable domain protein sequence |
| 324 | SCT-Fa073 mature light chain variable domain protein sequence |
| 325 | SCT-Fa006 mature light chain variable domain protein sequence |
| 326 | SCT-Fa074 mature light chain variable domain protein sequence |
| 326 | SCT-Fa075 mature light chain variable domain protein sequence |
| 327 | SCT-Fa161 mature light chain variable domain protein sequence |
| 328 | SCT-Fa086 mature light chain variable domain protein sequence |
| 329 | SCT-Fa088 mature light chain variable domain protein sequence |
| 330 | SCT-Fa022 mature light chain variable domain protein sequence |
| 331 | SCT-Fa087 mature light chain variable domain protein sequence |
| 332 | SCT-Fa085 mature light chain variable domain protein sequence |
| 332 | SCT-Fa089 mature light chain variable domain protein sequence |

### DETAILED DESCRIPTION

The disclosure provides antibodies that bind to TIM-3. In one embodiment, the disclosure provides an isolated antibody that binds to SEQ ID NO: 1.

### Antibodies

The disclosure provides antibodies that bind specifically to SEQ ID NO:1. The term “antibody” as used herein, includes both full-length immunoglobulins and antibody fragments that bind to the same antigens. The antibodies can be, e.g., a monoclonal, polyclonal, chimeric, humanized, or single chain antibody. As used herein, the terms “antigen binding fragment,” “fragment,” and “antibody fragment” are used interchangeably to refer to any fragment that comprises a portion of a full-length antibody, generally at least the antigen binding portion or the variable region thereof. Examples of antibody fragments include, but are not limited to, diabodies, single-chain antibody molecules, multi-specific antibodies, Fab, Fab′, F(ab′)2, Fv or scFv.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The terms “subject” and “patient” are used interchangeably herein to mean all mammals including humans. Examples of subjects include, but are not limited to, humans, monkeys, dogs, cats, horses, cows, goats, sheep, pigs, and rabbits. In one embodiment, the subject or patient is a human.

In certain embodiments, the cancer treated with the anti-TIM-3 antibody molecule, alone or in combination, includes but is not limited to, a solid tumor, a hematological cancer (e.g., leukemia, lymphoma, myeloma), and a metastatic lesion thereof. In one embodiment, the cancer is a solid tumor. Examples of solid tumors include malignancies, e.g., sarcomas and carcinomas (e.g., adenocarcinomas), of the various organ systems, such as those affecting lung, breast, lymphoid, gastrointestinal or colorectal, genitals and genitourinary tract (e.g., renal, urothelial, bladder cells), pharynx, CNS (e.g., brain, neural or glial cells), skin (e.g., melanoma), head and neck (e.g., head and neck squamous cell carcinoma (HNCC)), and pancreas. For example, melanoma, colon cancers, gastric cancer, rectal cancer, renal-cell carcinoma, breast cancer (e.g., a breast cancer that does not express one, two or all of estrogen receptor, progesterone receptor, or Her2/neu, e.g., a triple negative breast cancer), liver cancer, a lung cancer (e.g., a non-small cell lung cancer (NSCLC) (e.g., a NSCLC with squamous and/or non-squamous histology) or small cell lung cancer), prostate cancer, cancer of head or neck (e.g., HPV+ squamous cell carcinoma), cancer of the small intestine and cancer of the esophagus. Examples of hematological cancer include, but is not limited to, leukemia (e.g., a myeloid leukemia, lymphoid leukemia, or chronic lymphocytic leukemia (CLL)), lymphoma (e.g., Hogdkin lymphoma (HL), non-Hogdkin lymphoma (NHL), Diffuse large B-cell lymphoma (DLBCL), T-cell lymphoma, or mantle cell lymphoma (MCL)), and myeloma, e.g., multiple myeloma. The cancer may be at an early, intermediate, late stage or metastatic cancer. For discovering and developing antibody therapeutics to TIM-3 generally known in the art, please see U.S. Pat. No. 10,077,306, which is incorporated in its entirety by reference.

The provided antibodies disclosed herein can be used to treat, prevent and/or diagnose cancerous or malignant disorders (e.g., cancers such melanoma, e.g., advanced stage melanoma; pancreatic cancer, e.g., advanced pancreatic cancer; solid tumors; breast cancer, e.g., metastatic breast carcinoma; renal cell carcinoma, e.g., advanced or metastatic renal cell carcinoma (MRCC) or clear cell renal cell carcinoma).

The provided antibodies may be used to diagnose, treat, or monitor TIM-3-expressing cancer and its progression, regression, or stability; to determine whether or not a patient should be treated for cancer; or to determine whether or not a subject is afflicted with TIM-3-expressing cancer and thus may be amenable to treatment with a TIM-3-specific anticancer therapeutic. In some embodiments, the antibodies or fragments thereof described herein may be used for various in vitro molecular-biology applications such as, for example, enzyme-linked immunosorbent assays (ELISA), Western blots, immunohistochemistry, immunocytochemistry, flow cytometry and fluorescence-activated cell sorting (FACS), immunoprecipitation, and/or enzyme-linked immunospot assays (ELISPOT). In some embodiments, the antibodies or fragments thereof may be packaged in kits with or without additional reagents known to those of skill in the art for practicing any of the molecular biology techniques disclosed above.

The disclosure provides the antibodies SCT-Fa001, SCT-Fa002, SCT-Fa003, SCT-Fa004, SCT-Fa005, SCT-Fa006, SCT-Fa007, SCT-Fa008, SCT-Fa009, SCT-Fa010, SCT-Fa011, SCT-Fa012, SCT-Fa013, SCT-Fa014, SCT-Fa015, SCT-Fa016, SCT-Fa017, SCT-Fa018, SCT-Fa019, SCT-Fa020, SCT-Fa021, SCT-Fa022, SCT-Fa023, SCT-Fa024, SCT-Fa025, SCT-Fa026, SCT-Fa027, SCT-Fa028, SCT-Fa029, SCT-Fa030, SCT-Fa031, SCT-Fa032, SCT-Fa033, SCT-Fa034, SCT-Fa035, SCT-Fa036, SCT-Fa037, SCT-Fa038, SCT-Fa039, SCT-Fa040, SCT-Fa041, SCT-Fa042, SCT-Fa043, SCT-Fa044, SCT-Fa045, SCT-Fa046, SCT-Fa047, SCT-Fa048, SCT-Fa049, SCT-Fa050, SCT-Fa051, SCT-Fa052, SCT-Fa053, SCT-Fa054, SCT-Fa055, SCT-Fa056, SCT-Fa057, SCT-Fa058, SCT-Fa059, SCT-Fa060, SCT-Fa061, SCT-Fa062, SCT-Fa063, SCT-Fa064, SCT-Fa065, SCT-Fa066, SCT-Fa067, SCT-Fa068, SCT-Fa069, SCT-Fa070, SCT-Fa071, SCT-Fa072, SCT-Fa073, SCT-Fa074, SCT-Fa075, SCT-Fa076, SCT-Fa077, SCT-Fa078, SCT-Fa079, SCT-Fa080, SCT-Fa081, SCT-Fa082, SCT-Fa083, SCT-Fa084, SCT-Fa085, SCT-Fa086, SCT-Fa087, SCT-Fa088, SCT-Fa089, SCT-Fa090, SCT-Fa091, SCT-Fa092, SCT-Fa093, SCT-Fa094, SCT-Fa095, SCT-Fa096, SCT-Fa097, SCT-Fa098, SCT-Fa099, SCT-Fa100, SCT-Fa101, SCT-Fa102, SCT-Fa103, SCT-Fa104, SCT-Fa105, SCT-Fa106, SCT-Fa107, SCT-Fa108, SCT-Fa109, SCT-Fa110, SCT-Fa111, SCT-Fa112, SCT-Fa113, SCT-Fa114, SCT-Fa115, SCT-Fa116, SCT-Fa117, SCT-Fa118, SCT-Fa119, SCT-Fa120, SCT-Fa121, SCT-Fa122, SCT-Fa123, SCT-Fa124, SCT-Fa125, SCT-Fa126, SCT-Fa127, SCT-Fa128, SCT-Fa129, SCT-Fa130, SCT-Fa131, SCT-Fa132, SCT-Fa133, SCT-Fa134, SCT-Fa135, SCT-Fa136, SCT-Fa137, SCT-Fa138, SCT-Fa139, SCT-Fa140, SCT-Fa141, SCT-Fa142, SCT-Fa143, SCT-Fa144, SCT-Fa145, SCT-Fa146, SCT-Fa147, SCT-Fa148, SCT-Fa149, SCT-Fa150, SCT-Fa151, SCT-Fa152, SCT-Fa153, SCT-Fa154, SCT-Fa155, SCT-Fa156, SCT-Fa157, SCT-Fa158, SCT-Fa159, SCT-Fa160, SCT-Fa161, SCT-Fa162, SCT-Fa163, SCT-Fa164, SCT-Fa165, SCT-Fa166, SCT-Fa167, SCT-Fa168, SCT-Fa169, SCT-Fa170, SCT-Fa171, SCT-Fa172, SCT-Fa173, SCT-Fa174, SCT-Fa175, SCT-Fa176, SCT-Fa177, SCT-Fa178, SCT-Fa179, SCT-Fa180, SCT-Fa181, SCT-Fa182, SCT-Fa183, SCT-Fa184, SCT-Fa185, SCT-Fa186, SCT-Fa187, SCT-Fa188, SCT-Fa189, SCT-Fa190, SCT-Fa191, SCT-Fa192, SCT-Fa193, SCT-Fa194, and SCT-Fa195. Each of these is a murine monoclonal antibody.

Additionally, recombinant anti-TIM-3 antibodies, such as chimeric and humanized monoclonal antibodies, comprising both human and non-human portions, which can be made using standard recombinant DNA techniques, are within the scope of the disclosure. Such chimeric and humanized monoclonal antibodies can be produced by recombinant DNA techniques such as, for example, the methods described in U.S. Pat. No. 7,112,421; Better et al. (1988) Science 240:1041-1043; or Liu et al. (1987) Proc. Natl. Acad. Sci. USA 84:3439-3443.

### Antibody Variable Domain Sequence

The antibodies of the disclosure may comprise the heavy chain variable domain sequences of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:14, SEQ ID NO:15, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:90, SEQ ID NO:91, SEQ ID NO:92, SEQ ID NO:93, SEQ ID NO:94, SEQ ID NO:95, SEQ ID NO:96, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO: 144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:156, SEQ ID NO:156, SEQ ID NO:157, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:171, SEQ ID NO:172, SEQ ID NO:173, SEQ ID NO:174, SEQ ID NO:175, SEQ ID NO:175, SEQ ID NO:176, SEQ ID NO:177, SEQ ID NO: 178, SEQ ID NO: 179, SEQ ID NO: 180, SEQ ID NO:181, SEQ ID NO:182, SEQ ID NO:183, SEQ ID NO:184, SEQ ID NO:185, SEQ ID NO:186, or SEQ ID NO:186. The heavy chain variable domain sequences may consist essentially of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:14, SEQ ID NO:15, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:90, SEQ ID NO:91, SEQ ID NO:92, SEQ ID NO:93, SEQ ID NO:94, SEQ ID NO:95, SEQ ID NO:96, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:156, SEQ ID NO:156, SEQ ID NO:157, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:171, SEQ ID NO:172, SEQ ID NO:173, SEQ ID NO:174, SEQ ID NO:175, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, or SEQ ID NO:186.

The antibodies of the disclosure may comprise the light chain variable domain sequences of SEQ ID NO:187, SEQ ID NO:188, SEQ ID NO:189, SEQ ID NO:190, SEQ ID NO:191, SEQ ID NO:191, SEQ ID NO:192, SEQ ID NO:192, SEQ ID NO:192, SEQ ID NO:192, SEQ ID NO:192, SEQ ID NO:193, SEQ ID NO:194, SEQ ID NO:194, SEQ ID NO:195, SEQ ID NO:196, SEQ ID NO:197, SEQ ID NO:198, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:209, SEQ ID NO:209, SEQ ID NO:210, SEQ ID NO:211, SEQ ID NO:211, SEQ ID NO:211, SEQ ID NO:212, SEQ ID NO:213, SEQ ID NO:214, SEQ ID NO:214, SEQ ID NO:214, SEQ ID NO:214, SEQ ID NO:214, SEQ ID NO:214, SEQ ID NO:214, SEQ ID NO:215, SEQ ID NO:216, SEQ ID NO:217, SEQ ID NO:218, SEQ ID NO:219, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:228, SEQ ID NO:229, SEQ ID NO:229, SEQ ID NO:229, SEQ ID NO:229, SEQ ID NO:229, SEQ ID NO:229, SEQ ID NO:229, SEQ ID NO:229, SEQ ID NO:230, SEQ ID NO:230, SEQ ID NO:230, SEQ ID NO:231, SEQ ID NO:231, SEQ ID NO:232, SEQ ID NO:232, SEQ ID NO:233, SEQ ID NO:233, SEQ ID NO:234, SEQ ID NO:235, SEQ ID NO:236, SEQ ID NO:236, SEQ ID NO:236, SEQ ID NO:237, SEQ ID NO:238, SEQ ID NO:239, SEQ ID NO:240, SEQ ID NO: 240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:280, SEQ ID NO:281, SEQ ID NO:281, SEQ ID NO:282, SEQ ID NO:283, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:305, SEQ ID NO:306, SEQ ID NO:307, SEQ ID NO:308, SEQ ID NO:309, SEQ ID NO:310, SEQ ID NO:310, SEQ ID NO:310, SEQ ID NO:310, SEQ ID NO:310, SEQ ID NO:311, SEQ ID NO:312, SEQ ID NO:313, SEQ ID NO:314, SEQ ID NO:314, SEQ ID NO:315, SEQ ID NO:316, SEQ ID NO:317, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:326, SEQ ID NO:327, SEQ ID NO:328, SEQ ID NO:329, SEQ ID NO:330, SEQ ID NO:331, SEQ ID NO:332, or SEQ ID NO:332. The light chain variable domain sequences may consist essentially of SEQ ID NO:187, SEQ ID NO:188, SEQ ID NO:189, SEQ ID NO:190, SEQ ID NO:191, SEQ ID NO:191, SEQ ID NO:192, SEQ ID NO:192, SEQ ID NO:192, SEQ ID NO:192, SEQ ID NO:192, SEQ ID NO:193, SEQ ID NO:194, SEQ ID NO:194, SEQ ID NO:195, SEQ ID NO:196, SEQ ID NO:197, SEQ ID NO:198, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:209, SEQ ID NO:209, SEQ ID NO:210, SEQ ID NO:211, SEQ ID NO:211, SEQ ID NO:211, SEQ ID NO:212, SEQ ID NO:213, SEQ ID NO:214, SEQ ID NO:214, SEQ ID NO:214, SEQ ID NO:214, SEQ ID NO:214, SEQ ID NO:214, SEQ ID NO:214, SEQ ID NO:215, SEQ ID NO:216, SEQ ID NO:217, SEQ ID NO:218, SEQ ID NO:219, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:228, SEQ ID NO:229, SEQ ID NO:229, SEQ ID NO:229, SEQ ID NO:229, SEQ ID NO:229, SEQ ID NO:229, SEQ ID NO:229, SEQ ID NO:229, SEQ ID NO:230, SEQ ID NO:230, SEQ ID NO:230, SEQ ID NO:231, SEQ ID NO:231, SEQ ID NO:232, SEQ ID NO:232, SEQ ID NO:233, SEQ ID NO:233, SEQ ID NO:234, SEQ ID NO:235, SEQ ID NO:236, SEQ ID NO:236, SEQ ID NO:236, SEQ ID NO:237, SEQ ID NO:238, SEQ ID NO:239, SEQ ID NO:240, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:280, SEQ ID NO:281, SEQ ID NO:281, SEQ ID NO:282, SEQ ID NO:283, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:305, SEQ ID NO:306, SEQ ID NO:307, SEQ ID NO:308, SEQ ID NO:309, SEQ ID NO:310, SEQ ID NO:310, SEQ ID NO:310, SEQ ID NO:310, SEQ ID NO:310, SEQ ID NO:311, SEQ ID NO:312, SEQ ID NO:313, SEQ ID NO:314, SEQ ID NO:314, SEQ ID NO:315, SEQ ID NO:316, SEQ ID NO:317, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:326, SEQ ID NO:327, SEQ ID NO:328, SEQ ID NO:329, SEQ ID NO:330, SEQ ID NO:331, SEQ ID NO:332, or SEQ ID NO:332.

The disclosure also provides a variable domain sequence comprising a sequence that is at least 80%, at least 85%, at least 90%, or at least 95% identical to a sequence selected from SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:14, SEQ ID NO:15, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:90, SEQ ID NO:91, SEQ ID NO:92, SEQ ID NO:93, SEQ ID NO:94, SEQ ID NO:95, SEQ ID NO:96, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:156, SEQ ID NO:156, SEQ ID NO:157, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:171, SEQ ID NO:172, SEQ ID NO:173, SEQ ID NO:174, SEQ ID NO:175, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, or SEQ ID NO:186. The disclosure also provides a variable domain sequence comprising a sequence that is at least 80%, at least 85%, at least 90%, or at least 95% identical to a sequence selected from SEQ ID NO:187, SEQ ID NO:188, SEQ ID NO:189, SEQ ID NO:190, SEQ ID NO:191, SEQ ID NO:191, SEQ ID NO:192, SEQ ID NO:192, SEQ ID NO:192, SEQ ID NO:192, SEQ ID NO:192, SEQ ID NO:193, SEQ ID NO:194, SEQ ID NO:194, SEQ ID NO:195, SEQ ID NO:196, SEQ ID NO:197, SEQ ID NO:198, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:209, SEQ ID NO:209, SEQ ID NO:210, SEQ ID NO:211, SEQ ID NO:211, SEQ ID NO:211, SEQ ID NO:212, SEQ ID NO:213, SEQ ID NO:214, SEQ ID NO:214, SEQ ID NO:214, SEQ ID NO:214, SEQ ID NO:214, SEQ ID NO:214, SEQ ID NO:214, SEQ ID NO:215, SEQ ID NO:216, SEQ ID NO:217, SEQ ID NO:218, SEQ ID NO:219, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:228, SEQ ID NO:229, SEQ ID NO:229, SEQ ID NO:229, SEQ ID NO:229, SEQ ID NO:229, SEQ ID NO:229, SEQ ID NO:229, SEQ ID NO:229, SEQ ID NO:230, SEQ ID NO:230, SEQ ID NO:230, SEQ ID NO:231, SEQ ID NO:231, SEQ ID NO:232, SEQ ID NO:232, SEQ ID NO:233, SEQ ID NO:233, SEQ ID NO:234, SEQ ID NO:235, SEQ ID NO:236, SEQ ID NO:236, SEQ ID NO:236, SEQ ID NO:237, SEQ ID NO:238, SEQ ID NO:239, SEQ ID NO:240, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:280, SEQ ID NO:281, SEQ ID NO:281, SEQ ID NO:282, SEQ ID NO:283, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:305, SEQ ID NO:306, SEQ ID NO:307, SEQ ID NO:308, SEQ ID NO:309, SEQ ID NO:310, SEQ ID NO:310, SEQ ID NO:310, SEQ ID NO:310, SEQ ID NO:310, SEQ ID NO:311, SEQ ID NO:312, SEQ ID NO:313, SEQ ID NO:314, SEQ ID NO:314, SEQ ID NO:315, SEQ ID NO:316, SEQ ID NO:317, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:326, SEQ ID NO:327, SEQ ID NO:328, SEQ ID NO:329, SEQ ID NO:330, SEQ ID NO:331, SEQ ID NO:332, or SEQ ID NO:332. The disclosure also provides antibodies comprising a heavy chain variable domain sequence that is at least 80%, at least 85%, at least 90%, or at least 95% identical to SEQ ID NO:4 and a light chain variable domain sequence that is at least 80%, at least 85%, at least 90%, or at least 95% identical to SEQ ID NO:317. The disclosure also provides antibodies comprising a heavy chain variable domain sequence that is at least 80%, at least 85%, at least 90%, or at least 95% identical to SEQ ID NO:5 and a light chain variable domain sequence that is at least 80%, at least 85%, at least 90%, or at least 95% identical to SEQ ID NO:211. The disclosure also provides antibodies comprising a heavy chain variable domain sequence that is at least 80%, at least 85%, at least 90%, or at least 95% identical to SEQ ID NO:6 and a light chain variable domain sequence that is at least 80%, at least 85%, at least 90%, or at least 95% identical to SEQ ID NO:302. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:223. The disclosure also provides antibodies comprising a heavy chain variable domain sequence that is at least 80%, at least 85%, at least 90%, or at least 95% identical to SEQ ID NO:8 and a light chain variable domain sequence that is at least 80%, at least 85%, at least 90%, or at least 95% identical to SEQ ID NO:260. The disclosure also provides antibodies comprising a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:325. The disclosure also provides antibodies comprising a heavy chain variable domain sequence that is at least 80%, at least 85%, at least 90%, or at least 95% identical to SEQ ID NO:10 and a light chain variable domain sequence that is at least 80%, at least 85%, at least 90%, or at least 95% identical to SEQ ID NO:305. The disclosure also provides antibodies comprising a heavy chain variable domain sequence that is at least 80%, at least 85%, at least 90%, or at least 95% identical to SEQ ID NO:11 and a light chain variable domain sequence that is at least 80%, at least 85%, at least 90%, or at least 95% identical to SEQ ID NO:319. The disclosure also provides antibodies comprising a heavy chain variable domain sequence that is at least 80%, at least 85%, at least 90%, or at least 95% identical to SEQ ID NO:12 and a light chain variable domain sequence that is at least 80%, at least 85%, at least 90%, or at least 95% identical to SEQ ID NO:241. The disclosure also provides antibodies comprising a heavy chain variable domain sequence that is at least 80%, at least 85%, at least 90%, or at least 95% identical to SEQ ID NO:13 and a light chain variable domain sequence that is at least 80%, at least 85%, at least 90%, or at least 95% identical to SEQ ID NO:306. The disclosure also provides antibodies comprising a heavy chain variable domain sequence that is at least 80%, at least 85%, at least 90%, or at least 95% identical to SEQ ID NO:14 and a light chain variable domain sequence that is at least 80%, at least 85%, at least 90%, or at least 95% identical to SEQ ID NO:315. The disclosure also provides antibodies comprising a heavy chain variable domain sequence that is at least 80%, at least 85%, at least 90%, or at least 95% identical to SEQ ID NO:14 and a light chain variable domain sequence that is at least 80%, at least 85%, at least 90%, or at least 95% identical to SEQ ID NO:316. The disclosure also provides antibodies comprising a heavy chain variable domain sequence that is at least 80%, at least 85%, at least 90%, or at least 95% identical to SEQ ID NO:15 and a light chain variable domain sequence that is at least 80%, at least 85%, at least 90%, or at least 95% identical to SEQ ID NO:312. The disclosure also provides antibodies comprising a heavy chain variable domain sequence that is at least 80%, at least 85%, at least 90%, or at least 95% identical to SEQ ID NO:15 and a light chain variable domain sequence that is at least 80%, at least 85%, at least 90%, or at least 95% identical to SEQ ID NO:314. The disclosure also provides antibodies comprising a heavy chain variable domain sequence that is at least 80%, at least 85%, at least 90%, or at least 95% identical to SEQ ID NO:16 and a light chain variable domain sequence that is at least 80%, at least 85%, at least 90%, or at least 95% identical to SEQ ID NO:314. The disclosure also provides antibodies comprising a heavy chain variable domain sequence that is at least 80%, at least 85%, at least 90%, or at least 95% identical to SEQ ID NO:17 and a light chain variable domain sequence that is at least 80%, at least 85%, at least 90%, or at least 95% identical to SEQ ID NO:288. The disclosure also provides antibodies comprising a heavy chain variable domain sequence that is at least 80%, at least 85%, at least 90%, or at least 95% identical to SEQ ID NO:18 and a light chain variable domain sequence that is at least 80%, at least 85%, at least 90%, or at least 95% identical to SEQ ID NO:313. The disclosure also provides antibodies comprising a heavy chain variable domain sequence that is at least 80%, at least 85%, at least 90%, or at least 95% identical to SEQ ID NO:19 and a light chain variable domain sequence that is at least 80%, at least 85%, at least 90%, or at least 95% identical to SEQ ID NO:298. The disclosure also provides antibodies comprising a heavy chain variable domain sequence that is at least 80%, at least 85%, at least 90%, or at least 95% identical to SEQ ID NO:20 and a light chain variable domain sequence that is at least 80%, at least 85%, at least 90%, or at least 95% identical to SEQ ID NO:307. The disclosure also provides antibodies comprising a heavy chain variable domain sequence that is at least 80%, at least 85%, at least 90%, or at least 95% identical to SEQ ID NO:21 and a light chain variable domain sequence that is at least 80%, at least 85%, at least 90%, or at least 95% identical to SEQ ID NO:246. The disclosure also provides antibodies comprising a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:261. The disclosure also provides antibodies comprising a heavy chain variable domain sequence that is at least 80%, at least 85%, at least 90%, or at least 95% identical to SEQ ID NO:23 and a light chain variable domain sequence that is at least 80%, at least 85%, at least 90%, or at least 95% identical to SEQ ID NO:330. The disclosure also provides antibodies comprising a heavy chain variable domain sequence that is at least 80%, at least 85%, at least 90%, or at least 95% identical to SEQ ID NO:24 and a light chain variable domain sequence that is at least 80%, at least 85%, at least 90%, or at least 95% identical to SEQ ID NO:237. The disclosure also provides antibodies comprising a heavy chain variable domain sequence that is at least 80%, at least 85%, at least 90%, or at least 95% identical to SEQ ID NO:25 and a light chain variable domain sequence that is at least 80%, at least 85%, at least 90%, or at least 95% identical to SEQ ID NO:238. The disclosure also provides antibodies comprising a heavy chain variable domain sequence that is at least 80%, at least 85%, at least 90%, or at least 95% identical to SEQ ID NO:26 and a light chain variable domain sequence that is at least 80%, at least 85%, at least 90%, or at least 95% identical to SEQ ID NO:236. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:236. The disclosure also provides antibodies comprising a heavy chain variable domain sequence that is at least 80%, at least 85%, at least 90%, or at least 95% identical to SEQ ID NO:28 and a light chain variable domain sequence that is at least 80%, at least 85%, at least 90%, or at least 95% identical to SEQ ID NO:231. The disclosure also provides antibodies comprising a heavy chain variable domain sequence that is at least 80%, at least 85%, at least 90%, or at least 95% identical to SEQ ID NO:29 and a light chain variable domain sequence that is at least 80%, at least 85%, at least 90%, or at least 95% identical to SEQ ID NO:232. The disclosure also provides antibodies comprising a heavy chain variable domain sequence that is at least 80%, at least 85%, at least 90%, or at least 95% identical to SEQ ID NO:30 and a light chain variable domain sequence that is at least 80%, at least 85%, at least 90%, or at least 95% identical to SEQ ID NO:224. The disclosure also provides antibodies comprising a heavy chain variable domain sequence that is at least 80%, at least 85%, at least 90%, or at least 95% identical to SEQ ID NO:31 and a light chain variable domain sequence that is at least 80%, at least 85%, at least 90%, or at least 95% identical to SEQ ID NO:235. The disclosure also provides antibodies comprising a heavy chain variable domain sequence that is at least 80%, at least 85%, at least 90%, or at least 95% identical to SEQ ID NO:32 and a light chain variable domain sequence that is at least 80%, at least 85%, at least 90%, or at least 95% identical to SEQ ID NO:240. The disclosure also provides antibodies comprising a heavy chain variable domain sequence that is at least 80%, at least 85%, at least 90%, or at least 95% identical to SEQ ID NO:33 and a light chain variable domain sequence that is at least 80%, at least 85%, at least 90%, or at least 95% identical to SEQ ID NO:220. The disclosure also provides antibodies comprising a heavy chain variable domain sequence that is at least 80%, at least 85%, at least 90%, or at least 95% identical to SEQ ID NO:34 and a light chain variable domain sequence that is at least 80%, at least 85%, at least 90%, or at least 95% identical to SEQ ID NO:221. The disclosure also provides antibodies comprising a heavy chain variable domain sequence that is at least 80%, at least 85%, at least 90%, or at least 95% identical to SEQ ID NO:35 and a light chain variable domain sequence that is at least 80%, at least 85%, at least 90%, or at least 95% identical to SEQ ID NO:240. The disclosure also provides antibodies comprising a heavy chain variable domain sequence that is at least 80%, at least 85%, at least 90%, or at least 95% identical to SEQ ID NO:35 and a light chain variable domain sequence that is at least 80%, at least 85%, at least 90%, or at least 95% identical to SEQ ID NO:317. The disclosure also provides antibodies comprising a heavy chain variable domain sequence that is at least 80%, at least 85%, at least 90%, or at least 95% identical to SEQ ID NO:36 and a light chain variable domain sequence that is at least 80%, at least 85%, at least 90%, or at least 95% identical to SEQ ID NO:277. The disclosure also provides antibodies comprising a heavy chain variable domain sequence that is at least 80%, at least 85%, at least 90%, or at least 95% identical to SEQ ID NO:37 and a light chain variable domain sequence that is at least 80%, at least 85%, at least 90%, or at least 95% identical to SEQ ID NO:239. The disclosure also provides antibodies comprising a heavy chain variable domain sequence that is at least 80%, at least 85%, at least 90%, or at least 95% identical to SEQ ID NO:38 and a light chain variable domain sequence that is at least 80%, at least 85%, at least 90%, or at least 95% identical to SEQ ID NO:236. The disclosure also provides antibodies comprising a heavy chain variable domain sequence that is at least 80%, at least 85%, at least 90%, or at least 95% identical to SEQ ID NO:39 and a light chain variable domain sequence that is at least 80%, at least 85%, at least 90%, or at least 95% identical to SEQ ID NO:228. The disclosure also provides antibodies comprising a heavy chain variable domain sequence that is at least 80%, at least 85%, at least 90%, or at least 95% identical to SEQ ID NO:40 and a light chain variable domain sequence that is at least 80%, at least 85%, at least 90%, or at least 95% identical to SEQ ID NO:228. The disclosure also provides antibodies comprising a heavy chain variable domain sequence that is at least 80%, at least 85%, at least 90%, or at least 95% identical to SEQ ID NO:41 and a light chain variable domain sequence that is at least 80%, at least 85%, at least 90%, or at least 95% identical to SEQ ID NO:231. The disclosure also provides antibodies comprising a heavy chain variable domain sequence that is at least 80%, at least 85%, at least 90%, or at least 95% identical to SEQ ID NO:42 and a light chain variable domain sequence that is at least 80%, at least 85%, at least 90%, or at least 95% identical to SEQ ID NO:232. The disclosure also provides antibodies comprising a heavy chain variable domain sequence that is at least 80%, at least 85%, at least 90%, or at least 95% identical to SEQ ID NO:43 and a light chain variable domain sequence that is at least 80%, at least 85%, at least 90%, or at least 95% identical to SEQ ID NO:233. The disclosure also provides antibodies comprising a heavy chain variable domain sequence that is at least 80%, at least 85%, at least 90%, or at least 95% identical to SEQ ID NO:44 and a light chain variable domain sequence that is at least 80%, at least 85%, at least 90%, or at least 95% identical to SEQ ID NO:233. The disclosure also provides antibodies comprising a heavy chain variable domain sequence that is at least 80%, at least 85%, at least 90%, or at least 95% identical to SEQ ID NO:45 and a light chain variable domain sequence that is at least 80%, at least 85%, at least 90%, or at least 95% identical to SEQ ID NO:269. The disclosure also provides antibodies comprising a heavy chain variable domain sequence that is at least 80%, at least 85%, at least 90%, or at least 95% identical to SEQ ID NO:46 and a light chain variable domain sequence that is at least 80%, at least 85%, at least 90%, or at least 95% identical to SEQ ID NO:305. The disclosure also provides antibodies comprising a heavy chain variable domain sequence that is at least 80%, at least 85%, at least 90%, or at least 95% identical to SEQ ID NO:47 and a light chain variable domain sequence that is at least 80%, at least 85%, at least 90%, or at least 95% identical to SEQ ID NO:284. The disclosure also provides antibodies comprising a heavy chain variable domain sequence that is at least 80%, at least 85%, at least 90%, or at least 95% identical to SEQ ID NO:48 and a light chain variable domain sequence that is at least 80%, at least 85%, at least 90%, or at least 95% identical to SEQ ID NO:283. The disclosure also provides antibodies comprising a heavy chain variable domain sequence that is at least 80%, at least 85%, at least 90%, or at least 95% identical to SEQ ID NO:49 and a light chain variable domain sequence that is at least 80%, at least 85%, at least 90%, or at least 95% identical to SEQ ID NO:283. The disclosure also provides antibodies comprising a heavy chain variable domain sequence that is at least 80%, at least 85%, at least 90%, or at least 95% identical to SEQ ID NO:50 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:51 and a light chain variable domain sequence that is at least 80%, at least 85%, at least 90%, or at least 95% identical to SEQ ID NO:262. The disclosure also provides antibodies comprising a heavy chain variable domain sequence that is at least 80%, at least 85%, at least 90%, or at least 95% identical to SEQ ID NO:52 and a light chain variable domain sequence that is at least 80%, at least 85%, at least 90%, or at least 95% identical to SEQ ID NO:206. The disclosure also provides antibodies comprising a heavy chain variable domain sequence that is at least 80%, at least 85%, at least 90%, or at least 95% identical to SEQ ID NO:53 and a light chain variable domain sequence that is at least 80%, at least 85%, at least 90%, or at least 95% identical to SEQ ID NO:205. The disclosure also provides antibodies comprising a heavy chain variable domain sequence that is at least 80%, at least 85%, at least 90%, or at least 95% identical to SEQ ID NO:54 and a light chain variable domain sequence that is at least 80%, at least 85%, at least 90%, or at least 95% identical to SEQ ID NO:204. The disclosure also provides antibodies comprising a heavy chain variable domain sequence that is at least 80%, at least 85%, at least 90%, or at least 95% identical to SEQ ID NO:55 and a light chain variable domain sequence that is at least 80%, at least 85%, at least 90%, or at least 95% identical to SEQ ID NO:202. The disclosure also provides antibodies comprising a heavy chain variable domain sequence that is at least 80%, at least 85%, at least 90%, or at least 95% identical to SEQ ID NO:56 and a light chain variable domain sequence that is at least 80%, at least 85%, at least 90%, or at least 95% identical to SEQ ID NO:263. The disclosure also provides antibodies comprising a heavy chain variable domain sequence that is at least 80%, at least 85%, at least 90%, or at least 95% identical to SEQ ID NO:57 and a light chain variable domain sequence that is at least 80%, at least 85%, at least 90%, or at least 95% identical to SEQ ID NO:310. The disclosure also provides antibodies comprising a heavy chain variable domain sequence that is at least 80%, at least 85%, at least 90%, or at least 95% identical to SEQ ID NO:58 and a light chain variable domain sequence that is at least 80%, at least 85%, at least 90%, or at least 95% identical to SEQ ID NO:320. The disclosure also provides antibodies comprising a heavy chain variable domain sequence that is at least 80%, at least 85%, at least 90%, or at least 95% identical to SEQ ID NO:59 and a light chain variable domain sequence that is at least 80%, at least 85%, at least 90%, or at least 95% identical to SEQ ID NO:259. The disclosure also provides antibodies comprising a heavy chain variable domain sequence that is at least 80%, at least 85%, at least 90%, or at least 95% identical to SEQ ID NO:60 and a light chain variable domain sequence that is at least 80%, at least 85%, at least 90%, or at least 95% identical to SEQ ID NO:287. The disclosure also provides antibodies comprising a heavy chain variable domain sequence that is at least 80%, at least 85%, at least 90%, or at least 95% identical to SEQ ID NO:61 and a light chain variable domain sequence that is at least 80%, at least 85%, at least 90%, or at least 95% identical to SEQ ID NO:203. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:310. The disclosure also provides antibodies comprising a heavy chain variable domain sequence that is at least 80%, at least 85%, at least 90%, or at least 95% identical to SEQ ID NO:63 and a light chain variable domain sequence that is at least 80%, at least 85%, at least 90%, or at least 95% identical to SEQ ID NO:311. The disclosure also provides antibodies comprising a heavy chain variable domain sequence that is at least 80%, at least 85%, at least 90%, or at least 95% identical to SEQ ID NO:64 and a light chain variable domain sequence that is at least 80%, at least 85%, at least 90%, or at least 95% identical to SEQ ID NO:265. The disclosure also provides antibodies comprising a heavy chain variable domain sequence that is at least 80%, at least 85%, at least 90%, or at least 95% identical to SEQ ID NO:65 and a light chain variable domain sequence that is at least 80%, at least 85%, at least 90%, or at least 95% identical to SEQ ID NO:310. The disclosure also provides antibodies comprising a heavy chain variable domain sequence that is at least 80%, at least 85%, at least 90%, or at least 95% identical to SEQ ID NO:66 and a light chain variable domain sequence that is at least 80%, at least 85%, at least 90%, or at least 95% identical to SEQ ID NO:308. The disclosure also provides antibodies comprising a heavy chain variable domain sequence that is at least 80%, at least 85%, at least 90%, or at least 95% identical to SEQ ID NO:67 and a light chain variable domain sequence that is at least 80%, at least 85%, at least 90%, or at least 95% identical to SEQ ID NO:290. The disclosure also provides antibodies comprising a heavy chain variable domain sequence that is at least 80%, at least 85%, at least 90%, or at least 95% identical to SEQ ID NO:67 and a light chain variable domain sequence that is at least 80%, at least 85%, at least 90%, or at least 95% identical to SEQ ID NO:310. The disclosure also provides antibodies comprising a heavy chain variable domain sequence that is at least 80%, at least 85%, at least 90%, or at least 95% identical to SEQ ID NO:68 and a light chain variable domain sequence that is at least 80%, at least 85%, at least 90%, or at least 95% identical to SEQ ID NO:309. The disclosure also provides antibodies comprising a heavy chain variable domain sequence that is at least 80%, at least 85%, at least 90%, or at least 95% identical to SEQ ID NO:69 and a light chain variable domain sequence that is at least 80%, at least 85%, at least 90%, or at least 95% identical to SEQ ID NO:310. The disclosure also provides antibodies comprising a heavy chain variable domain sequence that is at least 80%, at least 85%, at least 90%, or at least 95% identical to SEQ ID NO:70 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:71 and a light chain variable domain sequence that is at least 80%, at least 85%, at least 90%, or at least 95% identical to SEQ ID NO:300. The disclosure also provides antibodies comprising a heavy chain variable domain sequence that is at least 80%, at least 85%, at least 90%, or at least 95% identical to SEQ ID NO:72 and a light chain variable domain sequence that is at least 80%, at least 85%, at least 90%, or at least 95% identical to SEQ ID NO:324. The disclosure also provides antibodies comprising a heavy chain variable domain sequence that is at least 80%, at least 85%, at least 90%, or at least 95% identical to SEQ ID NO:73 and a light chain variable domain sequence that is at least 80%, at least 85%, at least 90%, or at least 95% identical to SEQ ID NO:326. The disclosure also provides antibodies comprising a heavy chain variable domain sequence that is at least 80%, at least 85%, at least 90%, or at least 95% identical to SEQ ID NO:74 and a light chain variable domain sequence that is at least 80%, at least 85%, at least 90%, or at least 95% identical to SEQ ID NO:326. The disclosure also provides antibodies comprising a heavy chain variable domain sequence that is at least 80%, at least 85%, at least 90%, or at least 95% identical to SEQ ID NO:75 and a light chain variable domain sequence that is at least 80%, at least 85%, at least 90%, or at least 95% identical to SEQ ID NO:243. The disclosure also provides antibodies comprising a heavy chain variable domain sequence that is at least 80%, at least 85%, at least 90%, or at least 95% identical to SEQ ID NO:76 and a light chain variable domain sequence that is at least 80%, at least 85%, at least 90%, or at least 95% identical to SEQ ID NO:217. The disclosure also provides antibodies comprising a heavy chain variable domain sequence that is at least 80%, at least 85%, at least 90%, or at least 95% identical to SEQ ID NO:77 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO:78 and a light chain variable domain sequence that is at least 80%, at least 85%, at least 90%, or at least 95% identical to SEQ ID NO:211. The disclosure also provides antibodies comprising a heavy chain variable domain sequence that is at least 80%, at least 85%, at least 90%, or at least 95% identical to SEQ ID NO:79 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO:80 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO:81 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO:82 and a light chain variable domain sequence that is at least 80%, at least 85%, at least 90%, or at least 95% identical to SEQ ID NO:213. The disclosure also provides antibodies comprising a heavy chain variable domain sequence that is at least 80%, at least 85%, at least 90%, or at least 95% identical to SEQ ID NO:83 and a light chain variable domain sequence that is at least 80%, at least 85%, at least 90%, or at least 95% identical to SEQ ID NO:215. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:332. The disclosure also provides antibodies comprising a heavy chain variable domain sequence that is at least 80%, at least 85%, at least 90%, or at least 95% identical to SEQ ID NO:85 and a light chain variable domain sequence that is at least 80%, at least 85%, at least 90%, or at least 95% identical to SEQ ID NO:328. The disclosure also provides antibodies comprising a heavy chain variable domain sequence that is at least 80%, at least 85%, at least 90%, or at least 95% identical to SEQ ID NO:86 and a light chain variable domain sequence that is at least 80%, at least 85%, at least 90%, or at least 95% identical to SEQ ID NO:331. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:329. The disclosure also provides antibodies comprising a heavy chain variable domain sequence that is at least 80%, at least 85%, at least 90%, or at least 95% identical to SEQ ID NO:88 and a light chain variable domain sequence that is at least 80%, at least 85%, at least 90%, or at least 95% identical to SEQ ID NO:332. The disclosure also provides antibodies comprising a heavy chain variable domain sequence that is at least 80%, at least 85%, at least 90%, or at least 95% identical to SEQ ID NO:89 and a light chain variable domain sequence that is at least 80%, at least 85%, at least 90%, or at least 95% identical to SEQ ID NO:198. The disclosure also provides antibodies comprising a heavy chain variable domain sequence that is at least 80%, at least 85%, at least 90%, or at least 95% identical to SEQ ID NO:90 and a light chain variable domain sequence that is at least 80%, at least 85%, at least 90%, or at least 95% identical to SEQ ID NO: 197. The disclosure also provides antibodies comprising a heavy chain variable domain sequence that is at least 80%, at least 85%, at least 90%, or at least 95% identical to SEQ ID NO:90 and a light chain variable domain sequence that is at least 80%, at least 85%, at least 90%, or at least 95% identical to SEQ ID NO:198. The disclosure also provides antibodies comprising a heavy chain variable domain sequence that is at least 80%, at least 85%, at least 90%, or at least 95% identical to SEQ ID NO:91 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO:92 and a light chain variable domain sequence that is at least 80%, at least 85%, at least 90%, or at least 95% identical to SEQ ID NO:299. The disclosure also provides antibodies comprising a heavy chain variable domain sequence that is at least 80%, at least 85%, at least 90%, or at least 95% identical to SEQ ID NO:93 and a light chain variable domain sequence that is at least 80%, at least 85%, at least 90%, or at least 95% identical to SEQ ID NO:285. The disclosure also provides antibodies comprising a heavy chain variable domain sequence that is at least 80%, at least 85%, at least 90%, or at least 95% identical to SEQ ID NO:94 and a light chain variable domain sequence that is at least 80%, at least 85%, at least 90%, or at least 95% identical to SEQ ID NO:267. The disclosure also provides antibodies comprising a heavy chain variable domain sequence that is at least 80%, at least 85%, at least 90%, or at least 95% identical to SEQ ID NO:95 and a light chain variable domain sequence that is at least 80%, at least 85%, at least 90%, or at least 95% identical to SEQ ID NO:222. The disclosure also provides antibodies comprising a heavy chain variable domain sequence that is at least 80%, at least 85%, at least 90%, or at least 95% identical to SEQ ID NO:96 and a light chain variable domain sequence that is at least 80%, at least 85%, at least 90%, or at least 95% identical to SEQ ID NO:227. The disclosure also provides antibodies comprising a heavy chain variable domain sequence that is at least 80%, at least 85%, at least 90%, or at least 95% identical to SEQ ID NO:96 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO:97 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO:98 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO:100 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO: 101 and a light chain variable domain sequence that is at least 80%, at least 85%, at least 90%, or at least 95% identical to SEQ ID NO:230. The disclosure also provides antibodies comprising a heavy chain variable domain sequence that is at least 80%, at least 85%, at least 90%, or at least 95% identical to SEQ ID NO:102 and a light chain variable domain sequence that is at least 80%, at least 85%, at least 90%, or at least 95% identical to SEQ ID NO:225. The disclosure also provides antibodies comprising a heavy chain variable domain sequence that is at least 80%, at least 85%, at least 90%, or at least 95% identical to SEQ ID NO: 103 and a light chain variable domain sequence that is at least 80%, at least 85%, at least 90%, or at least 95% identical to SEQ ID NO:234. The disclosure also provides antibodies comprising a heavy chain variable domain sequence that is at least 80%, at least 85%, at least 90%, or at least 95% identical to SEQ ID NO:104 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO: 105 and a light chain variable domain sequence that is at least 80%, at least 85%, at least 90%, or at least 95% identical to SEQ ID NO:230. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:230. The disclosure also provides antibodies comprising a heavy chain variable domain sequence that is at least 80%, at least 85%, at least 90%, or at least 95% identical to SEQ ID NO:107 and a light chain variable domain sequence that is at least 80%, at least 85%, at least 90%, or at least 95% identical to SEQ ID NO:229. The disclosure also provides antibodies comprising a heavy chain variable domain sequence that is at least 80%, at least 85%, at least 90%, or at least 95% identical to SEQ ID NO:108 and a light chain variable domain sequence that is at least 80%, at least 85%, at least 90%, or at least 95% identical to SEQ ID NO:282. The disclosure also provides antibodies comprising a heavy chain variable domain sequence that is at least 80%, at least 85%, at least 90%, or at least 95% identical to SEQ ID NO:109 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO:110 and a light chain variable domain sequence that is at least 80%, at least 85%, at least 90%, or at least 95% identical to SEQ ID NO:212. The disclosure also provides antibodies comprising a heavy chain variable domain sequence that is at least 80%, at least 85%, at least 90%, or at least 95% identical to SEQ ID NO:111 and a light chain variable domain sequence that is at least 80%, at least 85%, at least 90%, or at least 95% identical to SEQ ID NO:268. The disclosure also provides antibodies comprising a heavy chain variable domain sequence that is at least 80%, at least 85%, at least 90%, or at least 95% identical to SEQ ID NO:112 and a light chain variable domain sequence that is at least 80%, at least 85%, at least 90%, or at least 95% identical to SEQ ID NO:266. The disclosure also provides antibodies comprising a heavy chain variable domain sequence that is at least 80%, at least 85%, at least 90%, or at least 95% identical to SEQ ID NO:113 and a light chain variable domain sequence that is at least 80%, at least 85%, at least 90%, or at least 95% identical to SEQ ID NO:280. The disclosure also provides antibodies comprising a heavy chain variable domain sequence that is at least 80%, at least 85%, at least 90%, or at least 95% identical to SEQ ID NO:114 and a light chain variable domain sequence that is at least 80%, at least 85%, at least 90%, or at least 95% identical to SEQ ID NO:266. The disclosure also provides antibodies comprising a heavy chain variable domain sequence that is at least 80%, at least 85%, at least 90%, or at least 95% identical to SEQ ID NO:115 and a light chain variable domain sequence that is at least 80%, at least 85%, at least 90%, or at least 95% identical to SEQ ID NO:187. The disclosure also provides antibodies comprising a heavy chain variable domain sequence that is at least 80%, at least 85%, at least 90%, or at least 95% identical to SEQ ID NO:116 and a light chain variable domain sequence that is at least 80%, at least 85%, at least 90%, or at least 95% identical to SEQ ID NO:280. The disclosure also provides antibodies comprising a heavy chain variable domain sequence that is at least 80%, at least 85%, at least 90%, or at least 95% identical to SEQ ID NO:117 and a light chain variable domain sequence that is at least 80%, at least 85%, at least 90%, or at least 95% identical to SEQ ID NO:281. The disclosure also provides antibodies comprising a heavy chain variable domain sequence that is at least 80%, at least 85%, at least 90%, or at least 95% identical to SEQ ID NO:118 and a light chain variable domain sequence that is at least 80%, at least 85%, at least 90%, or at least 95% identical to SEQ ID NO:278. The disclosure also provides antibodies comprising a heavy chain variable domain sequence that is at least 80%, at least 85%, at least 90%, or at least 95% identical to SEQ ID NO:119 and a light chain variable domain sequence that is at least 80%, at least 85%, at least 90%, or at least 95% identical to SEQ ID NO:250. The disclosure also provides antibodies comprising a heavy chain variable domain sequence that is at least 80%, at least 85%, at least 90%, or at least 95% identical to SEQ ID NO:120 and a light chain variable domain sequence that is at least 80%, at least 85%, at least 90%, or at least 95% identical to SEQ ID NO:253. The disclosure also provides antibodies comprising a heavy chain variable domain sequence that is at least 80%, at least 85%, at least 90%, or at least 95% identical to SEQ ID NO:121 and a light chain variable domain sequence that is at least 80%, at least 85%, at least 90%, or at least 95% identical to SEQ ID NO:249. The disclosure also provides antibodies comprising a heavy chain variable domain sequence that is at least 80%, at least 85%, at least 90%, or at least 95% identical to SEQ ID NO:122 and a light chain variable domain sequence that is at least 80%, at least 85%, at least 90%, or at least 95% identical to SEQ ID NO:256. The disclosure also provides antibodies comprising a heavy chain variable domain sequence that is at least 80%, at least 85%, at least 90%, or at least 95% identical to SEQ ID NO:123 and a light chain variable domain sequence that is at least 80%, at least 85%, at least 90%, or at least 95% identical to SEQ ID NO:252. The disclosure also provides antibodies comprising a heavy chain variable domain sequence that is at least 80%, at least 85%, at least 90%, or at least 95% identical to SEQ ID NO:124 and a light chain variable domain sequence that is at least 80%, at least 85%, at least 90%, or at least 95% identical to SEQ ID NO:257. The disclosure also provides antibodies comprising a heavy chain variable domain sequence that is at least 80%, at least 85%, at least 90%, or at least 95% identical to SEQ ID NO:125 and a light chain variable domain sequence that is at least 80%, at least 85%, at least 90%, or at least 95% identical to SEQ ID NO:219. The disclosure also provides antibodies comprising a heavy chain variable domain sequence that is at least 80%, at least 85%, at least 90%, or at least 95% identical to SEQ ID NO:126 and a light chain variable domain sequence that is at least 80%, at least 85%, at least 90%, or at least 95% identical to SEQ ID NO:219. The disclosure also provides antibodies comprising a heavy chain variable domain sequence that is at least 80%, at least 85%, at least 90%, or at least 95% identical to SEQ ID NO:127 and a light chain variable domain sequence that is at least 80%, at least 85%, at least 90%, or at least 95% identical to SEQ ID NO:251. The disclosure also provides antibodies comprising a heavy chain variable domain sequence that is at least 80%, at least 85%, at least 90%, or at least 95% identical to SEQ ID NO:128 and a light chain variable domain sequence that is at least 80%, at least 85%, at least 90%, or at least 95% identical to SEQ ID NO:245. The disclosure also provides antibodies comprising a heavy chain variable domain sequence that is at least 80%, at least 85%, at least 90%, or at least 95% identical to SEQ ID NO:129 and a light chain variable domain sequence that is at least 80%, at least 85%, at least 90%, or at least 95% identical to SEQ ID NO:254. The disclosure also provides antibodies comprising a heavy chain variable domain sequence that is at least 80%, at least 85%, at least 90%, or at least 95% identical to SEQ ID NO:130 and a light chain variable domain sequence that is at least 80%, at least 85%, at least 90%, or at least 95% identical to SEQ ID NO:244. The disclosure also provides antibodies comprising a heavy chain variable domain sequence that is at least 80%, at least 85%, at least 90%, or at least 95% identical to SEQ ID NO:131 and a light chain variable domain sequence that is at least 80%, at least 85%, at least 90%, or at least 95% identical to SEQ ID NO:272. The disclosure also provides antibodies comprising a heavy chain variable domain sequence that is at least 80%, at least 85%, at least 90%, or at least 95% identical to SEQ ID NO:132 and a light chain variable domain sequence that is at least 80%, at least 85%, at least 90%, or at least 95% identical to SEQ ID NO:271. The disclosure also provides antibodies comprising a heavy chain variable domain sequence that is at least 80%, at least 85%, at least 90%, or at least 95% identical to SEQ ID NO:133 and a light chain variable domain sequence that is at least 80%, at least 85%, at least 90%, or at least 95% identical to SEQ ID NO:289. The disclosure also provides antibodies comprising a heavy chain variable domain sequence that is at least 80%, at least 85%, at least 90%, or at least 95% identical to SEQ ID NO:134 and a light chain variable domain sequence that is at least 80%, at least 85%, at least 90%, or at least 95% identical to SEQ ID NO:301. The disclosure also provides antibodies comprising a heavy chain variable domain sequence that is at least 80%, at least 85%, at least 90%, or at least 95% identical to SEQ ID NO: 135 and a light chain variable domain sequence that is at least 80%, at least 85%, at least 90%, or at least 95% identical to SEQ ID NO:292. The disclosure also provides antibodies comprising a heavy chain variable domain sequence that is at least 80%, at least 85%, at least 90%, or at least 95% identical to SEQ ID NO:136 and a light chain variable domain sequence that is at least 80%, at least 85%, at least 90%, or at least 95% identical to SEQ ID NO: 195. The disclosure also provides antibodies comprising a heavy chain variable domain sequence that is at least 80%, at least 85%, at least 90%, or at least 95% identical to SEQ ID NO: 137 and a light chain variable domain sequence that is at least 80%, at least 85%, at least 90%, or at least 95% identical to SEQ ID NO:291. The disclosure also provides antibodies comprising a heavy chain variable domain sequence that is at least 80%, at least 85%, at least 90%, or at least 95% identical to SEQ ID NO:138 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO: 139 and a light chain variable domain sequence that is at least 80%, at least 85%, at least 90%, or at least 95% identical to SEQ ID NO:296. The disclosure also provides antibodies comprising a heavy chain variable domain sequence that is at least 80%, at least 85%, at least 90%, or at least 95% identical to SEQ ID NO:140 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO: 141 and a light chain variable domain sequence that is at least 80%, at least 85%, at least 90%, or at least 95% identical to SEQ ID NO:255. The disclosure also provides antibodies comprising a heavy chain variable domain sequence that is at least 80%, at least 85%, at least 90%, or at least 95% identical to SEQ ID NO:142 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO: 143 and a light chain variable domain sequence that is at least 80%, at least 85%, at least 90%, or at least 95% identical to SEQ ID NO:211. The disclosure also provides antibodies comprising a heavy chain variable domain sequence that is at least 80%, at least 85%, at least 90%, or at least 95% identical to SEQ ID NO:144 and a light chain variable domain sequence that is at least 80%, at least 85%, at least 90%, or at least 95% identical to SEQ ID NO:214. The disclosure also provides antibodies comprising a heavy chain variable domain sequence that is at least 80%, at least 85%, at least 90%, or at least 95% identical to SEQ ID NO: 145 and a light chain variable domain sequence that is at least 80%, at least 85%, at least 90%, or at least 95% identical to SEQ ID NO:218. The disclosure also provides antibodies comprising a heavy chain variable domain sequence that is at least 80%, at least 85%, at least 90%, or at least 95% identical to SEQ ID NO:146 and a light chain variable domain sequence that is at least 80%, at least 85%, at least 90%, or at least 95% identical to SEQ ID NO:216. The disclosure also provides antibodies comprising a heavy chain variable domain sequence that is at least 80%, at least 85%, at least 90%, or at least 95% identical to SEQ ID NO: 147 and a light chain variable domain sequence that is at least 80%, at least 85%, at least 90%, or at least 95% identical to SEQ ID NO:286. The disclosure also provides antibodies comprising a heavy chain variable domain sequence that is at least 80%, at least 85%, at least 90%, or at least 95% identical to SEQ ID NO:148 and a light chain variable domain sequence that is at least 80%, at least 85%, at least 90%, or at least 95% identical to SEQ ID NO:295. The disclosure also provides antibodies comprising a heavy chain variable domain sequence that is at least 80%, at least 85%, at least 90%, or at least 95% identical to SEQ ID NO: 149 and a light chain variable domain sequence that is at least 80%, at least 85%, at least 90%, or at least 95% identical to SEQ ID NO:190. The disclosure also provides antibodies comprising a heavy chain variable domain sequence that is at least 80%, at least 85%, at least 90%, or at least 95% identical to SEQ ID NO:150 and a light chain variable domain sequence that is at least 80%, at least 85%, at least 90%, or at least 95% identical to SEQ ID NO: 193. The disclosure also provides antibodies comprising a heavy chain variable domain sequence that is at least 80%, at least 85%, at least 90%, or at least 95% identical to SEQ ID NO: 151 and a light chain variable domain sequence that is at least 80%, at least 85%, at least 90%, or at least 95% identical to SEQ ID NO:194. The disclosure also provides antibodies comprising a heavy chain variable domain sequence that is at least 80%, at least 85%, at least 90%, or at least 95% identical to SEQ ID NO:152 and a light chain variable domain sequence that is at least 80%, at least 85%, at least 90%, or at least 95% identical to SEQ ID NO:297. The disclosure also provides antibodies comprising a heavy chain variable domain sequence that is at least 80%, at least 85%, at least 90%, or at least 95% identical to SEQ ID NO: 153 and a light chain variable domain sequence that is at least 80%, at least 85%, at least 90%, or at least 95% identical to SEQ ID NO:194. The disclosure also provides antibodies comprising a heavy chain variable domain sequence that is at least 80%, at least 85%, at least 90%, or at least 95% identical to SEQ ID NO:154 and a light chain variable domain sequence that is at least 80%, at least 85%, at least 90%, or at least 95% identical to SEQ ID NO:297. The disclosure also provides antibodies comprising a heavy chain variable domain sequence that is at least 80%, at least 85%, at least 90%, or at least 95% identical to SEQ ID NO: 155 and a light chain variable domain sequence that is at least 80%, at least 85%, at least 90%, or at least 95% identical to SEQ ID NO:192. The disclosure also provides antibodies comprising a heavy chain variable domain sequence that is at least 80%, at least 85%, at least 90%, or at least 95% identical to SEQ ID NO:156 and a light chain variable domain sequence that is at least 80%, at least 85%, at least 90%, or at least 95% identical to SEQ ID NO: 188. The disclosure also provides antibodies comprising a heavy chain variable domain sequence that is at least 80%, at least 85%, at least 90%, or at least 95% identical to SEQ ID NO: 156 and a light chain variable domain sequence that is at least 80%, at least 85%, at least 90%, or at least 95% identical to SEQ ID NO:189. The disclosure also provides antibodies comprising a heavy chain variable domain sequence that is at least 80%, at least 85%, at least 90%, or at least 95% identical to SEQ ID NO:156 and a light chain variable domain sequence that is at least 80%, at least 85%, at least 90%, or at least 95% identical to SEQ ID NO:327. The disclosure also provides antibodies comprising a heavy chain variable domain sequence that is at least 80%, at least 85%, at least 90%, or at least 95% identical to SEQ ID NO: 157 and a light chain variable domain sequence that is at least 80%, at least 85%, at least 90%, or at least 95% identical to SEQ ID NO:192. The disclosure also provides antibodies comprising a heavy chain variable domain sequence that is at least 80%, at least 85%, at least 90%, or at least 95% identical to SEQ ID NO:157 and a light chain variable domain sequence that is at least 80%, at least 85%, at least 90%, or at least 95% identical to SEQ ID NO:242. The disclosure also provides antibodies comprising a heavy chain variable domain sequence that is at least 80%, at least 85%, at least 90%, or at least 95% identical to SEQ ID NO: 158 and a light chain variable domain sequence that is at least 80%, at least 85%, at least 90%, or at least 95% identical to SEQ ID NO:192. The disclosure also provides antibodies comprising a heavy chain variable domain sequence that is at least 80%, at least 85%, at least 90%, or at least 95% identical to SEQ ID NO:159 and a light chain variable domain sequence that is at least 80%, at least 85%, at least 90%, or at least 95% identical to SEQ ID NO: 191. The disclosure also provides antibodies comprising a heavy chain variable domain sequence that is at least 80%, at least 85%, at least 90%, or at least 95% identical to SEQ ID NO: 160 and a light chain variable domain sequence that is at least 80%, at least 85%, at least 90%, or at least 95% identical to SEQ ID NO:191. The disclosure also provides antibodies comprising a heavy chain variable domain sequence that is at least 80%, at least 85%, at least 90%, or at least 95% identical to SEQ ID NO:161 and a light chain variable domain sequence that is at least 80%, at least 85%, at least 90%, or at least 95% identical to SEQ ID NO: 192. The disclosure also provides antibodies comprising a heavy chain variable domain sequence that is at least 80%, at least 85%, at least 90%, or at least 95% identical to SEQ ID NO: 162 and a light chain variable domain sequence that is at least 80%, at least 85%, at least 90%, or at least 95% identical to SEQ ID NO:192. The disclosure also provides antibodies comprising a heavy chain variable domain sequence that is at least 80%, at least 85%, at least 90%, or at least 95% identical to SEQ ID NO:163 and a light chain variable domain sequence that is at least 80%, at least 85%, at least 90%, or at least 95% identical to SEQ ID NO:248. The disclosure also provides antibodies comprising a heavy chain variable domain sequence that is at least 80%, at least 85%, at least 90%, or at least 95% identical to SEQ ID NO: 164 and a light chain variable domain sequence that is at least 80%, at least 85%, at least 90%, or at least 95% identical to SEQ ID NO:270. The disclosure also provides antibodies comprising a heavy chain variable domain sequence that is at least 80%, at least 85%, at least 90%, or at least 95% identical to SEQ ID NO:165 and a light chain variable domain sequence that is at least 80%, at least 85%, at least 90%, or at least 95% identical to SEQ ID NO:273. The disclosure also provides antibodies comprising a heavy chain variable domain sequence that is at least 80%, at least 85%, at least 90%, or at least 95% identical to SEQ ID NO: 166 and a light chain variable domain sequence that is at least 80%, at least 85%, at least 90%, or at least 95% identical to SEQ ID NO:281. The disclosure also provides antibodies comprising a heavy chain variable domain sequence that is at least 80%, at least 85%, at least 90%, or at least 95% identical to SEQ ID NO:167 and a light chain variable domain sequence that is at least 80%, at least 85%, at least 90%, or at least 95% identical to SEQ ID NO:322. The disclosure also provides antibodies comprising a heavy chain variable domain sequence that is at least 80%, at least 85%, at least 90%, or at least 95% identical to SEQ ID NO: 168 and a light chain variable domain sequence that is at least 80%, at least 85%, at least 90%, or at least 95% identical to SEQ ID NO:209. The disclosure also provides antibodies comprising a heavy chain variable domain sequence that is at least 80%, at least 85%, at least 90%, or at least 95% identical to SEQ ID NO:169 and a light chain variable domain sequence that is at least 80%, at least 85%, at least 90%, or at least 95% identical to SEQ ID NO:321. The disclosure also provides antibodies comprising a heavy chain variable domain sequence that is at least 80%, at least 85%, at least 90%, or at least 95% identical to SEQ ID NO: 170 and a light chain variable domain sequence that is at least 80%, at least 85%, at least 90%, or at least 95% identical to SEQ ID NO:279. The disclosure also provides antibodies comprising a heavy chain variable domain sequence that is at least 80%, at least 85%, at least 90%, or at least 95% identical to SEQ ID NO:171 and a light chain variable domain sequence that is at least 80%, at least 85%, at least 90%, or at least 95% identical to SEQ ID NO:303. The disclosure also provides antibodies comprising a heavy chain variable domain sequence that is at least 80%, at least 85%, at least 90%, or at least 95% identical to SEQ ID NO: 171 and a light chain variable domain sequence that is at least 80%, at least 85%, at least 90%, or at least 95% identical to SEQ ID NO:323. The disclosure also provides antibodies comprising a heavy chain variable domain sequence that is at least 80%, at least 85%, at least 90%, or at least 95% identical to SEQ ID NO:172 and a light chain variable domain sequence that is at least 80%, at least 85%, at least 90%, or at least 95% identical to SEQ ID NO:207. The disclosure also provides antibodies comprising a heavy chain variable domain sequence that is at least 80%, at least 85%, at least 90%, or at least 95% identical to SEQ ID NO: 173 and a light chain variable domain sequence that is at least 80%, at least 85%, at least 90%, or at least 95% identical to SEQ ID NO:200. The disclosure also provides antibodies comprising a heavy chain variable domain sequence that is at least 80%, at least 85%, at least 90%, or at least 95% identical to SEQ ID NO:174 and a light chain variable domain sequence that is at least 80%, at least 85%, at least 90%, or at least 95% identical to SEQ ID NO:247. The disclosure also provides antibodies comprising a heavy chain variable domain sequence that is at least 80%, at least 85%, at least 90%, or at least 95% identical to SEQ ID NO: 175 and a light chain variable domain sequence that is at least 80%, at least 85%, at least 90%, or at least 95% identical to SEQ ID NO:199. The disclosure also provides antibodies comprising a heavy chain variable domain sequence that is at least 80%, at least 85%, at least 90%, or at least 95% identical to SEQ ID NO:175 and a light chain variable domain sequence that is at least 80%, at least 85%, at least 90%, or at least 95% identical to SEQ ID NO:210. The disclosure also provides antibodies comprising a heavy chain variable domain sequence that is at least 80%, at least 85%, at least 90%, or at least 95% identical to SEQ ID NO: 176 and a light chain variable domain sequence that is at least 80%, at least 85%, at least 90%, or at least 95% identical to SEQ ID NO:196. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:201. The disclosure also provides antibodies comprising a heavy chain variable domain sequence that is at least 80%, at least 85%, at least 90%, or at least 95% identical to SEQ ID NO: 178 and a light chain variable domain sequence that is at least 80%, at least 85%, at least 90%, or at least 95% identical to SEQ ID NO:274. The disclosure also provides antibodies comprising a heavy chain variable domain sequence that is at least 80%, at least 85%, at least 90%, or at least 95% identical to SEQ ID NO:179 and a light chain variable domain sequence that is at least 80%, at least 85%, at least 90%, or at least 95% identical to SEQ ID NO:209. The disclosure also provides antibodies comprising a heavy chain variable domain sequence that is at least 80%, at least 85%, at least 90%, or at least 95% identical to SEQ ID NO: 180 and a light chain variable domain sequence that is at least 80%, at least 85%, at least 90%, or at least 95% identical to SEQ ID NO:209. The disclosure also provides antibodies comprising a heavy chain variable domain sequence that is at least 80%, at least 85%, at least 90%, or at least 95% identical to SEQ ID NO:181 and a light chain variable domain sequence that is at least 80%, at least 85%, at least 90%, or at least 95% identical to SEQ ID NO:208. The disclosure also provides antibodies comprising a heavy chain variable domain sequence that is at least 80%, at least 85%, at least 90%, or at least 95% identical to SEQ ID NO: 182 and a light chain variable domain sequence that is at least 80%, at least 85%, at least 90%, or at least 95% identical to SEQ ID NO:253. The disclosure also provides antibodies comprising a heavy chain variable domain sequence that is at least 80%, at least 85%, at least 90%, or at least 95% identical to SEQ ID NO:183 and a light chain variable domain sequence that is at least 80%, at least 85%, at least 90%, or at least 95% identical to SEQ ID NO:276. The disclosure also provides antibodies comprising a heavy chain variable domain sequence that is at least 80%, at least 85%, at least 90%, or at least 95% identical to SEQ ID NO: 184 and a light chain variable domain sequence that is at least 80%, at least 85%, at least 90%, or at least 95% identical to SEQ ID NO:275. The disclosure also provides antibodies comprising a heavy chain variable domain sequence that is at least 80%, at least 85%, at least 90%, or at least 95% identical to SEQ ID NO:185 and a light chain variable domain sequence that is at least 80%, at least 85%, at least 90%, or at least 95% identical to SEQ ID NO:293. The disclosure also provides antibodies comprising a heavy chain variable domain sequence that is at least 80%, at least 85%, at least 90%, or at least 95% identical to SEQ ID NO: 186 and a light chain variable domain sequence that is at least 80%, at least 85%, at least 90%, or at least 95% identical to SEQ ID NO:226. The disclosure also provides antibodies comprising a heavy chain variable domain sequence that is at least 80%, at least 85%, at least 90%, or at least 95% identical to SEQ ID NO:186 and a light chain variable domain sequence that is at least 80%, at least 85%, at least 90%, or at least 95% identical to SEQ ID NO:258.

Table 1 provides a summary of the TIM-3-specific antibodies described herein:

### Pharmaceutical Compositions

Pharmaceutical compositions comprising the antibodies or antibody fragments of the present disclosure are also contemplated and can be used in the methods disclosed herein. Pharmaceutical compositions can comprise one or more of the antibodies or antibody fragments described herein and a pharmaceutically acceptable carrier or excipient. Although the carrier or excipient may facilitate administration, it should not itself induce the production of antibodies harmful to the subject or individual receiving the composition; nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles, and are known to one of skill in the art.

The antibodies or an antigen binding fragments described herein, or the pharmaceutical compositions disclosed herein, may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Typically, the therapeutic compositions may be prepared as injectables, either as liquid solutions or suspensions. Solid forms suitable for solution in, or suspension in, liquid vehicles prior to injection may also be prepared.

In one embodiment, the antibody, or an antigen binding fragment thereof, or pharmaceutical composition is administered intravenously. In another embodiment, the antibody, or an antigen binding fragment thereof, or pharmaceutical composition is administered by intravenous infusion.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g., whether a pharmaceutical should be delivered daily, weekly, monthly, etc. Frequency and dosage may also depend on the severity of symptoms.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation, but which release the antibody once it has been absorbed from the gastrointestinal tract.

The methods of the present invention can use an antibody, or an antigen binding fragment thereof, as described above, alone or in combination with other pharmaceutically active compounds, to treat conditions such as those disclosed hereinabove. The additional pharmaceutically active compound(s) can be administered simultaneously (either in the same dosage form or in separate dosage forms) or sequentially. Accordingly, in one embodiment, the present invention comprises methods for treating a condition by administering to the subject a therapeutically effective amount of an antibody, or an antigen binding fragment thereof, of the present invention and one or more additional pharmaceutically active compounds.

In some embodiments, the antibody, or an antigen binding fragment thereof, of the present invention is used in combination with existing TIM-3-related disease therapies.

### EXAMPLES

The following examples put forth so as to provide those of ordinary skill in the art with a complete description of how to make and use the present disclosure, and is not intended to limit the scope of what the inventors regard as their disclosure nor is it intended to represent that the experiment below is all or the only experiment that could be performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Isolation of Murine Anti-Human TIM-3 Antibodies

### Immunization, & Single Cell Suspension Generation

Recombinant human TIM-3 His tagged, catalog no: TM3-H5229, ACROBiosystems, Beijing, China, SEQ ID NO:2) was used to immunize young C57/BL6 mice each with 80 µg of the protein in Sigma Adjuvant System® (Sigma-Aldrich, St. Louis, MO) over a period of 35 days using a rapid immunization protocol of Antibody Solutions (Sunnyvale, CA). The lymph nodes were harvested on day 35. Single cell suspension of the lymph node was generated, and the suspension was filtered through a 70 µm mesh (BD Bioscience) to remove clumps.

### Plasma Cell Isolation, Antibody Capture, and Antigen Interrogation

The filtered lymphocyte suspension was enriched for plasma cells actively secreting IgGs using a kit based on cell surface expression of CD138 (Miltenyi, Auburn, CA). Using a method described in U.S. Pat. No. 9,328,172, freshly enriched plasma cells were deposited on a PDMS device to allow a single cell settled in the microwells on the device. Antibody secreted from each plasma cell was captured on a derivatized microscope slide. Antigen-specific antibody secreting cells were identified by interrogating the antibody capture slide with varying concentrations of fluorescently labeled TIM-3 protein tagged with human Fc (ACROBiosystems, Beijing, China, catalog no: TM3-H5258, SEQ ID NO:3). Labeling was done using a kit (AnaSpec, Fremont, CA, AS-72046, AnaTag™ HiLyte™ Fluor 555 Microscale Protein Labeling Kit *Ultra Convenient*).

### mRNA Capture

After antibody capture, the medium was removed, and replaced with lysis buffer followed by prompt closure of the top of the microwells with a custom oligonucleotide microarray (Agilent, Santa Clara, CA). This procedure was previously described in U.S. Pat. No. 9,328,172. The custom oligonucleotide microarray is prepared such that each feature contains not only a unique tag specifying its coordinate but also capture probes for all subclasses (1, 2a, 2b, and 3) of murine IgG heavy chain, murine Ig kappa light chain.

### cDNA Synthesis, PCR Amplification, and Next Generation Sequencing

Captured mRNA on the custom microarray was further processed to synthesize cDNA of each mRNA incorporating the unique tag originally on each feature. The cDNA is then amplify using a Taq polymerase (Promega, Madison, WI) and appropriate set of primers to allow amplification of the following genes: variable domain of IgG heavy chain subclasses and variable domain of Ig kappa light chain. Though now released from cells, these fragments of each gene are now labeled with the unique tag from the custom oligonucleotide microarray manifesting their originating locations. The amplicons were further manipulated to have appropriate sequence attached at both ends to enable sequencing on an Illumina MiSeq instrument using 2 x 250 bp chemistry at SeqMatic LLC (Fremont, CA).

### Bioinformatic Analysis of Images and DNA Sequences

Sequencing reads from MiSeq were processed and the embedded tag in each read was identified and converted into coordinates. The coordinates were plotted to yield a synthetic map of the mRNA recovered. Most of the coordinates form clusters that designate the location of the originating cell for the recovered mRNA sequences. Next, CDR3 motif present in each read with the coordinates was identified and collated according to the clusters that matched the location of an antibody spot visualized by an appropriate fluorescently labeled secondary antibody. Identical or nearly identical CDR3s for a given antibody spot were organized and form consistent pair of V_{H} and V_{L} sequences. The remaining part of V_{H} or V_{L} sequence containing the identified CDR3s was identified and the associated sequencing reads were assembled into full-length cDNA sequences for V_{H} and V_{L}. The pair of full-length cDNA was correlated with the affinity measurements associated with each of the antigen-specific antibody spot.

### Example 2: Molecular Reconstruction and Recombinant Expression of Anti-TIM-3 Antibodies

A select subset of paired V_{H} and V_{L} anti-TIM-3 antibody sequences was used to synthesize corresponding gene fragments by a service provider according to the known art. The resulting gene fragments were cloned into an appropriate plasmid vector and transfected into an appropriate mammalian host, such as HEK293, for recombinant expression to produce an antibody preparation in full-IgG format. The antibody preparations were characterized by measurements at OD280 to assess the amount produced and by gel electrophoresis on PAGE to assess the size of the antibody chains produced.

### Example 3: Characterization of Recombinant Anti-TIM-3 Antibodies

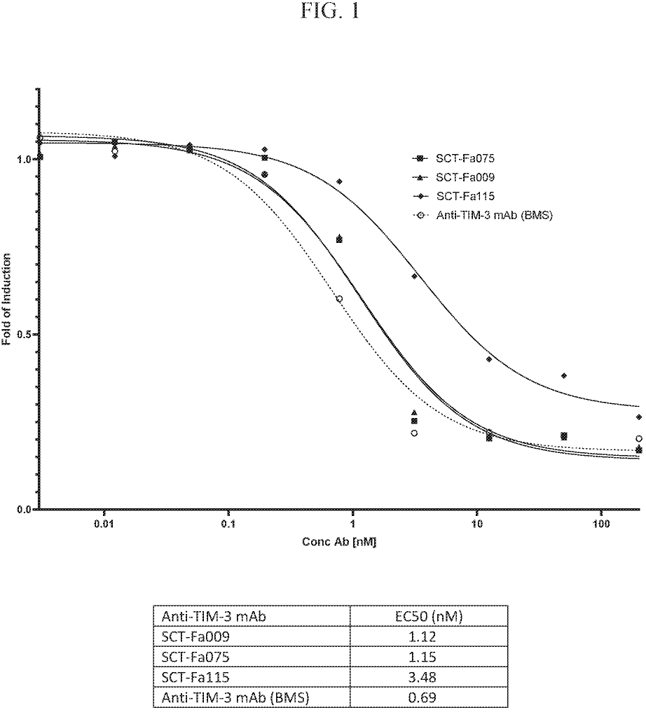

The recombinantly expressed antibodies were used to assess their ability to block TIM-3 signaling via the phosphatidylserine axis using a reporter cell assay. Such an assay as a kit is commercially available (Cat. No. JA2211, Promega, Madison, WI). The kit contains TIM-3 effector cells by genetically engineered to express human TIM-3 and a NanoLuc luciferase reporter drive by T cell activation pathway-dependent response elements. A corresponding target cells, MHCII-positive human cell line is also included. Once set up, the resulting luminescence signals are detected by a luminometer (CS1978C14, Promega, Madison, WI) and the results, including the EC50 values, will be used to compare against benchmark antibodies with known TIM-3 bioactivity (FIG. 1). Blocking antibodies will show reduced luminescence as a result of blocking TIM-3 signaling in a dose-dependent manner.

Antibodies recovered from the antibody campaign described above are listed herein. The CDR sequences and the V_{H} and V_{L} sequences for the anti-TIM-3 antibodies described herein are depicted in Tables 2 and 3, respectively.

TABLE 2

CDR sequences of mAbs generated against human TIM-3 (SEQ ID Nos for each listed sequence are provided in parenthesis)

| mAb AA ID | HCDR1 | HCDR2 | HCDR3 | LCDR1 | LCDR2 | LCDR3 |
|---|---|---|---|---|---|---|
| SCT-Fa001 | GNSFTDYN | INPNYGTT | AREGNG | QSLLDSDGKTY | LVS | WQGTLFPWT |
| | (333) | (334) | (335) | (336) | (337) | (338) |
| SCT-Fa002 | GYSFTDYN | NNPKYGTT | ARRGYYYGSSYGYFDV | QDIRSN | HGT | VQYVQFPRT |
| | (339) | (340) | (341) | (342) | (343) | (344) |
| SCT-Fa003 | GFTFSDAW | IRNKANKH | SYDYIFAY | QSLVHSNGNTY | KVS | SQSTHVPWT |
| | (345) | (346) | (347) | (348) | (349) | (350) |
| SCT-Fa004 | GFNIKGHY | IDPDDGAT | TRSNPYYFDY | QNINVW | KAS | QQGQNYPYT |
| | (351) | (352) | (353) | (354) | (355) | (356) |
| SCT-Fa005 | GFNNKNTY | IDPANGNV | ARGSSRLYYALDY | ESVDSYGNSF | RAS | QQSNEDPYT |
| | (357) | (358) | (359) | (360) | (361) | (362) |
| SCT-Fa006 | GFTFSDYG | ISSGSISS | ARHDWRGLYGMDY | SSVSSSY | STS | HQWSSYPPT |
| | (363) | (364) | (365) | (366) | (367) | (368) |
| SCT-Fa007 | GFTFSDYY | INFDGSGT | ARLYDGYYAY | QSLVHSNGNTY | KVS | SQSTHVPLT |
| | (369) | (370) | (371) | (348) | (349) | (372) |
| SCT-Fa008 | GFTFSDYY | INFDGSGT | ARLYDGYYAY | QSLVHSNGNTY | KVS | SQSTHVPLT |
| | (369) | (370) | (371) | (348) | (349) | (372) |
| SCT-Fa009 | GFTFSDYY | INYDGSRT | ARNIYYYGGSSYGMDY | GNIHNY | NAE | QHFWSTPLT |
| | (369) | (373) | (374) | (375) | (376) | (377) |
| SCT-Fa010 | GFTFSDYY | INFDGSGT | ARLYDGSLDY | QSLLYTNGKTY | LVS | VQGTHFPLT |
| | (369) | (370) | (378) | (379) | (337) | (380) |
| SCT-Fa011 | GFTFSDYY | INFDGSGT | ARLYDGSHDY | QSLLYSNGKTY | LVS | VQGTHFPLT |
| | (369) | (370) | (381) | (382) | (337) | (380) |
| SCT-Fa012 | GFTFSDYY | INFDGSGT | ARLYDGSHDY | QSLLYSNGKTY | LVS | VQGTHFPLT |
| | (369) | (370) | (381) | (382) | (337) | (380) |
| SCT-Fa013 | GFTFSDYY | INFDGSGT | ARLYDGSHDY | QSLLYSNGKTY | LVS | VQGTHFPLT |
| | (369) | (370) | (381) | (382) | (337) | (380) |
| SCT-Fa014 | GFTFSDYY | INFDGSGT | ARLYDGSHDY | QSLLYSNGKTY | LVS | VQGTHFPLT |
| | (369) | (370) | (381) | (382) | (337) | (380) |
| SCT-Fa015 | GFTFSDYY | INFDGSGT | ARLYDGSHDY | QSLLYSNGKTY | LVS | VQGTHFPLT |
| | (369) | (370) | (381) | (382) | (337) | (380) |
| SCT-Fa016 | GFTFSDYY | INFDGSGT | ARLYDGSHDY | QSLLYSNGKTY | LVS | VQGTHFPLT |
| | (369) | (370) | (381) | (382) | (337) | (380) |
| SCT-Fa017 | GFTFSDYY | INFDGSGT | ARLYDGSHDY | QSLLYSNGKTY | LVS | VQGTHFPQT |
| | (369) | (370) | (381) | (382) | (337) | (383) |
| SCT-Fa018 | GFTFSDYY | INFDGSSS | ARLYDGYYAY | QSLIHSNGNTY | KVS | SQSTHVPLT |
| | (369) | (384) | (371) | (385) | (349) | (372) |
| SCT-Fa019 | GFTFSDYY | INFDGSGT | ARLYDGSHDY | QSLLYSNGKTY | LVS | VQATHFPLT |
| | (369) | (370) | (381) | (382) | (337) | (386) |
| SCT-Fa020 | GYTFTDYN | INPNNGGI | VGSLDY | QDINNY | YTS | QQGYMLPLT |
| | (387) | (388) | (389) | (390) | (391) | (392) |
| SCT-Fa021 | EYTFADYY | INPYNGGA | ARDAD FYGN SYVGYAMDY | QSINTD | YAS | QQSHTWPQYT |
| | (393) | (394) | (395) | (396) | (397) | (398) |
| SCT-Fa022 | GYTFTSYW | IDPNSGGT | TKDGNTPYYAMDY | QSVSNA | YAS | QQDYSSPLT |
| | (399) | (400) | (401) | (402) | (397) | (403) |
| SCT-Fa023 | DFNIKDYY | IDPDDGET | SRSNPYYFDY | QNINVW | KAS | QQGQNYPYT |
| | (404) | (405) | (406) | (354) | (355) | (356) |
| SCT-Fa024 | DFNIKDYY | IDPEDDET | ARSNPYYFDY | QNINVW | KAS | QQGQNYPYT |
| | (404) | (407) | (408) | (354) | (355) | (356) |
| SCT-Fa025 | GFNIKDFY | IDPEDGKT | ARSNPYFFDY | QTINVW | KAS | HQGQSYPYT |
| | (409) | (410) | (411) | (412) | (355) | (413) |
| SCT-Fa026 | GFNIKDFY | IDPEDGKT | ARSNPYFFDY | QTINVW | KAS | HQGQSYPYT |
| | (409) | (410) | (411) | (412) | (355) | (413) |
| SCT-Fa027 | GFNIKDYY | IDPDNGET | ARSSPYYFDY | QNINVW | KAS | QQGQNFPYT |
| | (414) | (415) | (416) | (354) | (355) | (417) |
| SCT-Fa028 | GFNIKDYY | IDPDNGET | ARSSPYYFDY | QNINVW | KAS | QQGQNFPYT |
| | (414) | (415) | (416) | (354) | (355) | (417) |
| SCT-Fa029 | GFNIKDYY | IDPEDGKT | ARSNPYYFDY | QNINVW | KAS | QQGQSYPYT |
| | (414) | (410) | (408) | (354) | (355) | (418) |
| SCT-Fa030 | GFNIKDYY | IDPEDGET | ARSNPYFFDY | QTINVW | KAS | HQGQSYPYT |
| | (414) | (419) | (411) | (412) | (355) | (413) |
| SCT-Fa031 | GFNIKDYY | IDPEDGET | ARSNPYYFDY | QTINVW | KAS | QQGQNYPYT |
| | (414) | (419) | (408) | (412) | (355) | (356) |
| SCT-Fa032 | GFNIKDYY | IDPEDGET | ARSNPYYFDY | QNINVW | KAS | QQGQNYPYT |
| | (414) | (419) | (408) | (354) | (355) | (356) |
| SCT-Fa033 | GFNIKDYY | IDPEDGET | GRSNPYYFDY | QNINVW | KAS | QQGQNYPYT |
| | (414) | (419) | (420) | (354) | (355) | (356) |
| SCT-Fa034 | GFNIKDYY | IDPEDGET | ARSNPYYFDY | QTINVW | KAS | QQGQNYPYT |
| | (414) | (419) | (408) | (412) | (355) | (356) |
| SCT-Fa035 | GFNIKDYY | IDPEDGET | ARSNPYYFDY | QSLLDSDGKTY | LVS | WQGTLFPWT |
| | (414) | (419) | (408) | (336) | (337) | (338) |
| SCT-Fa036 | DFNIKDYY | IDPEDGET | ARSNPYYFDY | QNVGTN | SAS | QQYNTYPLT |
| | (404) | (419) | (408) | (421) | (422) | (423) |
| SCT-Fa037 | GFNIKDYY | IDPDNDKI | ARSNPYFFDY | QTINIW | KAS | HQGQSYPYT |
| | (414) | (424) | (411) | (425) | (355) | (413) |
| SCT-Fa038 | GFNIKDYY | IDPENDKI | ARSNPYFFDY | QTINVW | KAS | HQGQSYPYT |
| | (414) | (426) | (411) | (412) | (355) | (413) |
| SCT-Fa039 | DFNIKDYY | IDPEDGET | TRSNPYYFDY | QNINVW | KAS | QQGQNFPYT |
| | (404) | (419) | (353) | (354) | (355) | (417) |
| SCT-Fa040 | GFNIKDYY | IDPEDGET | TRSNPYYFDY | QNINVW | KAS | QQGQNFPYT |
| | (414) | (419) | (353) | (354) | (355) | (417) |
| SCT-Fa041 | GFNIKDYY | IDPDNGET | ARSSPYYFDY | QNINVW | KAS | QQGQNFPYT |
| | (414) | (415) | (416) | (354) | (355) | (417) |
| SCT-Fa042 | GFNIKDYY | IDPDNGET | ARSSPYYFDY | QNINVW | KAS | QQGQNFPYT |
| | (414) | (415) | (416) | (354) | (355) | (417) |
| SCT-Fa043 | GFNIKDYY | IDPDNDEI | ARSSPYYFDY | QNINVW | KAS | QQGQNIPYT |
| | (414) | (427) | (416) | (354) | (355) | (428) |
| SCT-Fa044 | GFNIKDYY | IDPDNDEI | ARSSPYYFDY | QNINVW | KAS | QQGQNIPYT |
| | (414) | (427) | (416) | (354) | (355) | (428) |
| SCT-Fa045 | GFNIKDYY | IDPDDAET | TRDFGYTDYFDY | QDVSTA | SAS | QQHYSTPT |
| | (414) | (429) | (430) | (431) | (422) | (432) |
| SCT-Fa046 | GYTFTDYN | IYPYNGGT | AREDYGSREWFAY | QSLVHSNGNTY | KVS | SQSTHVPLT |
| | (387) | (433) | (434) | (348) | (349) | (372) |
| SCT-Fa047 | GYTFTDYY | INLNNGGT | EGLGGY | QSIVHSNGNTY | KVA | FQGSHVPFT |
| | (435) | (436) | (437) | (438) | (439) | (440) |
| SCT-Fa048 | GYTLTDYY | INPNNGDI | EGLGGY | QTIVHSNGNTY | KVS | FQGSHVPFT |
| | (441) | (442) | (437) | (443) | (349) | (440) |
| SCT-Fa049 | GYTLTDYY | INPNNGDI | EGLGGY | QTIVHSNGNTY | KVS | FQGSHVPFT |
| | (441) | (442) | (437) | (443) | (349) | (440) |
| SCT-Fa050 | GNTFTAFY | VNPYNGGA | LLWEFAH | QSLLDSDGKTY | LVS | WQGTHSWT |
| | (444) | (445) | (446) | (336) | (337) | (447) |
| SCT-Fa051 | EYTFTDYY | INPYNGGA | ARDVDFYGNRYVGYAMDY | QSISNN | YAS | QQSNSWPQYT |
| | (448) | (394) | (449) | (450) | (397) | (451) |
| SCT-Fa052 | GYSFTGYH | INPYNSAS | VRGAYGNPLDY | QDINSY | RTN | LQYDEYPYT |
| | (452) | (453) | (454) | (455) | (456) | (457) |
| SCT-Fa053 | GYSFTGYH | INPYNSAT | VRGAYGNPLDY | QDINSY | RAN | LQYDEYPYT |
| | (452) | (458) | (454) | (455) | (459) | (457) |
| SCT-Fa054 | GYSFTGYH | INPYNSAT | VRGAYGNPLDY | QDINSY | RAN | LQYDEYPYT |
| | (452) | (458) | (454) | (455) | (459) | (457) |
| SCT-Fa055 | GYTFTDYH | ISPYNGGT | ARSDYGSPLDY | QDINSY | RAN | LQYDEFPYT |
| | (460) | (461) | (462) | (455) | (459) | (463) |
| SCT-Fa056 | GYTFTDYY | INPYNGGA | ARDVDFYDNAYVGYAMDY | QTISNN | YAS | QQSNSWPQYT |
| | (435) | (394) | (464) | (465) | (397) | (451) |
| SCT-Fa057 | GYTFTDYY | INPYNGGT | ARGGDYDVNYSAMDY | QSLLDSDGKTY | LVS | WQGTHFPTWT |
| | (435) | (466) | (467) | (336) | (337) | (468) |
| SCT-Fa058 | EYTFTDYY | INPYNGGV | ARDADFHGNSYVGYAMDY | QSISND | YAS | QQSHSWPQYT |
| | (448) | (469) | (470) | (471) | (397) | (472) |
| SCT-Fa059 | GYSFTGYH | INPYNSGI | VRGAYGNPLDY | QDINSY | RTN | LQYDEYPYT |
| | (452) | (473) | (454) | (455) | (456) | (457) |
| SCT-Fa060 | GYTFTDYY | INPYNGGT | ARDVDYFDNKYVGYAMDY | QSISNN | YTS | QQSNSWPQYT |
| | (435) | (466) | (474) | (450) | (391) | (451) |
| SCT-Fa061 | GYSFTGYH | INPYNSAT | VRGAYGNPLDY | QDINSY | RAN | LQYDEYPYT |
| | (452) | (458) | (454) | (455) | (459) | (457) |
| SCT-Fa062 | GYTFTRYW | IYPGNSDT | TFYYGNYVNY | QSLLDSDGKTY | LVS | WQGTHFPTWT |
| | (475) | (476) | (477) | (336) | (337) | (468) |
| SCT-Fa063 | GYTFIRYW | IYPGNSDT | TFYYGNYVNF | QSLLDSDGKTY | LVS | WQGTHFPTWT |
| | (478) | (476) | (479) | (336) | (337) | (468) |
| SCT-Fa064 | GYTFTRYW | IYPGNSDT | TFFYGNYVNY | QSLLYSSNQKNY | WAS | QQYYSYPRT |
| | (475) | (476) | (480) | (481) | (482) | (483) |
| SCT-Fa065 | GYTFTRYW | IYPGNSDT | TFYYGNYVNY | QSLLDSDGKTY | LVS | WQGTHFPTWT |
| | (475) | (476) | (477) | (336) | (337) | (468) |
| SCT-Fa066 | GYTFTRYW | IYPGNSDT | TFYYGNYVNY | QSLLASDGKTY | LVS | WQGTHFPTWT |
| | (475) | (476) | (477) | (484) | (337) | (468) |
| SCT-Fa067 | GYTFTRYW | IYPGNSDT | TFYYGNYVNY | QSLLDSDGKTY | LVS | WQGTHFPTWT |
| | (475) | (476) | (477) | (336) | (337) | (468) |
| SCT-Fa068 | GYTFTRYW | IYPGNSDT | TFYYGNYVNY | QSLLDSDGKTY | LVS | WQGTHFPTWT |
| | (475) | (476) | (477) | (336) | (337) | (468) |
| SCT-Fa069 | GYTFTRYW | IYPGNSDT | TFFYGNYVNY | QSLLDSDGKTY | LVS | WQGTHFPTWT |
| | (475) | (476) | (480) | (336) | (337) | (468) |
| SCT-Fa070 | GYTFTRYW | IYPGNSDT | TFYYGNYVNY | QSLLDSDGKTY | LVS | WQGTHFPTWT |
| | (475) | (476) | (477) | (336) | (337) | (468) |
| SCT-Fa071 | GFNIKDFY | IDPEDGKT | ARSNPYFFDY | QNVGTN | SAS | QQYNSYPYT |
| | (409) | (410) | (411) | (421) | (422) | (485) |
| SCT-Fa072 | GFTFSDYG | ISSGGSYT | TRHEITTVLSTDYAMDY | QSLVHSIGNTY | KVS | SQSTHIPWT |
| | (363) | (486) | (487) | (488) | (349) | (489) |
| SCT-Fa073 | GFTFSDYG | ISSGSISS | ARHDWRGLYAMDY | SSVSSSY | STS | HQSSSYPPT |
| | (363) | (364) | (490) | (366) | (367) | (491) |
| SCT-Fa074 | GFTFSDYG | ISGGSISS | ARHDWRGLYAMDY | SSVSSSY | STS | LQWSSYPPT |
| | (363) | (492) | (490) | (366) | (367) | (493) |
| SCT-Fa075 | GFTFSDYG | ISGGSISS | ARHDWRGLYAMDY | SSVSSSY | STS | LQWSSYPPT |
| | (363) | (492) | (490) | (366) | (367) | (493) |
| SCT-Fa076 | GFTFSGFW | INSDGSTI | MSLLLNV | QEISGY | RAS | LQYANYPLT |
| | (494) | (495) | (496) | (497) | (361) | (498) |
| SCT-Fa077 | GFTFSDYY | INYDGTNT | GRGIYYYGRSHAMDY | QGINNN | HGT | VQYDQFPPT |
| | (369) | (499) | (500) | (501) | (343) | (502) |
| SCT-Fa078 | GYTFTSYW | IDPSDNYT | AREGAFSSPFAY | QDIRSN | HGT | VQYVQFPRT |
| | (399) | (503) | (504) | (342) | (343) | (344) |
| SCT-Fa079 | GYTFTSYW | IDPSDNYT | AREGAFSSPFAY | QDIRSN | HGT | VQYVQFPRT |
| | (399) | (503) | (504) | (342) | (343) | (344) |
| SCT-Fa080 | GYTFTSYW | IDPSDNYT | AREGAFSSPFAY | QDIRSN | HGT | VQYVQFPRT |
| | (399) | (503) | (504) | (342) | (343) | (344) |
| SCT-Fa081 | GYTFTSYW | IDPSDNYT | AKEGAFSSPFAY | QDIRSN | HGT | VQYVQFPRT |
| | (399) | (503) | (505) | (342) | (343) | (344) |
| SCT-Fa082 | GYTFTSYW | IDPSDNYT | AREGAFSSPFAY | QDIRSN | HGT | VQYVQFPRT |
| | (399) | (503) | (504) | (342) | (343) | (344) |
| SCT-Fa083 | GYTFTSYW | IDPSDNYT | AREGAFSSPFAY | QDIRSN | HGT | VQYIQFPRT |
| | (399) | (503) | (504) | (342) | (343) | (506) |
| SCT-Fa084 | GYTFTSYW | IDPSDNYT | AREGAFSSPFAY | QDIRSN | HGT | VQYVQFPRT |
| | (399) | (503) | (504) | (342) | (343) | (344) |
| SCT-Fa085 | GYTFTDYY | IYPGSDNT | ARSGNYYAMDY | QSVSNN | YAS | QQDYSSPYT |
| | (435) | (507) | (508) | (509) | (397) | (510) |
| SCT-Fa086 | GYTFTDYY | IYPGSGNT | ARAYDYGFPY | QSMNNG | YAS | QQDYSSPYT |
| | (435) | (511) | (512) | (513) | (397) | (510) |
| SCT-Fa087 | GYTFTDYY | IYPGSGNT | ARAYDYGFAY | QSVSYG | YSS | QQDYSSPYT |
| | (435) | (511) | (514) | (515) | (516) | (510) |
| SCT-Fa088 | GYTFTDYY | IYPGSGNT | ARAYDYGFAY | QSVSHG | YAS | QQDYSSPYT |
| | (435) | (511) | (514) | (517) | (397) | (510) |
| SCT-Fa089 | GYTFTDYY | IYPGSGNT | ARSGNYYAMDY | QSVSNN | YAS | QQDYSSPYT |
| | (435) | (511) | (508) | (509) | (397) | (510) |
| SCT-Fa090 | GYIFRTYG | INTYSGEP | ARAQHYFDW | QDIDSY | RAT | LQYDEFPYT |
| | (518) | (519) | (520) | (521) | (522) | (463) |
| SCT-Fa091 | GYTFRTYG | INTYSGEP | ARAQHYFDW | QDIDRY | RAT | LQYDEFPYT |
| | (523) | (519) | (520) | (524) | (522) | (463) |
| SCT-Fa092 | GYTFRTYG | INTYSGEP | ARAQHYFDW | QDIDSY | RAT | LQYDEFPYT |
| | (523) | (519) | (520) | (521) | (522) | (463) |
| SCT-Fa093 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa094 | GYTFTNFW | IDPSDIST | ARNDYDGGYFDY | QSLVHGNGNTY | TVS | SQTIHAPWT |
| | (528) | (529) | (530) | (531) | (532) | (533) |
| SCT-Fa095 | GFSLTSSV | IWAGGDT | ARNLNAMDY | QSIVHSNGNTY | KVS | FQGSHVPWT |
| | (534) | (535) | (536) | (438) | (349) | (537) |
| SCT-Fa096 | GYTFTDFY | IYPGGANT | ARGANWAFAS | QGVSTA | SAS | QQYYNTPYT |
| | (538) | (539) | (540) | (541) | (422) | (542) |
| SCT-Fa097 | GYILTDYY | IYPGNGNT | ARGGSSDRIYYYAMDH | QNINVW | KAS | QQGQSYPLT |
| | (543) | (525) | (544) | (354) | (355) | (527) |
| SCT-Fa098 | GYILTDYY | IYPGNGNT | ARGGSSDRIYYYAMDY | QNINVW | KAS | QQGQSYPLT |
| | (543) | (525) | (526) | (354) | (355) | (527) |
| SCT-Fa099 | GYILTDYY | IYPGNGNT | ARGGSSDRIYYYAMDY | QNINVW | KAS | QQGQSYPLT |
| | (543) | (525) | (526) | (354) | (355) | (527) |
| SCT-Fa100 | GYTLIDYY | IYPGNGNT | ARGGSSDRIYYFAMDY | QNINVW | KAS | QQGQSYPLT |
| | (545) | (525) | (546) | (354) | (355) | (527) |
| SCT-Fa101 | GYTLIDYY | IYPGNGNT | ARGGSSDRIYYYAMDY | QNINVW | KAS | QQGQSYPLT |
| | (545) | (525) | (526) | (354) | (355) | (527) |
| SCT-Fa102 | GYTLNDYY | IYPGNNNT | ARGGSSVRVYYYAMDY | QNINVW | KAS | QQGQSYPLT |
| | (547) | (548) | (549) | (354) | (355) | (527) |
| SCT-Fa103 | GYTLTDYY | IYPGNGNT | ARGGSSVRVYYYAMDF | QNINVW | KAS | QQGQSYPLT |
| | (441) | (525) | (550) | (354) | (355) | (527) |
| SCT-Fa104 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa105 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa106 | GYTLTDYY | IYPGNGNT | ARGGSSVRVYYYAMDY | QNINVW | KAS | QQGQSYPLT |
| | (441) | (525) | (549) | (354) | (355) | (527) |
| SCT-Fa107 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa108 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa109 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa110 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa111 | GFSLISYG | IWSGGNTD | ARTANALDY | QTIVHSNGNTY | KVS | FQGSHVPWT |
| | (551) | (552) | (553) | (443) | (349) | (537) |
| SCT-Fa112 | GYTFTNYW | IDPSDNYT | AGEGAFSSPFAY | QDIRSN | HGT | VQYVQFPRT |
| | (554) | (503) | (555) | (342) | (343) | (344) |
| SCT-Fa113 | GNTFTSYW | IDPSDSYI | AREGAYSSPFAY | QDIRSN | HGS | VQYIQFPRT |
| | (556) | (557) | (558) | (342) | (559) | (506) |
| SCT-Fa114 | GYTFTSYW | IDPDSGGT | ARYYWFFDI | QDVSTA | SAS | QQHFNTPFT |
| | (399) | (560) | (561) | (431) | (422) | (562) |
| SCT-Fa115 | CYTFISYW | IDPDSGGT | ARYYWFFDI | QDVGAA | SAS | QQHFNTPFT |
| | (563) | (560) | (561) | (564) | (422) | (562) |
| SCT-Fa116 | GYSFISSW | IHPSDSDT | AIIHRGFYFAMDY | QSIVHSNGNTY | KVS | FQGSHVPWT |
| | (565) | (566) | (567) | (438) | (349) | (537) |
| SCT-Fa117 | GYTFISYW | IDPDSGGT | ARYYWFFDI | QDVGAA | SAS | QQHFNTPFT |
| | (568) | (560) | (561) | (564) | (422) | (562) |
| SCT-Fa118 | GYTFISYW | IDPNSGDT | ARWGWDAFAY | QSLENSDGNTY | RVS | LQVTHVPPT |
| | (568) | (569) | (570) | (571) | (572) | (573) |
| SCT-Fa119 | GYSFISSW | IHPSDSDT | AIIHRGFYFAMDY | QSIVHSNGNTY | KVS | FQGSHVPWT |
| | (565) | (566) | (567) | (438) | (349) | (537) |
| SCT-Fa120 | GYSFISYW | IHPSDSDT | TISHYSGSSPETY | QSIVHSNGNTY | KVS | FQGSHVPYT |
| | (574) | (566) | (575) | (438) | (349) | (576) |
| SCT-Fa121 | GYTFISHW | IHPSDSDT | AIWSYYYGSRLDY | QNIVHSNGNTY | KVS | QGSHVPYT |
| | (577) | (566) | (578) | (579) | (349) | (580) |
| SCT-Fa122 | GYTFTTFW | IDPSDSYT | ARNYGSNYVGYFDY | QDISNY | YTS | QQANALPWT |
| | (581) | (582) | (583) | (584) | (391) | (585) |
| SCT-Fa123 | GYTFNTYW | IDPSDSYT | ARNYGSNYVGYFDY | QDISNY | YTS | QQAKALPWT |
| | (586) | (582) | (583) | (584) | (391) | (587) |
| SCT-Fa124 | GYTFSTYW | IDPSDSYT | ARNYGSNYVGYFDH | QDISNY | YTS | QQGNALPWT |
| | (588) | (582) | (589) | (584) | (391) | (590) |
| SCT-Fa125 | GYTFTSYW | IDPSDNYT | ARNYGSNYVGYFDY | QGISNY | YTS | QQGNSLPWT |
| | (399) | (503) | (583) | (591) | (391) | (592) |
| SCT-Fa126 | GYTFTSYW | IDPSDSYS | ARNYGSNYVGYFDY | QDISNY | YTS | QQGNTLPWT |
| | (399) | (593) | (583) | (584) | (391) | (594) |
| SCT-Fa127 | GYTFTTYW | IDPSDGYS | ARNYGSNYVGYFDY | QDISNY | YTS | QQANALPWT |
| | (595) | (596) | (583) | (584) | (391) | (585) |
| SCT-Fa128 | GYTFTTYW | IDPSDSYS | ARNYGSNYVGYFDY | QDISNY | YTS | QQGKALPWT |
| | (595) | (593) | (583) | (584) | (391) | (597) |
| SCT-Fa129 | GYTFTTYW | IDPSDSYT | ARNYGSNYVGYFDY | QDISNY | YTS | QQGKALPWT |
| | (595) | (582) | (583) | (584) | (391) | (597) |
| SCT-Fa130 | GYTFTTYW | IDPSDSYT | ARNYGSNYVGYFDH | QDISNY | YTS | QQANALPWT |
| | (595) | (582) | (589) | (584) | (391) | (585) |
| SCT-Fa131 | GYTFSTYW | IDPSDSYT | ARNYGSNYVGYFDY | QDINNY | YTS | QQGNALPWT |
| | (588) | (582) | (583) | (390) | (391) | (590) |
| SCT-Fa132 | GYTFTSYW | IDPSDGYT | ARNYGSNYVGYFDY | QDISNY | YTS | QQGNTLPWT |
| | (399) | (598) | (583) | (584) | (391) | (594) |
| SCT-Fa133 | GYTFTTYW | IDPSDSYS | ARNYGSNYVGYFDY | QDITNY | HTS | QQGNALPWT |
| | (595) | (593) | (583) | (599) | (600) | (590) |
| SCT-Fa134 | GYNFTNYW | IYPSDNKT | AASMDY | QSLLNSKNQKNF | WAS | QNDYSYPYT |
| | (601) | (602) | (603) | (604) | (482) | (605) |
| SCT-Fa135 | GYNFTSYW | IYPSDSKT | ATSMDY | QSLLNSKNQKNF | WAS | QNDYDYPYT |
| | (606) | (607) | (608) | (604) | (482) | (609) |
| SCT-Fa136 | GYTFINYW | IDPSHTST | ARNDYDGGYFDY | QSLVHSNGNTY | TVS | SQTTHVPWT |
| | (610) | (611) | (530) | (348) | (532) | (612) |
| SCT-Fa137 | GYTFSRYW | IDPSDNNT | ARNDYDGGYFDY | QSLVHSNGNTY | KVS | SQSTHVPWT |
| | (613) | (614) | (530) | (348) | (349) | (350) |
| SCT-Fa138 | GYTFTRYW | IDPSSSST | ARNDYDGGYFDY | QSLVHSNGITY | KVS | SQTTHVPWT |
| | (475) | (615) | (530) | (616) | (349) | (612) |
| SCT-Fa139 | GYTFTSYW | IDPSDSST | ARNDYDGGYFDY | QSLLHSNGNTY | KVS | SQTTHVPWT |
| | (399) | (617) | (530) | (618) | (349) | (612) |
| SCT-Fa140 | GYTFTSYW | IDPSSSST | ARNDYDGGYFDY | QSLVHSNGITY | KVS | SQSTHVPWT |
| | (399) | (615) | (530) | (616) | (349) | (350) |
| SCT-Fa141 | GYTFTSYW | IDPSDSNT | SRNDYDGGYFDY | QSLVHSNGNTY | TVS | SQSTHVPWT |
| | (399) | (619) | (620) | (348) | (532) | (350) |
| SCT-Fa142 | GYSFTIYW | IDPSTSNT | ARNDYDGGYFDY | QRLVHSNGNTY | KVS | SQSTHVPWT |
| | (621) | (622) | (530) | (623) | (349) | (350) |
| SCT-Fa143 | GYTFISYW | IDPSDTNT | ARNDYDGGYFDY | QSLVHSNGNTY | KVS | SQTTHVPWT |
| | (568) | (624) | (530) | (348) | (349) | (612) |
| SCT-Fa144 | GYTFTTYW | IDPSDSYR | ARNYGSNYVGYFDY | QDITNY | YTS | QQASTLPWT |
| | (595) | (625) | (583) | (599) | (391) | (626) |
| SCT-Fa145 | GYTFTNYW | IDPSDNYT | AREGAFSSPFAF | QDIRSN | HGT | VQYVQFPRT |
| | (554) | (503) | (627) | (342) | (343) | (344) |
| SCT-Fa146 | GYTFTNYW | IDPSDNYT | AREGAFSSPFAY | QDIRSN | HGT | VQYVQFPRT |
| | (554) | (503) | (504) | (342) | (343) | (344) |
| SCT-Fa147 | GYTFTSYW | IDPSDNYT | AREGAFSSPFAY | QDIRSN | HGT | VQYVQFPRT |
| | (399) | (503) | (504) | (342) | (343) | (344) |
| SCT-Fa148 | GYTFTSYW | IDPSDNYT | AKEGAYSSPFAY | QDIRSN | HGT | VQYVQFPRT |
| | (399) | (503) | (628) | (342) | (343) | (344) |
| SCT-Fa149 | GYTFTTYW | IDPSDNYT | VREGAFSSPFAY | QDIRSN | HGT | VQYVQFPRT |
| | (595) | (503) | (629) | (342) | (343) | (344) |
| SCT-Fa150 | GYSFISHW | IHPSDSDT | AIWSYYYGTRLDY | QNIVHSNGNTY | KVS | QGSHVPYT |
| | (630) | (566) | (631) | (579) | (349) | (580) |
| SCT-Fa151 | GYTFTSYW | IDPSDNNT | ARNDYDGGYFDF | QSLVHSNGHTY | KVS | SQSTHVPWT |
| | (399) | (614) | (632) | (633) | (349) | (350) |
| SCT-Fa152 | GYTFISYC | IDPNNGGP | ARWGWDGFAY | QSLENSNGNTY | RVS | LQVTHVPYT |
| | (634) | (635) | (636) | (637) | (572) | (638) |
| SCT-Fa153 | GYTFISYW | IDPNNGGT | ARWGWDGFAY | QSLENSYGNTY | RVS | LQVTHVPYT |
| | (568) | (639) | (636) | (640) | (572) | (638) |
| SCT-Fa154 | GYTFISYW | IDPNSGGT | TRWGWDGFAY | QSLENSNGNTY | RIS | LQVTHVPYT |
| | (568) | (400) | (641) | (637) | (642) | (638) |
| SCT-Fa155 | GYTFISYW | IDPNSGGT | ARWGWDGFAY | QSLENSNGNTY | RIS | LQVTHVPYT |
| | (568) | (400) | (636) | (637) | (642) | (638) |
| SCT-Fa156 | GYTFISYW | IDPNSGGT | TRWGWDGFAY | QSLENSNGNTY | RIS | LQVTHVPYT |
| | (568) | (400) | (641) | (637) | (642) | (638) |
| SCT-Fa157 | GYTFISYW | IDPNSGIT | ARWGWDGFAY | QSLENSNGNTY | RIS | LQVTHVPYT |
| | (568) | (643) | (636) | (637) | (642) | (638) |
| SCT-Fa158 | GYTFISYW | IDPNSGTT | ARWGWDGFAY | QSLENSNGNTY | RVS | LQVTHVPYT |
| | (568) | (644) | (636) | (637) | (572) | (638) |
| SCT-Fa159 | GYTFISYW | IDPNYGGP | ARWGWDGFAY | QSLENSNGNTY | RVS | LQITHVPYT |
| | (568) | (645) | (636) | (637) | (572) | (646) |
| SCT-Fa160 | GYTFISYW | IDPNYGGP | ARWGWDGFAY | QSLENSNGNTY | RVS | LQLTHVPYT |
| | (568) | (645) | (636) | (637) | (572) | (647) |
| SCT-Fa161 | GYTFISYW | IDPNYGGP | ARWGWDGFAY | SSVSY | LTS | QQWSSNPFT |
| | (568) | (645) | (636) | (648) | (649) | (650) |
| SCT-Fa162 | GYTFISYW | IDPNYGGS | ARWGWDGFAY | QSLENSNGNTY | RVS | LQVTHVPYT |
| | (568) | (651) | (636) | (637) | (572) | (638) |
| SCT-Fa163 | GYTFISYW | IDPNYGGS | ARWGWDGFAY | QDIGSS | ATS | LQYASSPFT |
| | (568) | (651) | (636) | (652) | (653) | (654) |
| SCT-Fa164 | GYTFISYW | INPNNGGT | ARWGWDGFAY | QSLENSNGNTY | RVS | LQVTHVPYT |
| | (568) | (655) | (636) | (637) | (572) | (638) |
| SCT-Fa165 | GYTFVSYW | IDPNNGGS | ARWGWDGFAY | QSLENSNGNTY | RVS | LQLTHVPYT |
| | (656) | (657) | (636) | (637) | (572) | (647) |
| SCT-Fa166 | GYTFVSYW | IDPNNGGS | ARWGWDGFAY | QSLENSNGNTY | RVS | LQLTHVPYT |
| | (656) | (657) | (636) | (637) | (572) | (647) |
| SCT-Fa167 | GYTFISYW | IDPNSGTT | ARWGWDGFAY | QSLENSNGNTY | RVS | LQVTHVPYT |
| | (568) | (644) | (636) | (637) | (572) | (638) |
| SCT-Fa168 | GYTFISYW | IDPNSGTT | ARWGWDGFAY | QSLENSNGNTY | RVS | LQVTHVPYT |
| | (568) | (644) | (636) | (637) | (572) | (638) |
| SCT-Fa169 | GYTFTSYW | IDPSDSYA | ARNYGSNYVGYFDY | QDISNY | YTS | QQEKTLPWT |
| | (399) | (658) | (583) | (584) | (391) | (659) |
| SCT-Fa170 | GYTFRSYG | IYPRSGNT | ARSTAWAGGFDY | QSLFNSRTQKNS | WAS | QNDYTYPWT |
| | (660) | (661) | (662) | (663) | (482) | (664) |
| SCT-Fa171 | GYTFTNYG | IYPRSGNS | ARSTAIVAGGFDY | QSLLNSRIQKNS | WAS | QNDYTYPWT |
| | (665) | (666) | (667) | (668) | (482) | (664) |
| SCT-Fa172 | GYTFTSYG | IYPRSGNT | ARSTYGSSSYAMDY | QSIVHSNGNTY | KVS | FQGSHVPYT |
| | (669) | (661) | (670) | (438) | (349) | (576) |
| SCT-Fa173 | GYTFPDYE | IDPETGGT | TRWAY | SSVSSSY | STS | HHYHRSPPT |
| | (671) | (672) | (673) | (366) | (367) | (674) |
| SCT-Fa174 | GYSITGYY | IYPGSGSI | AREAWGNWYFDV | QSIGTS | YAS | QQSNSWPYT |
| | (675) | (676) | (677) | (678) | (397) | (679) |
| SCT-Fa175 | GYTFSDYE | IDPETGDT | TSNGYPEH | SGVSSTY | STS | HQYHRSPLT |
| | (680) | (681) | (682) | (683) | (367) | (684) |
| SCT-Fa176 | GYTFTDFE | IDPETGGT | TRGIYYGNYGSAMDY | QSIVHSNGNTY | KVS | FQGSHAPLT |
| | (685) | (672) | (686) | (438) | (349) | (687) |
| SCT-Fa177 | GYTFTDYE | IDPETGGT | TRWAF | QSLVHSNGNTY | KVS | SQSTHVPYT |
| | (688) | (672) | (689) | (348) | (349) | (690) |
| SCT-Fa178 | GYTFTDYE | IDPETGGT | TRWAF | SSVSSSY | STS | HHYNRSPPT |
| | (688) | (672) | (689) | (366) | (367) | (691) |
| SCT-Fa179 | GYAFSNYL | INPGNGGT | ARTAYGSPLDY | QDINRY | RAN | LQYDEFPYT |
| | (692) | (693) | (694) | (695) | (459) | (463) |
| SCT-Fa180 | GYAFTNYL | INPGSGAT | | QDINRY | REN | LQYDEFPYT |
| | (696) | (697) | (698) | (695) | (699) | (463) |
| SCT-Fa181 | GYAFTNYL | INPGSGAT | ARRTTVVAPYAMDY | QDISNY | YTS | QQGNTLPRT |
| | (696) | (697) | (700) | (584) | (391) | (701) |
| SCT-Fa182 | GYAFTNYL | ISPGSGAT | ARSAYGSPLDF | QDINRY | RVN | QQYDEFPYT |
| | (696) | (702) | (703) | (695) | (704) | (705) |
| SCT-Fa183 | GYAFTNYL | ISPGSGAT | ARSAYGSPLDF | QNIGTS | YAS | QQSNNWPFT |
| | (696) | (702) | (703) | (706) | (397) | (707) |
| SCT-Fa184 | GYAFTNYL | ISPGSGGT | ARSAYGHVFDY | QDINSY | RAN | LQYDEFPYT |
| | (696) | (708) | (709) | (455) | (459) | (463) |
| SCT-Fa185 | GYAFTNYL | ISPGSGGT | ARSAYGHVFDY | QDINSY | RAN | LQYDEFPYT |
| | (696) | (708) | (709) | (455) | (459) | (463) |
| SCT-Fa186 | GYTFRSNG | IYPRSGNI | ARSTTWAGGFDF | QSLLNSRTQKNS | WAS | QNDYTYPWT |
| | (710) | (711) | (712) | (713) | (482) | (664) |
| SCT-Fa187 | GYSFTGYY | IYPGSGNT | AREAWGNWYFDV | QSIGTS | YAS | QQSNSWPYT |
| | (714) | (511) | (677) | (678) | (397) | (679) |
| SCT-Fa188 | GYSFTGYY | IYPGSGNT | AREAWGNWYFDV | QSIGTS | YAS | QQSNSWPYT |
| | (714) | (511) | (677) | (678) | (397) | (679) |
| SCT-Fa189 | GYSFTGYYI | IGWIYPGS | AREAWGNWYFDV | QSIGTS | YAS | QQSNSWPYT |
| | (715) | (716) | (677) | (678) | (397) | (679) |
| SCT-Fa190 | GYTFNTYW | IDPSDSYT | ARNYGSNYVGYFDY | QDISNY | YTS | QQAKALPWT |
| | (586) | (582) | (583) | (584) | (391) | (587) |
| SCT-Fa191 | GYTFTSFG | IYPRSGNI | ARSTAWAGGFDY | QSLLNSRIQKNS | WSS | QNDYNYPWT |
| | (717) | (711) | (662) | (668) | (718) | (719) |
| SCT-Fa192 | GYTFTNYG | IYPRSGNI | ARSTAWAGGFDY | QSLLNSRIQKNS | WAS | QNDYNYPWT |
| | (665) | (711) | (662) | (668) | (482) | (719) |
| SCT-Fa193 | GYTFTSYG | IYPRSGNI | ARSTAWAGGFDY | QSLLNSRIQKNS | WAS | QNDYNYPWT |
| | (669) | (711) | (662) | (668) | (482) | (719) |
| SCT-Fa194 | GYTLTDYY | IYPGNGNT | ARGGSSDRIYYYAMDY | QNINVW | KAS | QQGQSYPLT |
| | (441) | (525) | (526) | (354) | (355) | (527) |
| SCT-Fa195 | GYTLTDYY | IYPGNGNT | ARGGSSDRIYYYAMDY | DHINNW | GAI | QQYWNTPYT |
| | (441) | (525) | (526) | (720) | (721) | (722) |

TABLE 3

| mAb AA ID | VH Amino Acid Sequence | SEQ ID NO: | VL Amino Acid Sequence | SEQ ID NO: |
|---|---|---|---|---|
| SCT-Fa001 | EFQLQQSGPELVKPGASVKISCKASGNSFTDYN LNWVKQSHGKSFEWIGVINPNYGTTSYNQKFKV KAALTVDQSSSTAYMQLNSLTSEDSAVYYCARE GNGWGQGTLVTVSA | 4 | DVVMTQTPLTLSVTIGQPVSISCKSSQSLLDSD GKTYLNWLLQRPGQSPQRLIYLVSKLDSGVPDR FTGSGSGTDFTLKISRVEAEDLGVYYCWQGTLF PWTFGGGTELEIK | 317 |
| SCT-Fa002 | EFQLQQSGPELVKPGASVKISCKASGYSFTDYN MNWVKQSNGKSLEWIGVNNPKYGTTTYNQKFKG KATLTVDQSSSTAYMQLNSLTSEDSAVYYCARR GYYYGSSYGYFDVWGTGTTVTVSS | 5 | DILMTQSPSSMSVSLGDRVSIICHASQDIRSNI GWLQQKPGKSLKGLIYHGTNLEDGVPSRFSGSG SGADYSLTISSLESEDFADYYCVQYVQFPRTFG GGTKLEIK | 211 |
| SCT-Fa003 | EGKLEESGGGLVQPGGSMKLSCAASGFTFSDAW MDWVRQSPKKGLEWVAEIRNKANKHSKHYAESV KGRFTISRDDSKSSVYLQMNSLRAEDTGIYYCS YDYIFAYWGQGTLVTVSA | 6 | DVVMTQTPLSLPVSLGDQASISCRSSQSLVHSN GNTYLHWYLQKPGQSPKLLIYKVSNRFSGVPDR FSGSGSGTDFTLKISRVEAEDLGVYFCSQSTHV PWTFGGGTKLEIK | 302 |
| SCT-Fa004 | EVHLQQSGAEFMKPGASVKLSCTASGFNIKGHY MHWVKQRTEQGLEWIGRIDPDDGATKFAPKFQG KATITADTSSNTVYLQLSSLTSEDTAVYYCTRS NPYYFDYWGQGTTLTVSS | 7 | DIQMNQSPSSLSASLGDTISITCHASQNINVWL SWYQQKPGNIPKLLIYKASILHTGVPSRFSGSG SVTGFTLTISSLQPEDIASYYCQQGQNYPYTFG GGTKLEIK | 223 |
| SCT-Fa005 | EVHLQQSVAEVVRPGASVNLSCTVSGFNNKNTY MYWVKQRPEQGLEWIGIIDPANGNVKYAPKFQG KATITADTSSNTAYLQLSSLTSEDTAIYYCARG SSRLYYALDYWGQGTSVTVSS | 8 | DIVLTQSPASLAVSLGQRATISCRASESVDSYG NSFMHWYQQKPGQPPKLLIYRASNLESGIPARF SGSGSRTDFTLTIYPVEADDVATYYCQQSNEDP YTFGGGTKLEIK | 260 |
| SCT-Fa006 | EVHLVESGGGLVKPGGSLKLSCAASGFTFSDYG MHWVRQAPEKGLDWIAYISSGSISSDSSTIYYA DTVKGRFTISRDNAKNTLFLQMTSLRSEDTAMY YCARHDWRGLYGMDYWGQGTSVTVSS | 9 | QIVLTQSPAIMSASPGKKVTLTCSASSSVSSSY LYWYQQKPGSSPKLWIYSTSNLASGVPARFSGS GSGTSYSLTISSMEAEDAASYFCHQWSSYPPTF GGGTKLEIK | 325 |
| SCT-Fa007 | EVKLVESEGGLVHPGSSMKLSCTASGFTFSDYY MAWVRQVPEKGLEWIANINFDGSGTYYLDSLKS RFIISRDNGKNILYLQMSGLKSEDTATYYCARL YDGYYAYWGQGTTLTVSS | 10 | DVVMTQTPLSLPVSLGDQASISCRSSQSLVHSN GNTYLYWYLQKPGQSPKLLIYKVSNRFSGVPDR FSGSGSGTDFTLKISRVEAEDLGVYFCSQSTHV PLTFGAGTKLELK | 305 |
| SCT-Fa008 | EVKLVESEGGLVQLGSSMKLSCTASGFTFSDYY MAWVRQVPEKGLEWIANINFDGSGTYYLDSLKS RFIISRDNGKNILYLQMSGLKSEDTATYYCARL YDGYYAYWGQGTTLTVSS | 11 | DVVVTQTPLSLPVSLGDQASISCRSSQSLVHSN GNTYLYWYLQKPGQSPKLLIYKVSNRFSGVPDR FSGSGSGTDFTLKISRVEAEDLGVYFCSQSTHV PLTFGAGTKLELK | 319 |
| SCT-Fa009 | EVKLVESEGGLVQPGSSMKISCTASGFTFSDYY MAWVRQVPEKGLEWVANINYDGSRTYYLDSLKS RFIISRDNAKNILYLQMSSLKSEDTATYYCARN IYYYGGSSYGMDYWGQGTSVTVSS | 12 | DIQMTQSPASLSASVGETVTITCRASGNIHNYL AWYQQKQGKSPQLLVYNAETLADGVPSRFSGSG SGTQYSLKINSLQPEDFGTYYCQHFWSTPLTFG AGTKLELK | 241 |
| SCT-Fa010 | EVKLVESEGGLVQPGSSMKLSCTASGFTFSDYY MAWVRQVPEKGLEWIANINFDGSGTYYLDSLKS RFIISRDNAKNILYLQMSSLKSEDTATYYCARL YDGSLDYWGQGTTLTVSS | 13 | DVVMTQTPLSLSVTIGQPASISCKSSQSLLYTN GKTYLSWLLQRPGQSPKRLIYLVSKLDSGVPDR FTGSGSGTDFTLKISRVEAEDLGAYYCVQGTHF PLTFGGGTKLEIK | 306 |
| SCT-Fa011 | EVKLVESEGGLVQPGSSMKLSCTASGFTFSDYY MAWVRQVPEKGLEWVAHINFDGSGTYYLDSLKS RFIISRDNAKNILYLQLSSLKSEDTATYYCARL YDGSHDYWGQGTTLTVSS | 14 | DVVMTQTPLTLSVTIGQPASISCKSSQSLLYSN GKTYLSWLFQRPGQSPKRLIYLVSTLDSGVPDR FTGSGSGTDFTLKISRVEAEDLGVYYCVQGTHF PLTFGGGTKLEIK | 315 |
| SCT-Fa012 | EVKLVESEGGLVQPGSSMKLSCTASGFTFSDYY MAWVRQVPEKGLEWVAHINFDGSGTYYLDSLKS RFIISRDNAKNILYLQLSSLKSEDTATYYCARL YDGSHDYWGQGTTLTVSS | 14 | DVVMTQTPLTLSVTIGQPASISCKSSQSLLYSN GKTYLSWLLQRPGQSPKRLIYLVSTLDSGVPDR FTGSGSGTDFTLKISRVEAEDLGVYYCVQGTHF PLTFGGGTKLEIK | 316 |
| SCT-Fa013 | EVKLVESEGGLVQPGSSMKLSCTASGFTFSDYY MAWVRQVPEKGLEWVAHINFDGSGTYYLDSLKS RFIISRDNAKNILYLQLSSLKSEDTGTYYCARL YDGSHDYWGQGTTLTVSS | 15 | DVVMTQTPLTLSVTIGQPASISCKSSQSLLYSN GKTYLGWLFQRPGQSPKRLIYLVSTLDSGVPDR FTGSGSGTDFTLKISRVEAEDLGVYYCVQGTHF PLTFGGGTKLEIK | 312 |
| SCT-Fa014 | EVKLVESEGGLVQPGSSMKLSCTASGFTFSDYY MAWVRQVPEKGLEWVAHINFDGSGTYYLDSLKS RFIISRDNAKNILYLQLSSLKSEDTGTYYCARL YDGSHDYWGQGTTLTVSS | 15 | DVVMTQTPLTLSVTIGQPASISCKSSQSLLYSN GKTYLNWLLQRPGQSPKRLIYLVSTLDSGVPDR FTGSGSGTDFTLKISRVEAEDLGVYYCVQGTHF PLTFGGGTKLEIK | 314 |
| SCT-Fa015 | EVKLVESEGGLVQPGSSMKLSCTASGFTFSDYY MAWVRQVPEKGLEWVAHINFDGSGTYYLDSLKS RFIISRDNEKSILYLQLSSLKSEDTATYYCARL YDGSHDYWGQGTTLTVSS | 16 | DVVMTQTPLTLSVTIGQPASISCKSSQSLLYSN GKTYLNWLLQRPGQSPKRLIYLVSTLDSGVPDR FTGSGSGTDFTLKISRVEAEDLGVYYCVQGTHF PLTFGGGTKLEIK | 314 |
| SCT-Fa016 | EVKLVESEGGLVQPGSSMKLSCTASGFTFSDYY MAWVRQVPEKGLEWVAHINFDGSGTYYLDSLKS RFIISRDNTKNILYLQLSSLKSEDTATYYCARL YDGSHDYWGQGTTLTVSS | 17 | DWLTQTPLTLSVTIGQPASISCKSSQSLLYSN GKTYLNWLLQRPGQSPKRLIYLVSTLDSGVPDR FTGSGSGTDFTLKISRVEAEDLGVYYCVQGTHF PLTFGGGTKLEIK | 288 |
| SCT-Fa017 | EVKLVESEGGLVQPGSSMKLSCTASGFTFSDYY MAWVRQVPEKGLEWVANINFDGSGTYYLDSLKS RFIISRDNAKNILYLQMSSLKSEDTATYYCARL YDGSHDYWGQGTTLTVSS | 18 | DVVMTQTPLTLSVTIGQPASISCKSSQSLLYSN GKTYLNWLLQRPGQSPKRLIYLVSKLDSGVPDR FTGSGSGTDFTLKISRVEAEDLGVYYCVQGTHF PQTFGGGTKLEIK | 313 |
| SCT-Fa018 | EVKLVESEGGLVQPGSSMKLSCTASGFTFSDYY MAWVRQVPEKGLEWVANINFDGSSSYYLDSLKS RFIISRDNTKNILYLQMSSLKSEDTATYYCARL YDGYYAYWGQGTTLTVSS | 19 | DVVMTQTPLSLPVSLGDQASISCRSSQSLIHSN GNTYLYWYLQKPGQSPTLLIYKVSNRFSGVPDR FSGSGSGTDFTLKISRVEAEDLGVYFCSQSTHV PLTFGAGSKLELK | 298 |
| SCT-Fa019 | EVKLVESEGGLVQPGSSMKLSCTASGFTFSDYY MSWVRQVPEKGLEWVANINFDGSGTYYLDSLKS RFIISRDNAKNILYLQMSSLRSEDTATYYCARL YDGSHDYWGQGSALTVSS | 20 | DVVMTQTPLTLSITIGQPAAVSCKSSQSLLYSN GKTYLNWLLQRPGQSPKRLIYLVSKLDSGVPDR FTGSGSGTDFTLKISRVEAEDLGVYYCVQATHF PLTFGGGTKLEIK | 307 |
| SCT-Fa020 | EVQLHQSGPELVKPGASVKMSCKASGYTFTDYN MHWVKQSHGKSLEWIGYINPNNGGIIYNQNFKG KATLTVNKSSTTAYMELRSLTSEDSAVYYCVGS LDYWGQGTTLTVSS | 21 | DIQMTQTTSSLSASLGDRVTISCRASQDINNYL NWYQQKPDETVKLLIYYTSRLHSGVPSRFSGSG SGTDYSLTISNLDQEDFATYFCQQGYMLPLTFD AGTKLELK | 246 |
| SCT-Fa021 | EVQLHQSGPVLVKPGASVKMSCKASEYTFADYY MNWVKQSHGKSLEWIGVINPYNGGATYNQKFKG KATLTVDKSSSTAYMEFSSLTSEDSAVYYCARD ADFYGNSYVGYAMDYWGQGTSVTVSS | 22 | DIVLTQSPATLSVTPGDSVSLSCRASQSINTDL HWYQQKSHESPRLLIKYASQSISGIPSRFSGSG SGTDFSLSINSVETEDFGIYFCQQSHTWPQYTF GGGTKLEIK | 261 |
| SCT-Fa022 | EVQLQQPGAELVKPGASVKLSCKASGYTFTSYW IHWVKQRPGRGLEWIGRIDPNSGGTKYNEKFKS KATLTVDKPSSTAYMQLSSLTSEDSAVYYCTKD GNTPYYAMDYWGQGTSVTVSS | 23 | SIVMTQTPKFLLVSAGDRVTITCKASQSVSNAV AWYQQKPGQSPKLLIYYASNRYTGVPDRFTGSG YGTDFTFTISTVQAEDLAVYFCQQDYSSPLTFG AGTKLELK | 330 |
| SCT-Fa023 | EVQLQQSGAELVKPGASVKLSCTASDFNIKDYY LHWVKQRTEQGLEWIGRIDPDDGETKYGPKFQG KATITADTSSNTAFLQLSSLTSEDTAVYFCSRS NPYYFDYWGQGTTLTVSS | 24 | DIQMNQSPSSLSASLGDTITITCHVSQNINVWL SWYQQKPGNIPKLLIYKASNLHTGVTSRFSGSG AGTGFTLTISSLQPEDIATYYCQQGQNYPYTFG GGTKLEIK | 237 |
| SCT-Fa024 | EVQLQQSGAELVKPGASVKLSCTASDFNIKDYY MHWVKQSTEQGLEWIGRIDPEDDETKYAPKFQG KATITADTSSNTAYLQLSSLTSEDTAVYYCARS NPYYFDYWGQGTSLTVSS | 25 | DIQMNQSPSSLSASLGDTITITCHVSQNINVWL SWYQQKPGNIPKLLIYKASNLHTGVTSRFSGSG AGTGFTLTISSLQPEDLATYYCQQGQNYPYTFG GGTKLEIK | 238 |
| SCT-Fa025 | EVQLQQSGAELVKPGASVKLSCTASGFNIKDFY MHWVHQRTEKGLEWIGRIDPEDGKTKFAPKFQG KATITADTSSNTAYLQLSSLTSEDTAVYYCARS NPYFFDYWGQGTTLTVSS | 26 | DIQMNQSPSSLSASLGDTITITCHASQTINVWL SWYQQKPGNIPKLLIYKASNLHTGVPSRFSGSG SGTGFTLNISSLQPEDIATYYCHQGQSYPYTFG GGTKLEIK | 236 |
| SCT-Fa026 | EVQLQQSGAELVKPGASVKLSCTASGFNIKDFY MHWVHQRTEKGLEWIGRIDPEDGKTKYAPKFQG KATITADTSSNTAYLQLSSLTSEDTAVYYCARS NPYFFDYWGQGTTLTVSS | 27 | DIQMNQSPSSLSASLGDTITITCHASQTINVWL SWYQQKPGNIPKLLIYKASNLHTGVPSRFSGSG SGTGFTLNISSLQPEDIATYYCHQGQSYPYTFG GGTKLEIK | 236 |
| SCT-Fa027 | EVQLQQSGAELVKPGASVKLSCTASGFNIKDYY IHWVKQRTDQGLEWIGRIDPDNGETKYAPKFQG KATITADTSSNTAYLQLGSLTSEDTAVYYCARS SPYYFDYWGQGTTLTVSS | 28 | DIQMNQSPSSLSASLGDTITITCHASQNINVWL SWYQQKPGNIPKLLIYKASNLHTGVPSRFSGSG SVTGFTLTISSLQPEDIATYYCQQGQNFPYTFG GGTKLEII | 231 |
| SCT-Fa028 | EVQLQQSGAELVKPGASVKLSCTASGFNIKDYY MHWVKQRTDHGLEWIGRIDPDNGETKYAPKFQG KATITADTSSNTAYLQLGSLTSEDTAVYYCARS SPYYFDYWGQGTTLTVSS | 29 | DIQMNQSPSSLSASLGDTITITCHASQNINVWL SWYQQKPGNIPKLLIYKASNLHTGVPSRFSGSG SVTGFTLTISSLQPEDIATYYCQQGQNFPYTFG GGTKLEIK | 232 |
| SCT-Fa029 | EVQLQQSGAELVKPGASVKLSCTASGFNIKDYY MHWVKQRTDQGLEWIGRIDPEDGKTKYAPKFQG KATITADTSSNTAFLQLTSLTSEDTAVYYCARS NPYYFDYWGQGTTLTVSS | 30 | DIQMNQSPSSLSASLGDTITITCHASQNINVWL GWYQQKPGNIPKLLIYKASNLHTGVPSRFSGSG SVTSFTLTISSLQPEDIATYYCQQGQSYPYTFG GGTKLEIK | 224 |
| SCT-Fa030 | EVQLQQSGAELVKPGASVKLSCTASGFNIKDYY MHWVKQRTEQGLEWIGRIDPEDGETKYAPKFQG KATITADTSSNTASLQLSSLTSEDTAVYYCARS NPYFFDYWGQGTPLTVSS | 31 | DIQMNQSPSSLSASLGDTITITCHASQTINVWL SWYQQKPGNIPKLLIYKASKMHTGVPSRFSGSG SGTGFTLNISSLQPEDIATYYCHQGQSYPYTFG GGTKLEIK | 235 |
| SCT-Fa031 | EVQLQQSGAELVKPGASVKLSCTASGFNIKDYY MHWVKQRTEQGLEWIGRIDPEDGETKYVPKFQG KATITADTSSNTAFLHLTSLTSEDTAVYYCARS NPYYFDYWGQGTTLTVSS | 32 | DIQMNQSPSSLSASLGDTVTITCHASQTINVWL SWYQQKPGNIPKLLIYKASNLHTGVPSRFSGSG SGTGFTLTISSLQPEDIATYYCQQGQNYPYTFG GGTKLEIK | 240 |
| SCT-Fa032 | EVQLQQSGAELVKPGASVKLSCTASGFNIKDYY MHWVRQRTEQGLEWIGRIDPEDGETKYAPKFQG KATITAATSSNTPYLQLSGLTSEDTAVYYCARS NPYYFDYWGQGTTLTVSS | 33 | DIQMNQSPSSLSASLGDRITINCHASQNINVWL SWYQQKPGNIPKLLIYKASNLHTGVPLRFSGSG SVTVFTLTISSLQPEDIATYYCQQGQNYPYTFG GGTKLEIK | 220 |
| SCT-Fa033 | EVQLQQSGAELVKPGASVKLSCTASGFNIKDYY MHWVRQRTEQGLEWIGRIDPEDGETKYAPKFQG KATITAATSSNTPYLQLSGLTSEDTAVYYCGRS NPYYFDYWGQGTTLTVSS | 34 | DIQMNQSPSSLSASLGDRITINCHASQNINVWL SWYQQKPGNIPKLLIYKASNLHTGVPSRFSGSG SVTVFTLTISSLQPEDIATYYCQQGQNYPYTFG GGTKLEIK | 221 |
| SCT-Fa034 | EVQLQQSGAELVKPGASVKLSCTDFGFNIKDYY MHWVKQRTEQGLEWIGRIDPEDGETKYVPKFQG KATITADTSSNTAFLHLTSLTSEDTAVYYCARS NPYYFDYWGQGTTLTVSS | 35 | DIQMNQSPSSLSASLGDTVTITCHASQTINVWL SWYQQKPGNIPKLLIYKASNLHTGVPSRFSGSG SGTGFTLTISSLQPEDIATYYCQQGQNYPYTFG GGTKLEIK | 240 |
| SCT-Fa035 | EVQLQQSGAELVKPGASVKLSCTDFGFNIKDYY MHWVKQRTEQGLEWIGRIDPEDGETKYVPKFQG KATITADTSSNTAFLHLTSLTSEDTAVYYCARS NPYYFDYWGQGTTLTVSS | 35 | DVVMTQTPLTLSVTIGQPVSISCKSSQSLLDSD GKTYLNWLLQRPGQSPQRLIYLVSKLDSGVPDR FTGSGSGTDFTLKISRVEAEDLGVYYCWQGTLF PWTFGGGTELEIK | 317 |
| SCT-Fa036 | EVQLQQSGAELVKPGASVKLSCTPSDFNIKDYY IHWVRQRTEQGLEWIGRIDPEDGETKYAPKFQG KATITADTSSNTAYLQVSSLTSEDTAVYYCARS NPYYFDYWGQGTTLTVSS | 36 | DIVMTQSQKFMSTSVGDRVSVTCKASQNVGTNV AWYQQKPGQSPKALIYSASYRYSGVPDRFTGSG SGTDFTLTISNVQSEDLAEYFCQQYNTYPLTFG AGTKLELK | 277 |
| SCT-Fa037 | EVQLQQSGAELVKPGASVKLSCTVSGFNIKDYY MHWVKQRTEQGLEWIGRIDPDNDKIKYAPKFQG RATITADTSSNTVYLQLSSLTSEDTAVYYCARS NPYFFDYWGQGTTLTVSS | 37 | DIQMNQSPSSLSASLGDTITITCRASQTINIWL SWYQQKPGNIPKLLIYKASTLHTGVPSRFSGSG SGTGFTLNISSLQPEDIATYYCHQGQSYPYTFG GGTKLEIK | 239 |
| SCT-Fa038 | EVQLQQSGAELVKPGASVKLSCTVSGFNIKDYY MHWVKQRTEQGLEWIGRIDPENDKIKYAPKFQG KATITADTSSNTVYLQLSSLTSEDTAVYYCARS NPYFFDYWGQGTTLTVSS | 38 | DIQMNQSPSSLSASLGDTITITCHASQTINVWL SWYQQKPGNIPKLLIYKASNLHTGVPSRFSGSG SGTGFTLNISSLQPEDIATYYCHQGQSYPYTFG GGTKLEIK | 236 |
| SCT-Fa039 | EVQLQQSGAELVKPGASVKLSFTASDFNIKDYY IHWVKQRTEQGLEWIGRIDPEDGETRYAPKFQG KATITADTSSNTAYLQLSSVTSADTAVYFCTRS NPYYFDYWDQGTTLTVSS | 39 | DIQMNQSPSSLSASLGDTITITCHASQNINVWL SWYQQKPGNIPKLLIYKASNLHTGVPSRFSGSG SGTGFTLTISSLQPEDIATYYCQQGQNFPYTFG GGAKLEIK | 228 |
| SCT-Fa040 | EVQLQQSGAELVKPGASVKLSFTASGFNIKDYY IHWVKQRTEQGLEWIGRIDPEDGETKYAPKFQG KATITADTSSNTAYLQLSSLTSEDTAVYFCTRS NPYYFDYWGQGTTLTVSS | 40 | DIQMNQSPSSLSASLGDTITITCHASQNINVWL SWYQQKPGNIPKLLIYKASNLHTGVPSRFSGSG SGTGFTLTISSLQPEDIATYYCQQGQNFPYTFG GGAKLEIK | 228 |
| SCT-Fa041 | EVQLQQSGAELVKPGASVKLSYTSSGFNIKDYY IHWVKQRTDQGLEWIGRIDPDNGETKYAPKFQG KATIIADTSSNTAYLQLGSLTSEDTAVYYCARS SPYYFDYWGQGTTLTVSS | 41 | DIQMNQSPSSLSASLGDTITITCHASQNINVWL SWYQQKPGNIPKLLIYKASNLHTGVPSRFSGSG SVTGFTLTISSLQPEDIATYYCQQGQNFPYTFG GGTKLEII | 231 |
| SCT-Fa042 | EVQLQQSGAELVKPGASVKLSYTSSGFNIKDYY IHWVKQRTDQGLEWIGRIDPDNGETKYAPKFQG KATITADTSSNTAYLQLGSLTSEDTAVYYCARS SPYYFDYWGQGTTLTVSS | 42 | DIQMNQSPSSLSASLGDTITITCHASQNINVWL SWYQQKPGNIPKLLIYKASNLHTGVPSRFSGSG SVTGFTLTISSLQPEDIATYYCQQGQNFPYTFG GGTKLEIK | 232 |
| SCT-Fa043 | EVQLQQSGAELVRPGASVKLSCTASGFNIKDYY MHWVKQRTEQGLEWIGRIDPDNDEIKYAPKFQG KATLTADTSSNTASLQLSGLTSEDTAVYYCARS SPYYFDYWGQGTTLTVSS | 43 | DIQMNQSPSSLSASLGDTITITCHASQNINVWL SWYQQKPGNIPKLLIYKASNLHTGVPSRFSGSG SVTGFTLTISSLQPEDIATYYCQQGQNIPYTFG GGTQLEIK | 233 |
| SCT-Fa044 | EVQLQQSGAELVRPGASVKLSCTASGFNIKDYY MHWVKQRTEQGLEWIGRIDPDNDEIKYAPKFQG KATLTSDTSSNTASLQLSGLTSEDTAVYYCARS SPYYFDYWGQGTTLTVSS | 44 | DIQMNQSPSSLSASLGDTITITCHASQNINVWL SWYQQKPGNIPKLLIYKASNLHTGVPSRFSGSG SVTGFTLTISSLQPEDIATYYCQQGQNIPYTFG GGTQLEIK | 233 |
| SCT-Fa045 | EVQLQQSGAEVVRPGASVKLSCTSSGFNIKDYY LHWVMQRTEHGLNWIGRIDPDDAETKYAPQFQG RATITADTSSNTAYLQLSSLTSEDTAVYYCTRD FGYTDYFDYWGQGTTLTVSS | 45 | DIVMTQSHKFMSTSVRDRVSITCKASQDVSTAV AWYQQKPGQSPKLLIYSASYRYTGVPDRFTGSG SGTDFTFTISSVQAEDLAVYYCQQHYSTPTFGG GTKLEIQ | 269 |
| SCT-Fa046 | EVQLQQSGPELVKPGASVKISCKASGYTFTDYN MHWVKQSHGKSLAWIGYIYPYNGGTGYNQKFKN KATLTVDDSSSTAYMDLRSLTSDDSAVYYCARE DYGSREWFAYWGQGTLVTVSA | 46 | DVVMTQTPLSLPVSLGDQASISCRSSQSLVHSN GNTYLYWYLQKPGQSPKLLIYKVSNRFSGVPDR FSGSGSGTDFTLKISRVEAEDLGVYFCSQSTHV PLTFGAGTKLELK | 305 |
| SCT-Fa047 | EVQLQQSGPELVKPGASVKISCKASGYTFTDYY MNWVKQSHGKSLEWIGDINLNNGGTVYNQKFRG KATLTVDKSSSTAYMELRSLTSEDSAVYYCEGL GGYWGQGTTLTVSS | 47 | DVLMTQTPLSLPVSLGDQVSISCRSSQSIVHSN GNTYLEWYLQKPGQSPNLLIYKVANRLSGVPDR FSGSGSGTDFTLKISRVEAEDLGVYYCFQGSHV PFTFGSGTKLEIK | 284 |
| SCT-Fa048 | EVQLQQSGPELVKPGASVKISCKASGYTLTDYY MNWVKQSHGESLEWIGDINPNNGDINYNQKFKG KATLTADKSSSTAYMELRSLTSEDSAVYYCEGL GGYWGQGTTLTVSS | 48 | DVLMTQTPLSLPVSLGDQASISCRSSQTIVHSN GNTYLEWYLQTPGQSPKLLIYKVSNRISGVPDR FSGSGSGTDFTLKISRVEAEDLGVYYCFQGSHV PFTFGSGTKLEIK | 283 |
| SCT-Fa049 | EVQLQQSGPELVKPGSSVKISCKASGYTLTDYY MNWVKQSHGESLEWIGDINPNNGDINYNQKFKG KATLTADKSSSTAYMELRSLTSEDSAVYYCEGL GGYWGQGTTLTVSS | 49 | DVLMTQTPLSLPVSLGDQASISCRSSQTIVHSN GNTYLEWYLQTPGQSPKLLIYKVSNRISGVPDR FSGSGSGTDFTLKISRVEAEDLGVYYCFQGSHV PFTFGSGTKLEIK | 283 |
| SCT-Fa050 | EVQLQQSGPMLVKPGASVKMSCKASGNTFTAFY MSWVKQSHGKSLEWIGVVNPYNGGAGYNQKFKG KATLTVDKSSSTAYMELNGLTSEDSAVYYCLLW EFAHWGQGTLVTVSA | 50 | DVVMTQTPLTLSVTLGQPASISCKSSQSLLDSD GKTYLNWLLQRPGQSPKRLIYLVSKLDSGVPDR FTGSGSGTDFTLKISRVEAEDLGIYYCWQGTHS WTFGGGTKLEIK | 318 |
| SCT-Fa051 | EVQLQQSGPVLVKPGASVKMSCKASEYTFTDYY MNWVKQSHGKTLEWIGVINPYNGGATYNQRFKD KATLTVDKSSSTAYMELNSLTSEDSAVYYCARD VDFYGNRYVGYAMDYWGQGTSVTVSS | 51 | DIVLTQSPATLSVTPGDSVSLSCRASQSISNNL HWYQQKSHESPRLLIKYASQSISGIPSRFSGSG SGTDFTLSINSVETEDFGIYFCQQSNSWPQYTF GGGTKLEIK | 262 |
| SCT-Fa052 | EVQLQQSGPVLVKPGASVKMSCKASGYSFTGYH MNWVKQSHGKSLEWIGIINPYNSASTYNQKFKG KATLTVDKSSSTAYMELNSLTSEDSWYYCVRG AYGNPLDYWGQGTTLTVSS | 52 | DIKMTQSPSSMYASLGERVTITCKASQDINSYL SWFQQKPGKSPKTLIYRTNRLVDGVPSRFSGSG SGQDYSLTISSLEYEDMGIYYCLQYDEYPYTFG GGTKLEIK | 206 |
| SCT-Fa053 | EVQLQQSGPVLVKPGASVKMSCKASGYSFTGYH MNWVKQSHGKSLEWIGIINPYNSATTYNQKFKG KATLTVDKSSSTAYMELNSLTSEDSWYYCVRG AYGNPLDYWGQGTTLTVSS | 53 | DIKMTQSPSSMYASLGERVTITCKASQDINSYL SWFQQKPGKSPKTLIYRANRLVDGVPSRFSGSG SGQDYSLTISSLEYEDMGIYYCLQYDEYPYTFG GGTKLEIK | 205 |
| SCT-Fa054 | EVQLQQSGPVLVKPGASVKMSCKASGYSFTGYH MNWVKQSHGKSLEWIGIINPYNSATTYNQKFKG KATLTVDKSSSTAYMEVNSLTSEDSLVYYCVRG AYGNPLDYWGQGTTLTVSS | 54 | DIKMTQSPSSMYASLGERVTITCKASQDINSYL SWFQQKPGKSPKTLIYRANRLLDGVPSRFSGSG SGQDYSLTISSLEYEDMGIYYCLQYDEYPYTFG GGTKLEIK | 204 |
| SCT-Fa055 | EVQLQQSGPVLVKPGASVKMSCKASGYTFTDYH MNWVKQSHGKSLEWIGVISPYNGGTSQNQKFKG KATLTVDKSSSTAYMELNRLTSEDSAVYYCARS DYGSPLDYWGQGTTLTVSS | 55 | DIKMTQSPSSMYASLGERVTITCKASQDINSYL RWFQQKPGKSPKTLIYRANRLVDGVPSRFSGSG SGQDYSLNISSLEYEDMGIYYCLQYDEFPYTFG GGTKLEIK | 202 |
| SCT-Fa056 | EVQLQQSGPVLVKPGASVKMSCKASGYTFTDYY MNWVKQSHGKSLEWIGVINPYNGGATYNQNFKG KATLSVDKSSSTAYMELNSLTSEDSAVYYCARD VDFYDNAYVGYAMDYWGQGTSVTVSS | 56 | DIVLTQSPATLSVTPGDSVSLSCRASQTISNNL HWYQQKSHESPRLLIKYASQSISGIPSRFSGSG SGTDFTLRINSVETEDFGMYFCQQSNSWPQYTF GGGTKLEIK | 263 |
| SCT-Fa057 | EVQLQQSGPVLVKPGASVKMSCKASGYTFTDYY MNWVKQSHGKSLEWIGVINPYNGGTSYNQKFKG KATLTVDKSSSTAYMELNSLTSEDSAVYYCARG GDYDVNYSAMDYWGQGTSVTVSS | 57 | DVVMTQTPLTLSVTIGQPASISCKSSQSLLDSD GKTYLNWLLQRPGQSPKRLIFLVSKLDSGVPDR FTGSGSGTDFTLKISRVEAEDLGVFYCWQGTHF PTWTFGGGTKLEIK | 310 |
| SCT-Fa058 | EVQLQQSGPVLVKPGASVKMSCKVSEYTFTDYY MNWVKQSHGKSLEWIGVINPYNGGVTYNQKFKG KATLTVDKSSSTAYMELSSLTSEDSAVYYCARD ADFHGNSYVGYAMDYWGQGTSVTVSS | 58 | EIVLTQSPATLSVTPGDSVSLSCRASQSISNDL HWYQQKSHDSPRLLIKYASQSISGIPSRFSGSG SGTDFTLSINSVETEDFGIYFCQQSHSWPQYTF GGGTKLEIK | 320 |
| SCT-Fa059 | EVQLQQSGPVLVKPGASVKMSCQASGYSFTGYH MNWVKQSHGKSLEWIGIINPYNSGITYNQKFKG RATLTVDKSSDTAYMDLNSLTSEDSWYYCVRG AYGNPLDYWGQGTTLTVSS | 59 | DIRMTQSPSSIYASLGERVTITCKASQDINSYL SWFQQKPGKSPETLIYRTNRLVDGVPSRFSGSG SGQDYSLTISSLEYEDMGIYYCLQYDEYPYTFG GGTKLEIK | 259 |
| SCT-Fa060 | EVQLQQSGPVLVKPGTSVKMSCKASGYTFTDYY LNWVKQSHGKSLEWIGVINPYNGGTTYNPKFKG KATLTVDKSSSTAYMELNSLTSEDSAVYYCARD VDYFDNKYVGYAMDYWGQGTSVTVSS | 60 | DWLTQSPATLSVTPGDSVSLSCRASQSISNNL HWYQQKSHESPRLLIKYTSQSISGIPSRFSGSG SGTDFTLSINSVETEDFGMYFCQQSNSWPQYTF GGGTKLEIK | 287 |
| SCT-Fa061 | EVQLQQSGPVLVRPGASVKMSCKASGYSFTGYH MNWVKQSHVKSLEWIGIINPYNSATTYNQNFKG KATLTVDKSSSTAYMELNSLTSEDSWYYCVRG AYGNPLDYWGQGTTLTVSS | 61 | DIKMTQSPSSMYASLGERVTITCKASQDINSYL SWFQLKPGKSPKTLIYRANRLVDGVPSGFSGSG SGQDYSLTISSLEYEDMGIYYCLQYDEYPYTFG GGTKLEIK | 203 |
| SCT-Fa062 | EVQLQQSGTVLARPGASVKMSCKTPGYTFTRYW MHWVKQRPGQGLEWIGAIYPGNSDTNYNQKIKG KAKLTAVTTASTAYMELSSLTNEDSAVYYCTFY YGNYVNYWGQGTTLTVSS | 62 | DVVMTQTPLTLSVTIGQPASISCKSSQSLLDSD GKTYLNWLLQRPGQSPKRLIFLVSKLDSGVPDR FTGSGSGTDFTLKISRVEAEDLGVFYCWQGTHF PTWTFGGGTKLEIK | 310 |
| SCT-Fa063 | EVQLQQSGTVLARPGASVKMSCKTSGYTFIRYW MHWVKQRPGQGLEWIGAIYPGNSDTTYNQKFKG RAKLTAVTSASTAYMELSSLTNEDSAVYYCTFY YGNYVNFWGQGTTLTVSS | 63 | DVVMTQTPLTLSVTIGQPASISCKSSQSLLDSD GKTYLNWLLQRPGQSPKRLIYLVSKLDSGVPDR FTGSGSGTDFTLKISRVEAEDLGVFYCWQGTHF PTWTFGGGTKLEIK | 311 |
| SCT-Fa064 | EVQLQQSGTVLARPGASVKMSCKTSGYTFTRYW MHWVKQRPGQGLEWIGAIYPGNSDTNYNQKIKD KAKLTAVTTASTAYMELSSLTNEDSAVYYCTFF YGNYVNYWGQGTTLTVSS | 64 | DIVMSQSPSSLAVSVGEKVTMSCKSSQSLLYSS NQKNYLAWYQQKPGQSPKLLIYWASTRESGVPD RFTGSGSGTDFTLTISSVKAEDLAVYYCQQYYS YPRTFGGGTKLEIK | 265 |
| SCT-Fa065 | EVQLQQSGTVLARPGASVKMSCKTSGYTFTRYW MHWVKQRPGQGLEWIGAIYPGNSDTNYNQKIKG KAKLTAVTTASTAYMELISLTNEDSAVYYCTFY YGNYVNYWGQGTTLTVSS | 65 | DVVMTQTPLTLSVTIGQPASISCKSSQSLLDSD GKTYLNWLLQRPGQSPKRLIFLVSKLDSGVPDR FTGSGSGTDFTLKISRVEAEDLGVFYCWQGTHF PTWTFGGGTKLEIK | 310 |
| SCT-Fa066 | EVQLQQSGTVLARPGASVKMSCKTSGYTFTRYW MHWVKQRPGQGLEWIGAIYPGNSDTNYNQKIKG KAKLTAVTTASTAYMELSSLTNEDSAAYYCTFY YGNYVNYWGQGTTLTVSS | 66 | DVVMTQTPLTLSVTIGQPASISCKSSQSLLASD GKTYLNWLLQRPGQSPKRLIFLVSKLDSGVPDR FTGSGSGTDFTLKISRVEAEDLGIFYCWQGTHF PTWTFGGGTKLEIK | 308 |
| SCT-Fa067 | EVQLQQSGTVLARPGASVKMSCKTSGYTFTRYW MHWVKQRPGQGLEWIGAIYPGNSDTNYNQKIKG KAKLTAVTTASTAYMELSSLTNEDSAVYYCTFY YGNYVNYWGQGTTLTVSS | 67 | DVVMTQIPLTLSVTIGQPASISCKSSQSLLDSD GKTYLNWLLQRPGQSPKRLIFLVSKLDSGVPDR FTGSGSGTDFTLKISRVEAEDLGVFYCWQGTHF PTWTFGGGTKLEIK | 290 |
| SCT-Fa068 | EVQLQQSGTVLARPGASVKMSCKTSGYTFTRYW MHWVKQRPGQGLEWIGAIYPGNSDTNYNQKIKG KAKLTAVTTASTAYMELSSLTNEDSAVYYCTFY YGNYVNYWGQGTTLTVSS | 67 | DVVMTQTPLTLSVTIGQPASISCKSSQSLLDSD GKTYLNWLLQRPGQSPKRLIFLVSKLDSGVPDR FTGSGSGTDFTLKISRVEAEDLGVFYCWQGTHF PTWTFGGGTKLEIK | 310 |
| SCT-Fa069 | EVQLQQSGTVLARPGASVKMSCKTSGYTFTRYW MHWVKQRPGQGLEWIGAIYPGNSDTNYNQKIQD KAKLTAVTTASTAYMELSSLTNEDSAVYYCTFF YGNYVNYWGQGTTLTVSS | 68 | DVVMTQTPLTLSVTIGQPASISCKSSQSLLDSD GKTYLNWLLQRPGQSPKRLIFLVSKLDSGVPDR FTGSGSGTDFTLKISRVEAEDLGFFYCWQGTHF PTWTFGGGTKLEIK | 309 |
| SCT-Fa070 | EVQLQQSGTVLARPGASVKMSFKTSGYTFTRYW MHWVKQRPGQGLEWIGAIYPGNSDTNYNQKIKG KAKLTAVTTASTAYMELSSLTNDDSAVYYCTFY YGNYVNYWGQGTTLTVSS | 69 | DVVMTQTPLTLSVTIGQPASISCKSSQSLLDSD GKTYLNWLLQRPGQSPKRLIFLVSKLDSGVPDR FTGSGSGTDFTLKISRVEAEDLGVFYCWQGTHF PTWTFGGGTKLEIK | 310 |
| SCT-Fa071 | EVQLQQSVAELVRPGASVKLSCTASGFNIKDFY MHWVHQRTEKGLEWIGRIDPEDGKTKYAPKFQG KATITADTSSNTAYLQLSSLTSEDTAVYYCARS NPYFFDYWGQGTTLTVSS | 70 | DIVLTQSQKFMSASVGDRVSVTCKASQNVGTNV AWYQQRPRQSPKALIYSASYRYSGVPDRFTGSG SGTDFTLAISNVQSEDLAEYFCQQYNSYPYTFG GGTKLEII | 264 |
| SCT-Fa072 | EVQLVESGGDLVKPGGSLKLSCAASGFTFSDYG MSWVRQTPDKRLEWVATISSGGSYTYYADSVKG RFTISRDNAKNTLYLQMSSLKSEDTAMYFCTRH EITTVLSTDYAMDYWGQGTSVTVSS | 71 | DVVMTQTPLSLPVSLGDQASISCRSSQSLVHSI GNTYLHWYLQKPGQSPKLLIYKVSNRFSGVPDR FSGSGSGTDFTLKIIRVEAEDLGVYFCSQSTHI PWTFGGGTKLELR | 300 |
| SCT-Fa073 | EVQLVESGGGLVRPGGSLKLSCAASGFTFSDYG MHWVRQAPEKGLDWIAYISSGSISSGSSTIYYA DTVKGRFTISRDNAKNTLFLQMTSLRSEDTAMY YCARHDWRGLYAMDYWGQGTSVTVSS | 72 | QIVLTQSPAIMSASPGKKVTLTCSASSSVSSSY LYWYQQKPGSSPKLWIYSTSNLASGVPARFSGS GSGTSYSLTISSMEAEDAASYFCHQSSSYPPTF GGGTKLEIK | 324 |
| SCT-Fa074 | EVQLVESGGGLVRPGGSLKLSCVVSGFTFSDYG MHWVPQAPEKGLDWIAYISGGSISSGSSTIYYA DTVKGRFTISRDNAKNTLFLQMTSLRSEDTAMY YCARHDWRGLYAMDYWGQGTSVTVSS | 73 | QIVLTQSPAIMSASPGKKVTLTCSASSSVSSSY LYWYQQKPGSSPKLWIYSTSNLASGVPARFSGS GSGTSYSLTISSMEAEDAASYFCLQWSSYPPTF GGGTKLEIK | 326 |
| SCT-Fa075 | EVQLVESGGGLVRPGGSLKLSCVVSGFTFSDYG MHWVRQAPEKGLDWIAYISGGSISSGSSTIYYA DTVKGRFTISRDNAKNTLFLQMTSLRSEDTAMY YCARHDWRGLYAMDYWGQGTSVTVSS | 74 | QIVLTQSPAIMSASPGKKVTLTCSASSSVSSSY LYWYQQKPGSSPKLWIYSTSNLASGVPARFSGS GSGTSYSLTISSMEAEDAASYFCLQWSSYPPTF GGGTKLEIK | 326 |
| SCT-Fa076 | EVQVLETGGGLVQPGGSRGLSCEGSGFTFSGFW MSWVRQTPGKTLEWIGDINSDGSTIHYGPSMKD RFTIFRDNDKNTLYLQMNNVRSEDTAKYFCMSL LLNVWGTGTTVTVSS | 75 | DIQMTQSPSSLSASLGERVSLTCRASQEISGYL SWLQQKPDGTIKRLISRASSLDFGVPKRFRGSR SGSDYSLTISSLESEDFADYYCLQYANYPLTFG AGTKLELK | 243 |
| SCT-Fa077 | EVRLVESEGGLVQPGSSMKLSCTASGFTFSDYY MAWVRQVPEKGLEWVANINYDGTNTYYLDSLKS RFIISRDNAKNILYLQMSGLKSEDTATYYCGRG IYYYGRSHAMDYWGQGTSVTVSS | 76 | DILMTQSPSSMSVSLGDTVSITCHASQGINNNV GWLQQKPGKSFKGLIYHGTNLEDGVPSRFSGSG SGADYSLTIYSLESEDFADYYCVQYDQFPPTFG GGTKLEIK | 217 |
| SCT-Fa078 | QGQLQQPGAEWKPGAAVKLSCKASGYTFTSYW MQWIKQRPGQGLDWIGEIDPSDNYTDYNQNFKG KATLTVDTSSNTAYLQLSSLTSEDSAVYYCARE GAFSSPFAYWGPGTLVTVSS | 77 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa079 | QGQLQQPGAEVVKPGAAVKLSCKASGYTFTSYW MQWIKQRPGQGLEWIGEIDPSDNYTDYHQNFKG KATLTVDTSSSTAYLHLSSLTSEDSAVYYCARE GAFSSPFAYWGPGTLVTVSS | 78 | DILMTQSPSSMSVSLGDRVSIICHASQDIRSNI GWLQQKPGKSLKGLIYHGTNLEDGVPSRFSGSG SGADYSLTISSLESEDFADYYCVQYVQFPRTFG GGTKLEIK | 211 |
| SCT-Fa080 | QGQLQQPGAEWKPGAAVKLSCKASGYTFTSYW MQWIKQRPGQGLEWIGEIDPSDNYTDYNQHFKG KATLTVDTSSSTAYLQLSSLTSEDSAVYYCARE GAFSSPFAYWGPGTLVTVSS | 79 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa081 | QGQLQQPGAEWKPGAAVKLSCKASGYTFTSYW MQWIKQRPGQGLEWIGEIDPSDNYTDYNQNFKG KATLTVDTSSSTAYLQLSSLTSEDSAVYYCAKE GAFSSPFAYWGPGTLVTVSS | 80 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa082 | QGQLQQPGAEWKPGAAVKLSCKASGYTFTSYW MQWIKQRPGQGLEWIGEIDPSDNYTDYNQNFKG KATLTVDTSSSTAYLQLSSLTSEDSAVYYCARE GAFSSPFAYWGPGTLVTVSS | 81 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa083 | QGQLQQPGAEWKPGAAVKLSCKASGYTFTSYW MQWIKQRPGQGLEWIGEIDPSDNYTDYNQNFKG KATLTVDTSSSTAYLQLSSLTSKDSAVYYCARE GAFSSPFAYWGPGTLVTVSS | 82 | DILMTQSPSSMSVSLGDTVSIICHASQDIRSNI GWLQQKPGKSFKGLIYHGTNLEDGVPSRFSGSG SGADYSLTISSLESEDFADYYCVQYIQFPRTFG GGTKLEIK | 213 |
| SCT-Fa084 | QGQLQQPGAEVVKPGAAVNLSCKASGYTFTSYW MQWIKQRPGQGLEWIGEIDPSDNYTDYNQNFKG KATLTVDTSSSTAYLQLSSLTSEDSAVYYCARE GAFSSPFAYWGPGTLVTVSS | 83 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIN | 215 |
| SCT-Fa085 | QIQLQQSGPELVKPGASVKISCKASGYTFTDYY INWVKQRPGQGLEWIGWIYPGSDNTKYNEKFKG KSTLTVDTSSSTAYMQLSSLTSEDSAVYFCARS GNYYAMDYWGQGTSVTVSS | 84 | SIVMTQTPKFLLVSPGDRVTITCRASQSVSNNV AWYQQRPGQSPKLLIYYASNRYTGVPDRFTGSR YGTDFSFTISTVQAEDLAVYFCQQDYSSPYTFG GGTKLEIK | 332 |
| SCT-Fa086 | QIQLQQSGPELVKPGASVKISCKASGYTFTDYY INWVKQRPGQGLEWIGWIYPGSGNTKYNEKFKG KATLTVDTSSNTAYMQLSSLTSEDSAVYFCARA YDYGFPYWGQGTLVTVSA | 85 | SIVMTQTPKFLLVSAGDRVTITCKASQSMNNGV TWYQQKPGQSPKLLISYASNRYTGVPDRFTGSG YGTDFTFTISAVQAEDLAVYFCQQDYSSPYTFG GGTKLEIK | 328 |
| SCT-Fa087 | QIQLQQSGPELVKPGASVKISCKASGYTFTDYY INWVKQRPGQGLEWIGWIYPGSGNTKYNEKFKG KATLTVDTSSSTAYMHLSSLTSEDSAVYFCARA YDYGFAYWGQGTLVTVSA | 86 | SIVMTQTPKFLLVSAGDRVTITCKASQSVSYGV TWYQQKPGQSPKLLISYSSNRYTGVPDRFTGSG SGTDFTFTISTVQAEDLAVYFCQQDYSSPYTFG GGTKLEIK | 331 |
| SCT-Fa088 | QIQLQQSGPELVKPGASVKISCKASGYTFTDYY INWVKQRPGQGLEWIGWIYPGSGNTKYNEKFKG KATLTVDTSSSTAYMQLSSLTSEDSAVYFCARA YDYGFAYWGQGTLVTVSA | 87 | SIVMTQTPKFLLVSAGDRVTITCKASQSVSHGV TWYQQKPGQSPKLLISYASNRYTGVPDRFTGSG YGTDFTFTISTVQAEDLAVYFCQQDYSSPYTFG GGTKLEIK | 329 |
| SCT-Fa089 | QIQLQQSGPELVKPGASVKISCKASGYTFTDYY INWVKQRPGQGLEWIGWIYPGSGNTKYNEKFKG KSTLTVDTSSSTAYMQLSSLTSEDSAVYFCARS GNYYAMDYWGQGTSVTVSS | 88 | SIVMTQTPKFLLVSPGDRVTITCRASQSVSNNV AWYQQRPGQSPKLLIYYASNRYTGVPDRFTGSR YGTDFSFTISTVQAEDLAVYFCQQDYSSPYTFG GGTKLEIK | 332 |
| SCT-Fa090 | QIQLVQSGPEMKKPGETVKISCKASGYIFRTYG MSWVKQAPGKGLEWMGWINTYSGEPTYADDFKG RFAFSLETSANTAYLQINNLKNEDTATFFCARA QHYFDWWGQGTTLTVSS | 89 | DIKMTQSPSSMYASLGERVTITCKASQDIDSYL NWFQQKPGKSPKTLIYRATRLVDGVPSRFSGSG SGQDYSLTISSLEYEDMGIYYCLQYDEFPYTFG GGTKLEIK | 198 |
| SCT-Fa091 | QIQLVQSGPEMKKPGETVKISCKASGYTFRTYG MSWVKQAPGKGLEWMGWINTYSGEPTYADDFKG RFAFSLETSANTAYLQINNLKNEDTATFFCARA QHYFDWWGQGTTLTVSS | 90 | DIKMTQSPSSMYASLGERVTITCKASQDIDRYL NWFQQKPGKSPKTLIYRATRLVDGVPSRFSGSG SGQDYSLTISSLEYEDMGIYYCLQYDEFPYTFG GGTKLEIK | 197 |
| SCT-Fa092 | QIQLVQSGPEMKKPGETVKISCKASGYTFRTYG MSWVKQAPGKGLEWMGWINTYSGEPTYADDFKG RFAFSLETSANTAYLQINNLKNEDTATFFCARA QHYFDWWGQGTTLTVSS | 90 | DIKMTQSPSSMYASLGERVTITCKASQDIDSYL NWFQQKPGKSPKTLIYRATRLVDGVPSRFSGSG SGQDYSLTISSLEYEDMGIYYCLQYDEFPYTFG GGTKLEIK | 198 |
| SCT-Fa093 | QVHLKQSGAELVRPGTSVKLSCKASGYTLTDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 91 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa094 | QVLLQQPGAELVRPGTSVKLSCKASGYTFTNFW MHWVKQRPGQGLEWIGVIDPSDISTHYNQNFKG KAILTVDTSSSSAYMQLSSLTSEDSAVYYCARN DYDGGYFDYWGQGTTLTVSS | 92 | DVVMTQTPLSLPVSLGDQASISCRSSQSLVHGN GNTYLHWYLQKPGQSPKLLIYTVSNRFSGVPDR FSGSGSGTDFTLKISRVEAEDLGVYFCSQTIHA PWTFGGGTKLEIK | 299 |
| SCT-Fa095 | QVQLKESGPGLVAPSQSLSITCTVSGFSLTSSV ISWVRQPPGKGLEWLGIIWAGGDTNYNSALKSR LSISKDNSKSQVFLKMNSLQTDDTARYYCARNL NAMDYWGQGTSVTVSS | 93 | DVLMTQTPLSLTVSLGDQASISCRSSQSIVHSN GNTYLEWFLQKPGQSPKLLIYKVSNRFSGVPDR FSGSGSGTDFTLKISRVEAEDLGVYYCFQGSHV PWTFGGGTKLEIK | 285 |
| SCT-Fa096 | QVQLKQSGAELVRPGTSIKLSCKASGYTFTDFY INWVKQRPGQGLEWIARIYPGGANTYYNDNFKG KATLTAEKSSITAYMQLSSLTSEDSAVYFCARG ANWAFASWGQGTLVTVSA | 94 | DIVMTQSHKFMSTSVGDRVSITCKASQGVSTAV AWYQQEPGQSPKLLIYSASYRYTGVPDRFTGSG SGTDFTFTISSVQAEDLAVYYCQQYYNTPYTFG GGTKLEIK | 267 |
| SCT-Fa097 | QVQLKQSGAELVRPGTSVKLSCKASGYILTDYY INWVKQRPGQGLEWIARIYPGNGNTYYNEKFKG KATLTAEKSSSTVNMQVSSLTSEDSAVYFCARG GSSDRIYYYAMDHWGQGTSVTVSS | 95 | DIQMNQSPSSLSASLGDTINITCHASQNINVWL SWYQQKPGNIPKLLIYKASNLHTGVPLRFSGSG SGTGFTLTISSLQPEDIATYYCQQGQSYPLTFG SGTKLEIK | 222 |
| SCT-Fa098 | QVQLKQSGAELVRPGTSVKLSCKASGYILTDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 96 | DIQMNQSPSSLSASLGDTITITCHASQNINVWL SWYQQKPGNIPKLLIYKASNLHTGVPSRFSGSG SGTGFTLAISSLQPEDIATYYCQQGQSYPLTFG SGTKLALK | 227 |
| SCT-Fa099 | QVQLKQSGAELVRPGTSVKLSCKASGYILTDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 96 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa100 | QVQLKQSGAELVRPGTSVKLSCKASGYTLIDYY INWVKQRPGQGLEWIARIYPGNGNTYYNEKFKG KATLTAEKSSSTVNMQVSSLTSEDSAVYFCARG GSSDRIYYFAMDYWGQGTSVTVSS | 97 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa101 | QVQLKQSGAELVRPGTSVKLSCKASGYTLIDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 98 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa102 | QVQLKQSGAELVRPGTSVKLSCKASGYTLNDYY INWVKQRPGQGLEWIARIYPGNNNTYYNEKFKG KATLTAEKSSNTAYMQVSSLTSEDSAVYFCARG GSSVRVYYYAMDYWGQGTSVTVSS | 99 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa103 | QVQLKQSGAELVRPGTSVKLSCKASGYTLTDYY INWVKQRPGQGLEWIARIYPGNGNTLYNAKFEG KATLTAERSSSTAYMQVSSLTSEDSAVYFCARG GSSVRVYYYAMDFWGQGTSVTVSS | 100 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa104 | QVQLKQSGAELVRPGTSVKLSCKASGYTLTDYY INWVKQRPGQGLEWIARIYPGNGNTYYNEKFKC KATLTAEKSSSTVNMQVTSLTSEDSAIYFCARG GSSDRIYYYAMDYWGQGTSVTVSS | 101 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEVK | 230 |
| SCT-Fa105 | QVQLKQSGAELVRPGTSVKLSCKASGYTLTDYY INWVKQRPGQGLEWIARIYPGNGNTYYNEKFKG KATLTAEKSSNTVNMQVSSLTSEDSAVYFCARG GSSDRIYYYAMDYWGQGTSVTVSS | 102 | DIQMNQSPSSLSASLGDTITITCHASQNINVWL SWYQQKPGDIPKLLIYKASNLHTGVPSRFSGSG SGTGFTLTISSLQPEDIATYYCQQGQSYPLTFG SGTKLEIK | 225 |
| SCT-Fa106 | QVQLKQSGAELVRPGTSVKLSCKASGYTLTDYY INWVKQRPGQGLEWIARIYPGNGNTYYNEKFKG KATLTAEKSSSTAYMQVSSLTSEDSAVYFCARG GSSVRVYYYAMDYWGQGTSVTVSS | 103 | DIQMNQSPSSLSASLGDTITITCHASQNINVWL SWYQQKPGNIPKLLIYKASNLHTGVTSRFSGSG SGTGFTLTISSLQPEDIATYYCQQGQSYPLTFG SGTKLEIK | 234 |
| SCT-Fa107 | QVQLKQSGAELVRPGTSVKLSCKASGYTLTDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 104 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa108 | QVQLKQSGAELVRPGTSVKLSCKASGYTLTDYY INWVKQRPGQGLEWIARIYPGNGNTYYNEKFKG KATLTAEKSSSTVNMQVTSLTSEDSAIYFCARG GSSDRIYYYAMDYWGQGTSVTVSS | 105 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEVK | 230 |
| SCT-Fa109 | QVQLKQSGAELVRPGTSVKLSCKASGYTLTDYY INWVKQRPGQGLEWIARIYPGNGNTYYNEKFKG KATLTAEKSSSTVNMQVTSLTSEDSAIYFCARG GSSDRIYYYAMDYWGQVTSVTVSS | 106 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEVK | 230 |
| SCT-Fa110 | QVQLKQSGAELVRPGTSVRLSCKASGYTLTDYY INWVKQRPGQGLEWIARIYPGNGNTYYNEKFKG KATLTAEKSSSTVNMQVTSLTSEDSAVYFCARG GSSDRIYYYAMDYWGQGTSVTVSS | 107 | DIQMNQSPSSLSASLGDTITITCHASQNINVWL SWYQQKPGNIPKLLIYKASNLHTGVPSRFSGSG SGTGFTLTISSLQPEDIATYYCQQGQSYPLTFG SGTKLEIK | 229 |
| SCT-Fa111 | QVQLKQSGPGLVQPSQSLSITCTVSGFSLISYG VHWVRQSPGKGLEWLGIIWSGGNTDYNAAFISR LSISKDNSKSQVFFKLNSLQADDTAIYYCARTA NALDYWGQGTSVTVSS | 108 | DVLMTQTPLSLPVSLGDQASISCRSSQTIVHSN GNTYLEWYLQKPGQSPKLLIYKVSNRFSGVPDR FSGSGSGTDFTLKISRVEAEDLGVYYCFQGSHV PWTFGGGTKLEIK | 282 |
| SCT-Fa112 | QVQLQQPGADWKPGASVKLSCKASGYTFTNYW IQWIKQRPGQGLEWIGEIDPSDNYTDYNQNFKG KATMTVDTSSSTAYLQLSSLTSEDSAVYFCAGE GAFSSPFAYWGPGTLVTVSS | 109 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa113 | QVQLQQPGAEFVKPGASVKLSCEASGNTFTSYW IQWVKQRPGQGLEWIGEIDPSDSYIDYNQKFKD KATLTVDTSSSTAYMHLSSLTSEDSAVYYCARE GAYSSPFAYWGQGTLVTVSA | 110 | DILMTQSPSSMSVSLGDTISIICHASQDIRSNI GWLQQKPGKSFKGLIYHGSNLEDGVPSRFSGSG SGADYSLTISSLESEDFADYYCVQYIQFPRTFG GGTKVEIK | 212 |
| SCT-Fa114 | QVQLQQPGAEFVKPGASVKLSCKASGYTFTSYW MHWMKQRPGRGLEWIGRIDPDSGGTKYNEKFKN KATLTVDKPSSTAYMQLSSLTSEESAVYYCARY YWFFDIWGTGTTVTVSS | 111 | DIVMTQSHKFMSTSVGNRVSITCKVSQDVSTAV AWYQQKPGQSPKLLIYSASYRYTGVPDRFAGSG SGTDFTFTISSVQAEDLAVYYCQQHFNTPFTFG SGTNLEIK | 268 |
| SCT-Fa115 | QVQLQQPGAELVKPGASVKLSCKASCYTFISYW MHWMKQRPGRGLEWIGRIDPDSGGTKYNEKFKS KATLTVDKPSSTAYMQLSSLTSEDSAVYYCARY YWFFDIWGTGTTVTVSS | 112 | DIVMTQSHKFMSTSVGDRVSITCKASQDVGAAV AWYQQKPGQSPKLLIYSASYRYTGVPDRFAGSG SGTDFTFTISSVQAEDLAVYYCQQHFNTPFTFG SGTNLEIK | 266 |
| SCT-Fa116 | QVQLQQPGAELVKPGASVKLSCKASGYSFISSW MEWVKQRPGQGLEWIGRIHPSDSDTNYNQKFKA KATLTVDKSSSTAYMQLSSLTSEDSAVYYCAII HRGFYFAMDYWGQGTSVTVSS | 113 | DVLMTQTPLSLPVSLGDQASISCRSSQSIVHSN GNTYLEWYLQKPGQSPKLLIYKVSNRFSGVPDR FSGSGSGTDFTLKISRVEAEDLGVYYCFQGSHV PWTFGGGTKLEIK | 280 |
| SCT-Fa117 | QVQLQQPGAELVKPGASVKLSCKASGYTFISYW MHWMKQRPGRGLEWIGRIDPDSGGTKYNEKFKS KATLTVDKPSSTAYMQLSSLTSEDSAVYYCARY YWFFDIWGTGTTVTVSS | 114 | DIVMTQSHKFMSTSVGDRVSITCKASQDVGAAV AWYQQKPGQSPKLLIYSASYRYTGVPDRFAGSG SGTDFTFTISSVQAEDLAVYYCQQHFNTPFTFG SGTNLEIK | 266 |
| SCT-Fa118 | QVQLQQPGAELVKPGASVKLSCKASGYTFISYW MHWVKQRPGRGLEWIGRIDPNSGDTKYNEKFKS KATLTVDKPSSTAYMQLSSLISEDSAVYYCARW GWDAFAYWGQGTLVTVSA | 115 | DAVMTQTPLSLPVSLGDQASISCRSSQSLENSD GNTYLNWYFQKTGQSPQLLIYRVSNRFSGVLDR FSGSGSGTDFTLKISRVEAEDLGVYFCLQVTHV PPTFGAGTKLELK | 187 |
| SCT-Fa119 | QVQLQQPGAELVKPGASVKVSCKASGYSFISSW MEWVKQRPGQGLEWIGRIHPSDSDTNYNQKFKG KATLTVDKSSSTAYMQLSSLTSEDSAVYYCAII HRGFYFAMDYWGQGTSVTVSS | 116 | DVLMTQTPLSLPVSLGDQASISCRSSQSIVHSN GNTYLEWYLQKPGQSPKLLIYKVSNRFSGVPDR FSGSGSGTDFTLKISRVEAEDLGVYYCFQGSHV PWTFGGGTKLEIK | 280 |
| SCT-Fa120 | QVQLQQPGAELVKPGASVKVSCKASGYSFISYW MYWVKQRPGQGLEWIGRIHPSDSDTNYNQKYKG KATLTVDRSSSTAYMQLSSLTSEDSAVYYCTIS HYSGSSPETYWGQGTTLTVSS | 117 | DVLMTQTPLSLPVSLGDQASISCRSSQSIVHSN GNTYLEWYLQKPGQSPKLLIYKVSNRFSGVPDR FSGSGSGTDFTLKISRVEAEDLGVYYCFQGSHV PYTFGGGTKLEIK | 281 |
| SCT-Fa121 | QVQLQQPGAELVKPGASVKVSCKASGYTFISHW MYWVRQRPGQGLEWIGRIHPSDSDTDYSQEFKG KATLTVDKSSSTAHMQLSSLTSEDSAVYYCAIW SYYYGSRLDYWGQGTTLTVSS | 118 | DVLMTQAPPSLPVSLGDQASISCRSSQNIVHSN GNTYLEWYLQRPGQSPKLLIYKVSNRFSGVPDR FSGSGSGTDFTLKISRVEAEDLGVYYCCQGSHV PYTFGGGTKLEIK | 278 |
| SCT-Fa122 | QVQLQQPGAELVMPGASVKLSCEASGYTFTTFW IHWVKQRPGQGLEWIGEIDPSDSYTNYNQNFKG KATLTVDKSSSTAYLQLSSLTSEDSAVYYCARN YGSNYVGYFDYWGQGTTLTVSS | 119 | DIQMTQTTSSLSASLGDRVTISCRASQDISNYL NWYQQKPDGTVKLLIYYTSRLHSGVPSSFSGSG SGTDYSLTISNLEQEDIAIYFCQQANALPWTFG GGTKLEIK | 250 |
| SCT-Fa123 | QVQLQQPGAELVMPGASVKLSCKASGYTFNTYW MHWVKQRPGQGLEWIGEIDPSDSYTNYNQNFNG KATLTVDKSSSTAYLQLSSLTSEDSAVYYCARN YGSNYVGYFDYWGQGTTLTVSS | 120 | DIQMTQTTSSLSASLGDRVTISCRASQDISNYL NWYQQKPDGTVKLLIYYTSRLPSGVPSSFSGSG SGTDYSLTISNLEQEDIATYFCQQAKALPWTFG GGTKLEIK | 253 |
| SCT-Fa124 | QVQLQQPGAELVMPGASVKLSCKASGYTFSTYW MHWVKQRPGQGLEWIGEIDPSDSYTNYNQNFKG KATLTVDKSSSTAYMQLSSLTSEDSAVYYCARN YGSNYVGYFDHWGRGTTLTVSS | 121 | DIQMTQTTSSLSASLGDRVTISCRASQDISNYL NWYQQKPDGTVKLLIYYTSRLHSGVPSSFSGSG SGTDYSLTISNLAQEDIATYFCQQGNALPWTFG GGTKLEIK | 249 |
| SCT-Fa125 | QVQLQQPGAELVMPGASVKLSCKASGYTFTSYW MHWVKQRPGQGLEWIGEIDPSDNYTNYNQKFKG KATLTVDGSSSTAYMQLSSLTSEDSAVYYCARN YGSNYVGYFDYWGQGTTLTVSS | 122 | DIQMTQTTSSLSASLGDRVTISCRASQGISNYL NWYQQKPDGTVKLLIYYTSRFHSGVPSRFSGSG SGTDYSLTISNLEQEDIATYFCQQGNSLPWTFG GGTKLEIK | 256 |
| SCT-Fa126 | QVQLQQPGAELVMPGASVKLSCKASGYTFTSYW MHWVKQRPGQGLEWIGEIDPSDSYSNYNQKFKG KATLTVDKSSSTAYMQLISLTSEDSAVYYCARN YGSNYVGYFDYWGQGTTLTVSS | 123 | DIQMTQTTSSLSASLGDRVTISCRASQDISNYL NWYQQKPDGTVKLLIYYTSRLLSGVPSRFSGSG SGTDYSLTISNLEQEDIATYFCQQGNTLPWTFG GGTNLEIK | 252 |
| SCT-Fa127 | QVQLQQPGAELVMPGASVKLSCKASGYTFTTYW MHWVKQRPGQGLEWIGEIDPSDGYSNYNQNFKG KATLTVDKSSSTAYLQLSSLTSEDSAVYYCARN YGSNYVGYFDYWGQGTTLTVSS | 124 | DIQMTQTTSSLSASLGDRVTISCRSSQDISNYL NWYQQKPDGTVKLLIYYTSRLHSGVPSSFSGSG SGTDYSLTISNLEQEDIATYFCQQANALPWTFG GGTKLEIK | 257 |
| SCT-Fa128 | QVQLQQPGAELVMPGASVKLSCKASGYTFTTYW MHWVKQRPGQGLEWIGEIDPSDSYSNYNQKFKG KATLTVDKSSSTAYMQLSSLTSEDSAVYYCARN YGSNYVGYFDYWGQGTTLTVSS | 125 | DIQLTQTTSSLSASLGDRVTINCRASQDISNYL NWYQQKPDGTVKLLIYYTSRLLSGVPSSFSGSG SGTDYSLTISSLEQEDIATYFCQQGKALPWTFG GGTKLEIK | 219 |
| SCT-Fa129 | QVQLQQPGAELVMPGASVKLSCKASGYTFTTYW MHWVKQRPGQGLEWIGEIDPSDSYTNYNQKFKG KATLTVDKSSSTAYMQLSSLTSEDSAVYYCARN YGSNYVGYFDYWGQGTTLTVSS | 126 | DIQLTQTTSSLSASLGDRVTINCRASQDISNYL NWYQQKPDGTVKLLIYYTSRLLSGVPSSFSGSG SGTDYSLTISSLEQEDIATYFCQQGKALPWTFG GGTKLEIK | 219 |
| SCT-Fa130 | QVQLQQPGAELVMPGASVRLSCKASGYTFTTYW MHWVRQRPGQGLEWIGEIDPSDSYTNYNQNFKG KATLTVDKSSSTAYLQLSSLTSEDSAVYYCARN YGSNYVGYFDHWGQGTTLTVSS | 127 | DIQMTQTTSSLSASLGDRVTISCRASQDISNYL NWYQQKPDGTVKLLIYYTSRLHSGVPSSFSGSG SGTDYSLTISNLEQEDIATYFCQQANALPWTFG GGTKLEIK | 251 |
| SCT-Fa131 | QVQLQQPGAELVMPGASVRLSCKTSGYTFSTYW MHWVKQRPGQGLEWIGEIDPSDSYTNYNQNFKG KATLTVDKSSSTAYMKLSSLTSEDSAVYYCARN YGSNYVGYFDYWGQGTTLTVSS | 128 | DIQMTQTASSLSASLGDRVTISCRASQDINNYL NWYQQKPDGTVKLLIYYTSRLHSGVPSSFSGSG SGTDYSLTISNLEQEDIATYFCQQGNALPWTFS GGTKLEIK | 245 |
| SCT-Fa132 | QVQLQQPGAELVMPRASVKLSCKASGYTFTSYW MHWVKQRPGQGLEWIGEIDPSDGYTNYNHKFKG KATLTVDKSSSTAYMQLSSLTSEDSAVYYCARN YGSNYVGYFDYWGQGTTLTVSS | 129 | DIQMTQTTSSLSASLGDRVTISCRASQDISNYL NWYQQKRDGTVKLLIYYTSRFHSGVPSRFSGSG SGTDYSLTISNLEQEDIATYFCQQGNTLPWTFG GGTKLEIK | 254 |
| SCT-Fa133 | QVQLQQPGAELVMSGASVKLSCKASGYTFTTYW MHWVRQRPGQGLEWIGEIDPSDSYSNNNQNFKA KATLTVDKSSSTAYMQLGSLTSEDSAVYYCARN YGSNYVGYFDYWGQGTTLTVSS | 130 | DIQMTQSTSSLSASLGDRVTISCRASQDITNYL NWYQQKPDGTVKLLIYHTSRLHAGVPSRFSGSG SGTDYSLTISNLDQEDIATYFCQQGNALPWTFG GGTKLEIK | 244 |
| SCT-Fa134 | QVQLQQPGAELVRPGSSVKLSCKASGYNFTNYW IDWVKQRPGQGLAWIGDIYPSDNKTHYNQNFKG KATLTVDKSSNTAYMQLSSLTSEDSAVYYCAAS MDYWGQGTSVTVSS | 131 | DIVMTQSPSSLTVTAGEKVTMSCKSSQSLLNSK NQKNFLTWYQQKPGQPPKLLIYWASTRESGVPD RFTGSASGTDFTLTISSVQAEDLAVYYCQNDYS YPYTFGGGTKLEIK | 272 |
| SCT-Fa135 | QVQLQQPGAELVRPGSSVKLSCKASGYNFTSYW IDWVKQRPGQGLEWIGDIYPSDSKTHYNQNFKG KATLTVDKSSNTAYMQLSNLTSEDSAVYYCATS MDYWGQGTSVTVSS | 132 | DIVMTQSPSSLTVTAGEKVTMSCKSSQSLLNSK NQKNFLTWYQQKPGQPPKLLIYWASTRESGVPD RFTGSASGTDFTLTISSVQAEDLAVYYCQNDYD YPYTFGGGTKLEIK | 271 |
| SCT-Fa136 | QVQLQQPGAELVRPGTSVKLSCKASGYTFINYW MHWVKQRPGQGLEWIGVIDPSHTSTSYNQKFKG KATLTVDTSSTTTSMQLTSLTSEDSAVYYCARN DYDGGYFDYWGQGTTLTVSS | 133 | DVVMTQIPLSLPVSLGDQASISCRSSQSLVHSN GNTYLHWYLQKPGQSPKLLIYTVSNRFSGVPDR FSGSGSGTDFTLKISRLEAEDLGVYFCSQTTHV PWTFGGGTKLEIK | 289 |
| SCT-Fa137 | QVQLQQPGAELVRPGTSVKLSCKASGYTFSRYW IHWVKQRPGQGLEWIGVIDPSDNNTHYNQKFKG KATLTVDASSSTAYMQLSSLTSEDSAVFFCARN DYDGGYFDYWGQGTTLTVSS | 134 | DVVMTQTPLSLPVSLGDQASISCRSSQSLVHSN GNTYLHWYLQKPGQSPKLLIYKVSNRFSGVPDR FSGSGSGTDFTFKISRVEAENLGVYFCSQSTHV PWTFGGGTKLEIK | 301 |
| SCT-Fa138 | QVQLQQPGAELVRPGTSVKLSCKASGYTFTRYW MHWVRQRPGQGLEWIGVIDPSSSSTDYNQKFKG KATLTVDTSSSTAYMQLSSLTSEDSAVYYCARN DYDGGYFDYWGQGTTLTVSS | 135 | DVVMTQSPLSLPVSLGDQASISCRSSQSLVHSN GITYLHWYLQKPGQSPNLLIYKVSNRFSGVPDR FSGSGSGTDFTLKISRVEAEDLGVYFCSQTTHV PWTFGGGTKLEIK | 292 |
| SCT-Fa139 | QVQLQQPGAELVRPGTSVKLSCKASGYTFTSYW MHWVNQRPGQGLEWIGVIDPSDSSTDYNQKFKG KATLTVDTSSSTAYMQLSSLTSEDSAVYYCARN DYDGGYFDYWGQGTTLTVSS | 136 | DFVMTQTPLSLPVSLGDQASISCRSSQSLLHSN GNTYLHWYLQKPGQSPKLLIYKVSNRFSGVPDR FSGGGSGTDFTLKISRVEAEDLGVYFCSQTTHV PWTFGGGTKLEIK | 195 |
| SCT-Fa140 | QVQLQQPGAELVRPGTSVKLSCKASGYTFTSYW MHWVRQRPGQGLEWIGVIDPSSSSTDYNQKFKG KATLTVDTSSSTAYMQLSSLTSEDSAVYYCARN DYDGGYFDYWGQGTTLTVSS | 137 | DVVMTQSPLSLPVSLGDQASISCRSSQSLVHSN GITYLHWYLQKPGQSPKLLIYKVSNRFSGVPDR FSGSGSGTDFTLKISRVEAEDLGVYFCSQSTHV PWTFGGGTKLELK | 291 |
| SCT-Fa141 | QVQLQQPGAELVRPGTSVNLSCKASGYTFTSYW MHWVKQRPGQGLEWLGVIDPSDSNTNYNQKFKG KATLTVDTSSSTAYMQLSSLTSEDSAVYYCSRN DYDGGYFDYWGQGTTLTVSS | 138 | DVVMTQTPLSLPVSLGDQASISCRSSQSLVHSN GNTYLHWYLQKPGQSPKLLIYTVSNRFSGVPDR FSGSGSGTDFTLKISRVEAEDLGIYFCSQSTHV PWTFGGGTKLEIK | 304 |
| SCT-Fa142 | QVQLQQPGAELVRPGTSVRLSCKASGYSFTIYW MHWVKQRPGQGLEWIGVIDPSTSNTDYNQKFKG KATLTVDTSSSTAYMQLRSLSSEDSAVYYCARN DYDGGYFDYWGQGTTLTVSS | 139 | DVVMTQTPLSLPVSLGDQASISCRSSQRLVHSN GNTYLHWYLQKTGQSPKLLIYKVSNRFSGVPDR FSGSGSGTDFTLKISRVEAEDLGVYFCSQSTHV PWTFGGGTKLEIK | 296 |
| SCT-Fa143 | QVQLQQPGAELVRSGTSVKLSCKASGYTFISYW MHWVKQRPGQGLEWIGVIDPSDTNTNYNQKFRG KATLTVDTSSSTAYMQLSSLTSEDSAVYFCARN DYDGGYFDYWGQGTTLTVSS | 140 | DVVMTQTPLSLPGSLGDQASISCRSSQSLVHSN GNTYLHWYLQKSGQSPKLLIYKVSNRFSGVPDR FSGSGSGTDFTLKISRVEAEDLGLYFCSQTTHV PWTFGGGTKLEIK | 294 |
| SCT-Fa144 | QVQLQQPGAELVTPGASVKLSCKASGYTFTTYW MHWVKQRPGQGLEWIGEIDPSDSYRNYNQKFKG KATLTVDKSSSTAYMQLSSLTSEDSAVYYCARN YGSNYVGYFDYWGQGTALTVSS | 141 | DIQMTQTTSSLSASLGDRVTISCRASQDITNYL NWYQQRPDGTVKLLIYYTSRLHSGVPSRFSGSG SGTDYSLTISNLAQGDIATYFCQQASTLPWTFG GGTKLDIK | 255 |
| SCT-Fa145 | QVQLQQPGAEWKPGASVKLSCKASGYTFTNYW MQWVRQRPGQGLEWIGEIDPSDNYTDYNQKFKG KATLTVDASSSTAYMQLSSLASEDSAVYFCARE GAFSSPFAFWGPGTLVTVSA | 142 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa146 | QVQLQQPGAEWKPGASVKLSCKASGYTFTNYW VQWVKQRPGQGLEWIGEIDPSDNYTDYNQNFKG KATLTVDTSSSTAYLHLSSLTSEDSAVYYCARE GAFSSPFAYWGPGTLVTVSS | 143 | DILMTQSPSSMSVSLGDRVSIICHASQDIRSNI GWLQQKPGKSLKGLIYHGTNLEDGVPSRFSGSG SGADYSLTISSLESEDFADYYCVQYVQFPRTFG GGTKLEIK | 211 |
| SCT-Fa147 | QVQLQQPGAEWKPGASVKLSCKASGYTFTSYW MQWIKQRPGQGLEWIGEIDPSDNYTDYNQNFKG KATLTVDTSSSTAYLQLSSLASEDSAVYCCARE GAFSSPFAYWGPGTLVTVSS | 144 | DILMTQSPSSMSVSLGDTVSIICHASQDIRSNI GWLQQKPGKSFKGLIYHGTNLEDGVPSRFSGSG SGADYSLTISSLESEDFADYYCVQYVQFPRTFG GGTKLEIK | 214 |
| SCT-Fa148 | QVQLQQPGAEWKPGASVKLSCKASGYTFTSYW MQWVKQRPGQGLEWIGEIDPSDNYTDYNQNFKG KATLTVDTSSSTAYMQLSSLTSEDSAVYYCAKE GAYSSPFAYWGPGTLVTVSA | 145 | DILMTQSPSSMSVSLGDTVTIICHASQDIRSNI GWLRQKPGTSFKGLIYHGTNLEDGVPSRFSGSG SGADYSLTISSLESEDFADYYCVQYVQFPRTFG GGTKLEIK | 218 |
| SCT-Fa149 | QVQLQQPGAEWKPGASVKLSCKASGYTFTTYW MQWVKQRPGQGLEWIGEIDPSDNYTDYNQNFKG KATLTVDTSSNTAFMHLSSLTSEDSAVYYCVRE GAFSSPFAYWGPGTLVTVSA | 146 | DILMTQSPSSMSVSLGDTVSIICHASQDIRSNI GWLQQKPGKSFKGLIYHGTNLEDGVPSRFSGSG SGTDYSLTISSLESEDFAEYYCVQYVQFPRTFG GGTKLEIK | 216 |
| SCT-Fa150 | QVQLQQPGAEWKPGASVKVSCKASGYSFISHW MYWVRQRPGQGLEWIGRIHPSDSDTDYSQEFKG KATLTVDKSSSTAHMQLSSLTSEDSAVYYCAIW SYYYGTRLDYWGQGTTLTVSS | 147 | DVLMTQTPPSLPVSLGDQASISCRSSQNIVHSN GNTYLEWYLQRPGQSPKLLIYKVSNRFSGVPDR FSGSGSGTDFTLKISRVEAEDLGVYYCCQGSHV PYTFGGGTKLEIK | 286 |
| SCT-Fa151 | QVQLQQPGSELVRPGTSVKLSCKASGYTFTSYW MHWVKQRPGQGLEWIGVIDPSDNNTHYNQKFTG KATLTVDTSSSTAYMHLSSLTSEDSAVFYCARN DYDGGYFDFWGQGTTLTVSS | 148 | DVVMTQTPLSLPVSLGDHASISCRSSQSLVHSN GHTYLHWYLQKPGQSPKLLIDKVSNRFSGVPDR FSGSGSGTDFTLKISRVEAEDLGVYFCSQSTHV PWTFGGGTKLEIK | 295 |
| SCT-Fa152 | QVQLQQPGTELVKPGASVKVSCKASGYTFISYC MHWVKQRPGQGLDWIGNIDPNNGGPNYNEKFKT KATLTVDKSSSTAYMQLSSLTSEDSAVYYCARW GWDGFAYWGQGTLVTVSA | 149 | DAVMTQTPLSLPVSLGDQASISCRSSQSLENSN GNTYLNWYLQKPGQSPQLLIYRVSNRFSGVLDR FSGSGSGTDFTLKISRVEAEDLGVYFCLQVTHV PYTFGGGTKLEIK | 190 |
| SCT-Fa153 | QVQLQQPGTELVKPGASVKVSCKASGYTFISYW MHWVKQRPGQGLDWIGNIDPNNGGTNYNEKFKS KATLTVDKSSSTAYMQLSSLTSEDSAVYYCARW GWDGFAYWGQGTLVTVSA | 150 | DAVMTQTPLSLPVSLGDQASISCRSSQSLENSY GNTYLNWYLQKPGQSPQLLIYRVSTRFSGVLDR FSGSGSGTDFTLKISRVEAEDLGVYFCLQVTHV PYTFGGGTKLEIK | 193 |
| SCT-Fa154 | QVQLQQPGTELVKPGASVKVSCKASGYTFISYW MHWVKQRPGQGLEWIGNIDPNSGGTKYNEKFKS KAILTVDKSSSTAYMQLSSLTSEDSAVYYCTRW GWDGFAYWGQGTLVTVSA | 151 | DAVMTQTPLSLPVSLGDQVSISCRSSQSLENSN GNTYLNWYLQKPGQSPQLLIYRISTRFSGVLDR FSGSGSGTDFTLKISRVEAEDLGVYFCLQVTHV PYTFGGGTKLEIK | 194 |
| SCT-Fa155 | QVQLQQPGTELVKPGASVKVSCKASGYTFISYW MHWVKQRPGQGLEWIGNIDPNSGGTNYNEKFKS KAILTVDKSSSTAYMQLSSLTSEDSAVYFCARW GWDGFAYWGQGTLVTVSA | 152 | DVVMTQTPLSLPVSLGDQASISCRSSQSLENSN GNTYLNWYLQKPGQSPQLLIYRISTRFSGVLDR FSGSGSGTDFTLKISRVEAEDLGVYFCLQVTHV PYTFGGGTKLEIK | 297 |
| SCT-Fa156 | QVQLQQPGTELVKPGASVKVSCKASGYTFISYW MHWVKQRPGQGLEWIGNIDPNSGGTNYNEKFKS KAILTVDKSSSTAYMQLSSLTSEDSAVYYCTRW GWDGFAYWGQGTLVTVSA | 153 | DAVMTQTPLSLPVSLGDQVSISCRSSQSLENSN GNTYLNWYLQKPGQSPQLLIYRISTRFSGVLDR FSGSGSGTDFTLKISRVEAEDLGVYFCLQVTHV PYTFGGGTKLEIK | 194 |
| SCT-Fa157 | QVQLQQPGTELVKPGASVKVSCKASGYTFISYW MHWVKQRPGQGLEWIGNIDPNSGITNYNEKFKS KAILTVDKSSSTAYMQLSSLTSEDSAVYYCARW GWDGFAYWGQGTLVTVSA | 154 | DVVMTQTPLSLPVSLGDQASISCRSSQSLENSN GNTYLNWYLQKPGQSPQLLIYRISTRFSGVLDR FSGSGSGTDFTLKISRVEAEDLGVYFCLQVTHV PYTFGGGTKLEIK | 297 |
| SCT-Fa158 | QVQLQQPGTELVKPGASVKVSCKASGYTFISYW MHWVKQRPGQGLEWIGNIDPNSGTTNYNEKFKT KATLTVDKSSSTAYMQLSSLTSEDSAVYYCARW GWDGFAYWGQGTLVTVSA | 155 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQVTHV PYTFGGGTKLEIK | 192 |
| SCT-Fa159 | QVQLQQPGTELVKPGASVKVSCKASGYTFISYW MHWVKQRPGQGLEWIGNIDPNYGGPNYNEKFKT KATLTVDKSSSTAYMQLSSLTSEDSAVYYCARW GWDGFAYWGQGTLVTVSA | 156 | DAVMTQTPLSLPVSLGDQASISCRSSQSLENSN GNTYLNWYLQKPGQSPQLLIYRVSNRFSGVLDR FSGSGSGTDFTLKISRVEAEDLGVYFCLQITHV PYTFGGGTKLEIK | 188 |
| SCT-Fa160 | QVQLQQPGTELVKPGASVKVSCKASGYTFISYW MHWVKQRPGQGLEWIGNIDPNYGGPNYNEKFKT KATLTVDKSSSTAYMQLSSLTSEDSAVYYCARW GWDGFAYWGQGTLVTVSA | 156 | DAVMTQTPLSLPVSLGDQASISCRSSQSLENSN GNTYLNWYLQKPGQSPQLLIYRVSNRFSGVLDR FSGSGSGTDFTLKISRVEAEDLGVYFCLQLTHV PYTFGGGTKLEIK | 189 |
| SCT-Fa161 | QVQLQQPGTELVKPGASVKVSCKASGYTFISYW MHWVKQRPGQGLEWIGNIDPNYGGPNYNEKFKT KATLTVDKSSSTAYMQLSSLTSEDSAVYYCARW GWDGFAYWGQGTLVTVSA | 156 | QIVLTQSPALMSASPGEKVTMTCSASSSVSYMY WYQQKPRSSPKPWIYLTSNLASGVPARFSGSGS GTSYSLTISSMEAEDAATYYCQQWSSNPFTFGS GTKLEIK | 327 |
| SCT-Fa162 | QVQLQQPGTELVKPGASVKVSCKASGYTFISYW MHWVKQRPGQGLEWIGNIDPNYGGSNYNEKFKS KATLTVDKSSSTAYMQLSSLTSEDSAVYYCARW GWDGFAYWGQGTLVTVSA | 157 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQVTHV PYTFGGGTKLEIK | 192 |
| SCT-Fa163 | QVQLQQPGTELVKPGASVKVSCKASGYTFISYW MHWVKQRPGQGLEWIGNIDPNYGGSNYNEKFKS KATLTVDKSSSTAYMQLSSLTSEDSAVYYCARW GWDGFAYWGQGTLVTVSA | 157 | DIQMTQSPSSLSASLGERVSLTCRASQDIGSSL NWLQQEPDGTIKRLIYATSSLDSGVPKRFSGSR SGSDYSLTISSLESEDFVDYYCLQYASSPFTFG SGTKLEIK | 242 |
| SCT-Fa164 | QVQLQQPGTELVKPGASVKVSCKASGYTFISYW MHWVKQRPGQGLEWIGNINPNNGGTNYNEKFKS KATLTVDKSSSTAYMQLSSLTSEDSAVYYCARW GWDGFAYWGQGTLVTVSA | 158 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQVTHV PYTFGGGTKLEIK | 192 |
| SCT-Fa165 | QVQLQQPGTELVKPGASVKVSCKASGYTFVSYW IHWVKQRPGQGLEWIGNIDPNNGGSNYNERFRT KATLTVDKSSSTAYMQLSSLASEDSAVYYCARW GWDGFAYWGQGTLVTVSA | 159 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQLTHV PYTFGGGTKLEIK | 191 |
| SCT-Fa166 | QVQLQQPGTELVKPGASVKVSCKASGYTFVSYW MHWVKQRPGQGLEWIGNIDPNNGGSNYNERFRT KATLTVDKSSSTAYMQLSSLASEDSAVYYCARW GWDGFAYWGQGTLVTVSA | 160 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQLTHV PYTFGGGTKLEIK | 191 |
| SCT-Fa167 | QVQLQQPGTELVKPGASVKVSCKTSGYTFISYW MHWVKQRPGQGLEWIGNIDPNSGTTNYNEKFKT KATLTVDKSSSTAYMQLSSLTPEDSAVYYCARW GWDGFAYWGQGTLVTVSA | 161 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQVTHV PYTFGGGTKLEIK | 192 |
| SCT-Fa168 | QVQLQQPGTELVKPGASVKVSCKTSGYTFISYW MHWVKQRPGQGLEWIGNIDPNSGTTNYNEKFKT KATLTVDKSSSTAYMQLSSLTSEDSAVYYCARW GWDGFAYWGQGTLVTVSA | 162 | DAVMTQTPLSLPVSLGDQASISCRSSQSLENSN GNTYLNWYLQKPGQSPQLLIYRVSTRFSGVLDR FSGSGSGTDFTLKISRVEAEDLGVYFCLQVTHV PYTFGGGTKLEIK | 192 |
| SCT-Fa169 | QVQLQQPGTELVMPGASVNLSCKASGYTFTSYW MHWVKQRPGQGLEWIGEIDPSDSYANYNQNFKG KATLTVDKSSITAYMQLSSLTSEDSAVYYCARN YGSNYVGYFDYWGQGTTLTVSS | 163 | DIQMTQTTSSLSASLGDRVTISCRASQDISNYL NWYQQKPDGTVKLLIYYTSRLHSGVPSRFSGSG SGTDYSLTISNLDQEDIATYFCQQEKTLPWTFG GGTKLEIK | 248 |
| SCT-Fa170 | QVQLQQSGAELARPGASVKLSCKASGYTFRSYG LSWVKQRTGQGLEWIGEIYPRSGNTYYNEKFKD RVTLTADKSSSAAYMELRSLTSEDSAVYFCARS TAWAGGFDYWGQGTALTVSS | 164 | DIVMTQSPSSLTVTAGEKVTMSCKSSQSLFNSR TQKNSLTWYQQKPGQPPKLLIFWASTRESGVPD RFTGSASGTDFTLTLSGVQAEDLAIYYCQNDYT YPWTFGGGTKLEIK | 270 |
| SCT-Fa171 | QVQLQQSGAELARPGASVKLSCKASGYTFTNYG ISWVKQRTGQGLEWIGEIYPRSGNSYYNEKFKG KATLTADKSSSTAYMELRSLTSEDSAVYFCARS TAIVAGGFDYWGQGTTLTVSS | 165 | DIVMTQSPSSLTVTAGEKVTMSCKSSQSLLNSR IQKNSLTWYQQKPGQPPKLLIYWASTRESGVPD RFTASGSGTDFTLTISSVQAEDLAVYYCQNDYT YPWTFGGGTKLEIK | 273 |
| SCT-Fa172 | QVQLQQSGAELARPGASVKLSCKASGYTFTSYG ISWVKQRTGQGLEWIGEIYPRSGNTYYNEKFKG KATLTADKSSSTAYMELRSLTSEDSAVYFCARS TYGSSSYAMDYWGQGTSVTVSS | 166 | DVLMTQTPLSLPVSLGDQASISCRSSQSIVHSN GNTYLEWYLQKPGQSPKLLIYKVSNRFSGVPDR FSGSGSGTDFTLKISRVEAEDLGVYYCFQGSHV PYTFGGGTKLEIK | 281 |
| SCT-Fa173 | QVQLQQSGAELMRPGASVTLSCKASGYTFPDYE MYWVKQTPVHGLEWIGVIDPETGGTAYNQKFEV KAILTADKSSNTAYMELRSLTSEDSAVYYCTRW AYWGQGTLVTVSA | 167 | QIVLTQSPAIMSASLGERVTMTCTASSSVSSSY LHWYQQKPGSSPKLWVYSTSNLASGVPGRFSGS GSGTSYSLTISSMEAEDAATYYCHHYHRSPPTF GGGTKLEIK | 322 |
| SCT-Fa174 | QVQLQQSGAELVKPGASVKLSCKASGYSITGYY IHWVKQRPGQGLEWIGWIYPGSGSIKYNEKFKG KATLTADTSSSTADMQLSSLTSEDSAVYYCARE AWGNWYFDVWGTGTTVTVSS | 168 | DILLTQSPAILSVSPGERVSFSCRASQSIGTSI HWYQQRTNGSPRLLIKYASESISGIPSRFSGSG SGTDFTLSINSVESEDIADYYCQQSNSWPYTFG GGTKLEIK | 209 |
| SCT-Fa175 | QVQLQQSGAELVRPGASVTLSCKASGYTFSDYE MHWVKQTPVHGLEWIGVIDPETGDTAYNQKFKD KAVLTADKSSNTAYMELRSLTSEDSAVFFCTSN GYPEHWGQGTTLTVSS | 169 | QIVLTQSPAIMSASLGERVTMTCTASSGVSSTY LHWYQQRPGSSPKLWIYSTSTLASGVPARFSGS GSGTSYSLTISSMEAEDAATYYCHQYHRSPLTF GAGTKLELK | 321 |
| SCT-Fa176 | QVQLQQSGAELVRPGASVTLSCKASGYTFTDFE MHWVKQTPVHGLEWIGAIDPETGGTAYNQKFKG KAILTADRSSSTAYMELRSLTSEDSAVYYCTRG IYYGNYGSAMDYWGQGTSVTVSS | 170 | DVLMTQTPLSLPVSLGDQASISCRSSQSIVHSN GNTYLEWYLQKPGQSPKLLIYKVSNRFSGVPDR FSGSGSGTDFTLKISRVEAEDLGVYYCFQGSHA PLTFGAGTKLELK | 279 |
| SCT-Fa177 | QVQLQQSGAELVRPGASVTLSCKASGYTFTDYE MYWVKQTPVHGLEWIGVIDPETGGTAHDQKFKG KAILTADKSSSTAYMELRSLTSEDSAVYYCTRW AFWGQGTLVTVSA | 171 | DVVMTQTPLSLPVSLGDQASISCRSSQSLVHSN GNTYLHWYLQKPGQSPKLLIYKVSNRFSGVPDR FSGSGSGTDFTLRISRVEADDLGVYFCSQSTHV PYTFGGGTKLEIK | 303 |
| SCT-Fa178 | QVQLQQSGAELVRPGASVTLSCKASGYTFTDYE MYWVKQTPVHGLEWIGVIDPETGGTAHDQKFKG KAILTADKSSSTAYMELRSLTSEDSAVYYCTRW AFWGQGTLVTVSA | 171 | QIVLTQSPAIMSASLGERVTMTCTASSSVSSSY LHWYQQKPGSSPKLWVYSTSNLASGVPGRFSGS GSGTSYSLTISSMEAEDAATYYCHHYNRSPPTF GGGTKLEIK | 323 |
| SCT-Fa179 | QVQLQQSGAELVRPGTSVKVSCKASGYAFSNYL IEWVKQRPGQGLEWIGVINPGNGGTTYNEKFKD KATLTADRSSTTAYMQLSSLTSEDSAVFFCART AYGSPLDYWGQGTTLTVSS | 172 | DIKMTQSPSSMYASRGERVTITCKASQDINRYL SWLQQKPGKSPKTLIYRANRLVDGVPSRFSGSG SGQDYSLTISSLEYEDMGIYYCLQYDEFPYTFG GGTKLEIK | 207 |
| SCT-Fa180 | QVQLQQSGAELVRPGTSVKVSCKASGYAFTNYL IEWAKQRPGQGLEWIGVINPGSGATTYNEKFKG RATLTADMSSTTAYMQLSSLTSEDSAVFFCART AYGSPLDCWGQGTTLTVSS | 173 | DIKMTQSPSSMYASLGERVTITCKASQDINRYL SWLQQKAGKSPKTLIYRENRLVEGVPSRFSGSG SGQDYSLTISSLEYEDMGIYYCLQYDEFPYTFG GGTKLEIK | 200 |
| SCT-Fa181 | QVQLQQSGAELVRPGTSVKVSCKASGYAFTNYL IEWVKQRPGQGLEWIGVINPGSGATNYNEKFKG KATLTADKSSSTAYMQLRSLASEDSAVYFCARR TTWAPYAMDYWGQGTSVTVSS | 174 | DIQMTQTTSSLSASLGDRVTISCRASQDISNYL NWYQQKPDGTVELLIYYTSRLHSGVPSRFSGSG SGTDYSLTISNLEQEDIATYFCQQGNTLPRTFG GGTKLEIK | 247 |
| SCT-Fa182 | QVQLQQSGAELVRPGTSVKVSCKASGYAFTNYL IEWVKQRPGQGLEWIGVIS PGSGATTYNEKFKG KATLTADRSSTTAYMQLSSLTSEDSAVYFCARS AYGSPLDFWGQGTTLTVSS | 175 | DIKMTQSPSSMYASLGERVTITCKASQDINRYL SWFQQKPGKSPKTLIYRVNRLVDGVPLRFSGSG SGQDYSLTISSLDYEDMGIYYCQQYDEFPYTFG GGTKLEIK | 199 |
| SCT-Fa183 | QVQLQQSGAELVRPGTSVKVSCKASGYAFTNYL IEWVKQRPGQGLEWIGVIS PGSGATTYNEKFKG KATLTADRSSTTAYMQLSSLTSEDSAVYFCARS AYGSPLDFWGQGTTLTVSS | 175 | DILLTQSPDILSVSPGERVSFSCRASQNIGTSI HWYQQRTNGSPRLLIKYASESISGISSRFSGSG SGTDFTLSINSVESEDIADYYCQQSNNWPFTFG SGTKLEIK | 210 |
| SCT-Fa184 | QVQLQQSGAELVRPGTSVKVSCKASGYAFTNYL IEWVKQRPGQGLEWIGVISPGSGGTTYNEKFKG KATLTADKSSSTAYMQFSSLTSEDSAVYFCARS AYGHVFDYWGQGTTLTVSS | 176 | DIKMTQSPSSIYASLGERVTITCKASQDINSYL KWFQQKPGKSPKTLIYRANRLVDGVPSRFSGSG SGQDYSLTISSLEYEDMGIYYCLQYDEFPYTFG GGTKLEIK | 196 |
| SCT-Fa185 | QVQLQQSGAELVRPGTSVRVSCTASGYAFTNYL IEWVKQRPGQGLEWIGVISPGSGGTTYNEKFKG KATLTADKSSSTAYMQFSSLTSEDSAVYFCARS AYGHVFDYWGQGTTLTVSS | 177 | DIKMTQSPSSMYASLGERVTITCKASQDINSYL KWFQQKPGKSPKTLIYRANRLVDGVPSRFSGSG SGQDYSLTISSLEYEDMGIYYCLQYDEFPYTFG GGTKLEIK | 201 |
| SCT-Fa186 | QVQLQQSGPELARPGASVKLSCKASGYTFRSNG ISWVKQRTGQGLEWIGEIYPRSGNIYYNEKVKD KATLTADKSSNTAYMAVRSLTSEDSAVYFCARS TTWAGGFDFWGQGTVLTVSS | 178 | DIVMTQSPSSLTVTAGEKVTMSCKSSQSLLNSR TQKNSLTWYQQKPGQPPKLLIYWASTRESGVPD RFTGSGSGTDFTLTISSVQAEDLAIYYCQNDYT YPWTFGGGTELEIK | 274 |
| SCT-Fa187 | QVQLQQSGPELVKPGASVKISCKASGYSFTGYY IHWMKQRPGKGLEWIGWIYPGSGNTKYNEKFKG KATLTADTSSSTADMQLSSLTSEDSAVYYCARE AWGNWYFDVWGTGTTVTVSS | 179 | DILLTQSPAILSVSPGERVSFSCRASQSIGTSI HWYQQRTNGSPRLLIKYASESISGIPSRFSGSG SGTDFTLSINSVESEDIADYYCQQSNSWPYTFG GGTKLEIK | 209 |
| SCT-Fa188 | QVQLQQSGPELVKPGASVKISCKASGYSFTGYY IHWVKQRPGQGLEWIGWIYPGSGNTKYNEKFKG KATLTADTSSSTADMQLSSLTSEDSAVYYCARE AWGNWYFDVWGTGTTVTVSS | 180 | DILLTQSPAILSVSPGERVSFSCRASQSIGTSI HWYQQRTNGSPRLLIKYASESISGIPSRFSGSG SGTDFTLSINSVESEDIADYYCQQSNSWPYTFG GGTKLEIK | 209 |
| SCT-Fa189 | QVQLQQSGPELVKPGASVKISCKASGYSFTGYY IHYIHWVKQRPGQGLEWIGWIYPGSGNTKYNEK FKVKATLTADTSSSTADMQLSSLTSEDSAVYYC AREAWGNWYFDVWGTGTTVTVSS | 181 | DILLTQSPAILSVSPGERVSFSCRASQSIGTSI HWFQQRTNGSPRLLIKYASESISGIPSRFSGSG SGTDFTLSINSVESEDIADYYCQQSNSWPYTFG GGTKLEIK | 208 |
| SCT-Fa190 | QVQLRQPGAELVMPGASVKLSCKASGYTFNTYW MHWVKQRPGQGLEWIGEIDPSDSYTNYNQNFKG KATLTVDKSSSTAYLQLSSLTSEDSAVYYCARN YGSNYVGYFDYWGQGTTLTVSS | 182 | DIQMTQTTSSLSASLGDRVTISCRASQDISNYL NWYQQKPDGTVKLLIYYTSRLPSGVPSSFSGSG SGTDYSLTISNLEQEDIATYFCQQAKALPWTFG GGTKLEIK | 253 |
| SCT-Fa191 | QVQLRQSGAELARPGASVKLSCEASGYTFTSFG ITWVKQRPGQGLEWIGEIYPRSGNIYHNEKLKG KVTLTADKSSSTVYMELRSLTSEDSAVYFCARS TAVVAGGFDYWGQGTTLTVSS | 183 | DIVMTQSPSSLTVTTGEKVTVNCKSSQSLLNSR IQKNSLTWYQQKPGQPPKLLIYWSSTRESGVPD RFTGSGSGTDFTLTISSVQAEDLAIYYCQNDYN YPWTFGGGTKLEIK | 276 |
| SCT-Fa192 | QVQLRQSGAELARPGASVKLSCKASGYTFTNYG ITWVKQRTGQGLEWIGEIYPRSGNIYYNEKFKG KVTLTADKSSSTAYMELRSLTSEDSAVYFCARS TAVVAGGFDYWGQGTTLTVSS | 184 | DIVMTQSPSSLTVTTGEKVTVNCKSSQSLLNSR IQKNSLTWYQQKPGQPPKLLIFWASTRESGVPD RFTGSESGTDFTLTISSVQAEDLAIYYCQNDYN YPWTFGGGTKLEIK | 275 |
| SCT-Fa193 | QVQLRQSGAELARPGASVKLSCKASGYTFTSYG ITWVKQRTGQGLEWIGEIYPRSGNIYYNEKFKG KVTLTADKSSSTAYMELRSLTSEDSAVYFCARS TAVVAGGFDYWGQGTTLTVSS | 185 | DVVMTQSPSSLTVTTGERVTVNCKSSQSLLNSR IQKNSLTWYQQKPGQPPKLLIYWASTRESGVPD RFTGSGSGTDFTLTISSVQAEDLAIYYCQNDYN YPWTFGGGTKLEIK | 293 |
| SCT-Fa194 | QVQLRQSGAELVRPGTSVKLSCKASGYTLTDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 186 | DIQMNQSPSSLSASLGDTITITCHASQNINVWL SWYQQKPGNILKLLIYKASNLHTGVPSRFSGSG SGTGFTLTISSLQPEDIATYYCQQGQSYPLTFG SGTKLEIK | 226 |
| SCT-Fa195 | QVQLRQSGAELVRPGTSVKLSCKASGYTLTDYY INWVKQRPGQGLEWIARIYPGNGNTYYNEKFKG KATLTAEKSSSTVNMQVSSLTSEDSAVYFCARG GSSDRIYYYAMDYWGQGTSVTVSS | 186 | DIQVTQSSPFLSVSLGGRVIISCKASDHINNWL AWYQQKPGNAPRLLISGAINLETGVPSRFSGSG SGKDYTLSISSLQTEDVATYYCQQYWNTPYTFG GGTKLEIK | 258 |

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

### References Cited

### Other References

Anderson, A.C. (2014). Tim-3: an emerging target in the cancer immunotherapy landscape. Cancer Immunol. Res. 2, 393-398.

Fourcade, J., Sun, Z., Benallaoua, M., Guillaume, P., Luescher, I.F., Sander, C., Kirkwood, J.M., Kuchroo, V., and Zarour, H.M. (2010). Upregulation of Tim-3 and PD-1 expression is associated with tumor antigen-specific CD8+ T cell dysfunction in melanoma patients. J. Exp. Med. 207, 2175-2186.

Han, G., Chen, G., Shen, B., and Li, Y. (2013). Tim-3: an activation marker and activation limiter of innate immune cells. Front. Immunol. 4, 449.

Monney, L., Sabatos, C.A., Gaglia, J.L., Ryu, A., Waldner, H., Chernova, T., Manning, S., Greenfield, E.A., Coyle, A.J., Sobel, R.A., et al. (2002). Th1-specific cell surface protein Tim-3 regulates macrophage activation and severity of an autoimmune disease. Nature 415, 536-541.

Nakayama, M., Akiba, H., Takeda, K., Kojima, Y., Hashiguchi, M., Azuma, M., Yagita, H., and Okumura, K. (2009). Tim-3 mediates phagocytosis of apoptotic cells and cross-presentation. Blood 113, 3821-3830.

Riedl, S., Rinner, B., Asslaber, M., Schaider, H., Walzer, S., Novak, A., Lohner, K., and Zweytick, D. (2011). In search of a novel target - Phosphatidylserine exposed by non-apoptotic tumor cells and metastases of malignancies with poor treatment efficacy. Biochim. Biophys. Acta BBA - Biomembr. 1808, 2638-2645.

What is claimed is:

1. An antibody or an antigen binding fragment that binds human T-cell immunoglobulin and mucin-domain containing-3 (TIM-3) comprising SEQ ID NO:1, the antibody comprising:
a) a HCDR1 having the amino acid sequence of SEQ ID NO: 369, a HCDR2 having the amino acid sequence of SEQ ID NO:373, a HCDR3 having the amino acid sequence of SEQ ID NO:374, a LCDR1 having the amino acid sequence of SEQ ID NO:375, a LCDR2 having the amino acid sequence of SEQ ID NO:376, and a LCDR3 having the amino acid sequence of SEQ ID NO:377; or
b) a HCDR1 having the amino acid sequence of SEQ ID NO: 363, a HCDR2 having the amino acid sequence of SEQ ID NO:492, a HCDR3 having the amino acid sequence of SEQ ID NO:490, a LCDR1 having the amino acid sequence of SEQ ID NO:366, a LCDR2 having the amino acid sequence of SEQ ID NO:367, and a LCDR3 having the amino acid sequence of SEQ ID NO:493.

2. The antibody or the antigen binding fragment of claim 1, wherein the HCDR1 has the amino acid sequence of SEQ ID NO:369, the HCDR2 has the amino acid sequence of SEQ ID NO:373, the HCDR3 has the amino acid sequence of SEQ ID NO:374, the LCDR1 has the amino acid sequence of SEQ ID NO:375, the LCDR2 has the amino acid sequence of SEQ ID NO:376, and the LCDR3 has the amino acid sequence of SEQ ID NO:377.

3. The antibody or the antigen binding fragment of claim 1, wherein the HCDR1 has the amino acid sequence of SEQ ID NO:363, the HCDR2 has the amino acid sequence of SEQ ID NO:492, the HCDR3 has the amino acid sequence of SEQ ID NO:490, the LCDR1 has the amino acid sequence of SEQ ID NO:366, the LCDR2 has the amino acid sequence of SEQ ID NO:367, and the LCDR3 has the amino acid sequence of SEQ ID NO:493.

4. An antibody or the antigen binding fragment that binds human T-cell immunoglobulin and mucin-domain containing-3 (TIM-3) comprising SEQ ID NO:1, the antibody comprising:
a) a heavy chain variable domain (VH) having the amino acid sequence of SEQ ID NO: 12, and a light chain variable domain (VL) having the amino acid sequence of SEQ ID NO:241; or
b) a VH having the amino acid sequence of SEQ ID NO: 74, and a VL having the amino acid sequence of SEQ ID NO:326.

5. The antibody or the antigen binding fragment of claim 4, wherein the VH has the amino acid sequence of SEQ ID NO:12, and the VL has the amino acid sequence of SEQ ID NO:241.

6. The antibody or the antigen binding fragment of claim 4, wherein the VH has the amino acid sequence of SEQ ID NO:74, and the VL has the amino acid sequence of SEQ ID NO:326.

7. An antibody or the antigen binding fragment that binds human T-cell immunoglobulin and mucin-domain containing-3 (TIM-3) comprising SEQ ID NO:1, the antibody comprising:
a) a heavy chain comprising the amino acid sequence of SEQ ID NO: 12 and a light chain comprising the amino acid sequence of SEQ ID NO:241; or
b) a heavy chain comprising the amino acid sequence of SEQ ID NO: 74 and a light chain comprising the amino acid sequence of SEQ ID NO:326.

8. The antibody or the antigen binding fragment of claim 7, wherein the heavy chain comprises the amino acid sequence of SEQ ID NO: 12 and the light chain comprises the amino acid sequence of SEQ ID NO:241.

9. The antibody or the antigen binding fragment of claim 7, wherein the heavy chain comprises the amino acid sequence of SEQ ID NO: 74 and the light chain comprises the amino acid sequence of SEQ ID NO:326.

10. A pharmaceutical composition comprising the antibody or the antigen binding fragment of claim 1.

11. A kit comprising or the antigen binding fragment the antibody of claim 1.

12. The antibody or the antigen binding fragment of claim 1, comprising:
a) a heavy chain variable domain (VH) comprising the amino acid sequence of SEQ ID NO: 12 or an amino acid sequence that is at least 80% identical thereto, and a light chain variable domain (VL) having the amino acid sequence of SEQ ID NO:241 or an amino acid sequence that is at least 80% identical thereto; or
b) a VH having the amino acid sequence of SEQ ID NO: 74 or an amino acid sequence that is at least 80% identical thereto, and a VL having the amino acid sequence of SEQ ID NO:326 or an amino acid sequence that is at least 80% identical thereto.

13. The antibody of claim 1 which comprises a full-length immunoglobulin.

14. The antigen binding fragment of claim 11, wherein the fragment comprises any one of: a diabody, a single-chain antibody molecule, an Fab, an Fab′, an F(ab′)₂, an Fv, or a scFv.

15. A method of treating a cancer in a subject, comprising administering the antibody or the antigen binding fragment of claim 1 to the subject.

16. The method of claim 15, wherein the cancer expresses TIM-3, or the subject comprises a T cell expressing TIM-3, or both.

17. A method of treating a cancer in a subject, comprising administering the antibody or the antigen binding fragment of claim 12 to the subject.

18. A method of determining whether or not a subject is amenable to treatment with a TIM-3-specific anti-cancer therapeutic, comprising contacting an antibody or the antigen binding fragment of claim 1 with a sample and detecting binding of the antibody in the sample.

19. The method of claim 18, wherein the in contacting comprises a method selected from: an enzyme-linked immunosorbent assays (ELISA), a Western blot, an immunohistochemistry, an immunocytochemistry, a flow cytometry and fluorescence-activated cell sorting (FACS), an immunoprecipitation, or an enzyme-linked immunospot assays (ELISPOT).


SEQUENCE LISTING (722 sequences)

SEQ ID NO: 1 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 2 - protein, 197 aa, Artificial Sequence
  Human TIM-3 His tagged protein sequence
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGGGSGGGSHHHHHHHHHH

SEQ ID NO: 3 - protein, 416 aa, Artificial Sequence
  Human TIM-3 Human Fc tagged protein sequence
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 113 aa, Mus musculus
EFQLQQSGPELVKPGASVKISCKASGNSFTDYNLNWVKQSHGKSFEWIGVINPNYGTTSYNQKFKVKAALTVDQSSSTAYMQLNSLTSEDSAVYYCAREGNGWGQGTLVTVSA

SEQ ID NO: 5 - protein, 123 aa, Mus musculus
EFQLQQSGPELVKPGASVKISCKASGYSFTDYNMNWVKQSNGKSLEWIGVNNPKYGTTTYNQKFKGKATLTVDQSSSTAYMQLNSLTSEDSAVYYCARRGYYYGSSYGYFDVWGTGTTVTVSS

SEQ ID NO: 6 - protein, 117 aa, Mus musculus
EGKLEESGGGLVQPGGSMKLSCAASGFTFSDAWMDWVRQSPKKGLEWVAEIRNKANKHSKHYAESVKGRFTISRDDSKSSVYLQMNSLRAEDTGIYYCSYDYIFAYWGQGTLVTVSA

SEQ ID NO: 7 - protein, 117 aa, Mus musculus
EVHLQQSGAEFMKPGASVKLSCTASGFNIKGHYMHWVKQRTEQGLEWIGRIDPDDGATKFAPKFQGKATITADTSSNTVYLQLSSLTSEDTAVYYCTRSNPYYFDYWGQGTTLTVSS

SEQ ID NO: 8 - protein, 120 aa, Mus musculus
EVHLQQSVAEVVRPGASVNLSCTVSGFNNKNTYMYWVKQRPEQGLEWIGIIDPANGNVKYAPKFQGKATITADTSSNTAYLQLSSLTSEDTAIYYCARGSSRLYYALDYWGQGTSVTVSS

SEQ ID NO: 9 - protein, 125 aa, Mus musculus
EVHLVESGGGLVKPGGSLKLSCAASGFTFSDYGMHWVRQAPEKGLDWIAYISSGSISSDSSTIYYADTVKGRFTISRDNAKNTLFLQMTSLRSEDTAMYYCARHDWRGLYGMDYWGQGTSVTVSS

SEQ ID NO: 10 - protein, 117 aa, Mus musculus
EVKLVESEGGLVHPGSSMKLSCTASGFTFSDYYMAWVRQVPEKGLEWIANINFDGSGTYYLDSLKSRFIISRDNGKNILYLQMSGLKSEDTATYYCARLYDGYYAYWGQGTTLTVSS

SEQ ID NO: 11 - protein, 117 aa, Mus musculus
EVKLVESEGGLVQLGSSMKLSCTASGFTFSDYYMAWVRQVPEKGLEWIANINFDGSGTYYLDSLKSRFIISRDNGKNILYLQMSGLKSEDTATYYCARLYDGYYAYWGQGTTLTVSS

SEQ ID NO: 12 - protein, 123 aa, Mus musculus
EVKLVESEGGLVQPGSSMKISCTASGFTFSDYYMAWVRQVPEKGLEWVANINYDGSRTYYLDSLKSRFIISRDNAKNILYLQMSSLKSEDTATYYCARNIYYYGGSSYGMDYWGQGTSVTVSS

SEQ ID NO: 13 - protein, 117 aa, Mus musculus
EVKLVESEGGLVQPGSSMKLSCTASGFTFSDYYMAWVRQVPEKGLEWIANINFDGSGTYYLDSLKSRFIISRDNAKNILYLQMSSLKSEDTATYYCARLYDGSLDYWGQGTTLTVSS

SEQ ID NO: 14 - protein, 117 aa, Mus musculus
EVKLVESEGGLVQPGSSMKLSCTASGFTFSDYYMAWVRQVPEKGLEWVAHINFDGSGTYYLDSLKSRFIISRDNAKNILYLQLSSLKSEDTATYYCARLYDGSHDYWGQGTTLTVSS

SEQ ID NO: 15 - protein, 117 aa, Mus musculus
EVKLVESEGGLVQPGSSMKLSCTASGFTFSDYYMAWVRQVPEKGLEWVAHINFDGSGTYYLDSLKSRFIISRDNAKNILYLQLSSLKSEDTGTYYCARLYDGSHDYWGQGTTLTVSS

SEQ ID NO: 16 - protein, 117 aa, Mus musculus
EVKLVESEGGLVQPGSSMKLSCTASGFTFSDYYMAWVRQVPEKGLEWVAHINFDGSGTYYLDSLKSRFIISRDNEKSILYLQLSSLKSEDTATYYCARLYDGSHDYWGQGTTLTVSS

SEQ ID NO: 17 - protein, 117 aa, Mus musculus
EVKLVESEGGLVQPGSSMKLSCTASGFTFSDYYMAWVRQVPEKGLEWVAHINFDGSGTYYLDSLKSRFIISRDNTKNILYLQLSSLKSEDTATYYCARLYDGSHDYWGQGTTLTVSS

SEQ ID NO: 18 - protein, 117 aa, Mus musculus
EVKLVESEGGLVQPGSSMKLSCTASGFTFSDYYMAWVRQVPEKGLEWVANINFDGSGTYYLDSLKSRFIISRDNAKNILYLQMSSLKSEDTATYYCARLYDGSHDYWGQGTTLTVSS

SEQ ID NO: 19 - protein, 117 aa, Mus musculus
EVKLVESEGGLVQPGSSMKLSCTASGFTFSDYYMAWVRQVPEKGLEWVANINFDGSSSYYLDSLKSRFIISRDNTKNILYLQMSSLKSEDTATYYCARLYDGYYAYWGQGTTLTVSS

SEQ ID NO: 20 - protein, 117 aa, Mus musculus
EVKLVESEGGLVQPGSSMKLSCTASGFTFSDYYMSWVRQVPEKGLEWVANINFDGSGTYYLDSLKSRFIISRDNAKNILYLQMSSLRSEDTATYYCARLYDGSHDYWGQGSALTVSS

SEQ ID NO: 21 - protein, 113 aa, Mus musculus
EVQLHQSGPELVKPGASVKMSCKASGYTFTDYNMHWVKQSHGKSLEWIGYINPNNGGIIYNQNFKGKATLTVNKSSTTAYMELRSLTSEDSAVYYCVGSLDYWGQGTTLTVSS

SEQ ID NO: 22 - protein, 125 aa, Mus musculus
EVQLHQSGPVLVKPGASVKMSCKASEYTFADYYMNWVKQSHGKSLEWIGVINPYNGGATYNQKFKGKATLTVDKSSSTAYMEFSSLTSEDSAVYYCARDADFYGNSYVGYAMDYWGQGTSVTVSS

SEQ ID NO: 23 - protein, 120 aa, Mus musculus
EVQLQQPGAELVKPGASVKLSCKASGYTFTSYWIHWVKQRPGRGLEWIGRIDPNSGGTKYNEKFKSKATLTVDKPSSTAYMQLSSLTSEDSAVYYCTKDGNTPYYAMDYWGQGTSVTVSS

SEQ ID NO: 24 - protein, 117 aa, Mus musculus
EVQLQQSGAELVKPGASVKLSCTASDFNIKDYYLHWVKQRTEQGLEWIGRIDPDDGETKYGPKFQGKATITADTSSNTAFLQLSSLTSEDTAVYFCSRSNPYYFDYWGQGTTLTVSS

SEQ ID NO: 25 - protein, 117 aa, Mus musculus
EVQLQQSGAELVKPGASVKLSCTASDFNIKDYYMHWVKQSTEQGLEWIGRIDPEDDETKYAPKFQGKATITADTSSNTAYLQLSSLTSEDTAVYYCARSNPYYFDYWGQGTSLTVSS

SEQ ID NO: 26 - protein, 117 aa, Mus musculus
EVQLQQSGAELVKPGASVKLSCTASGFNIKDFYMHWVHQRTEKGLEWIGRIDPEDGKTKFAPKFQGKATITADTSSNTAYLQLSSLTSEDTAVYYCARSNPYFFDYWGQGTTLTVSS

SEQ ID NO: 27 - protein, 117 aa, Mus musculus
EVQLQQSGAELVKPGASVKLSCTASGFNIKDFYMHWVHQRTEKGLEWIGRIDPEDGKTKYAPKFQGKATITADTSSNTAYLQLSSLTSEDTAVYYCARSNPYFFDYWGQGTTLTVSS

SEQ ID NO: 28 - protein, 117 aa, Mus musculus
EVQLQQSGAELVKPGASVKLSCTASGFNIKDYYIHWVKQRTDQGLEWIGRIDPDNGETKYAPKFQGKATITADTSSNTAYLQLGSLTSEDTAVYYCARSSPYYFDYWGQGTTLTVSS

SEQ ID NO: 29 - protein, 117 aa, Mus musculus
EVQLQQSGAELVKPGASVKLSCTASGFNIKDYYMHWVKQRTDHGLEWIGRIDPDNGETKYAPKFQGKATITADTSSNTAYLQLGSLTSEDTAVYYCARSSPYYFDYWGQGTTLTVSS

SEQ ID NO: 30 - protein, 117 aa, Mus musculus
EVQLQQSGAELVKPGASVKLSCTASGFNIKDYYMHWVKQRTDQGLEWIGRIDPEDGKTKYAPKFQGKATITADTSSNTAFLQLTSLTSEDTAVYYCARSNPYYFDYWGQGTTLTVSS

SEQ ID NO: 31 - protein, 117 aa, Mus musculus
EVQLQQSGAELVKPGASVKLSCTASGFNIKDYYMHWVKQRTEQGLEWIGRIDPEDGETKYAPKFQGKATITADTSSNTASLQLSSLTSEDTAVYYCARSNPYFFDYWGQGTPLTVSS

SEQ ID NO: 32 - protein, 117 aa, Mus musculus
EVQLQQSGAELVKPGASVKLSCTASGFNIKDYYMHWVKQRTEQGLEWIGRIDPEDGETKYVPKFQGKATITADTSSNTAFLHLTSLTSEDTAVYYCARSNPYYFDYWGQGTTLTVSS

SEQ ID NO: 33 - protein, 117 aa, Mus musculus
EVQLQQSGAELVKPGASVKLSCTASGFNIKDYYMHWVRQRTEQGLEWIGRIDPEDGETKYAPKFQGKATITAATSSNTPYLQLSGLTSEDTAVYYCARSNPYYFDYWGQGTTLTVSS

SEQ ID NO: 34 - protein, 117 aa, Mus musculus
EVQLQQSGAELVKPGASVKLSCTASGFNIKDYYMHWVRQRTEQGLEWIGRIDPEDGETKYAPKFQGKATITAATSSNTPYLQLSGLTSEDTAVYYCGRSNPYYFDYWGQGTTLTVSS

SEQ ID NO: 35 - protein, 117 aa, Mus musculus
EVQLQQSGAELVKPGASVKLSCTDFGFNIKDYYMHWVKQRTEQGLEWIGRIDPEDGETKYVPKFQGKATITADTSSNTAFLHLTSLTSEDTAVYYCARSNPYYFDYWGQGTTLTVSS

SEQ ID NO: 36 - protein, 117 aa, Mus musculus
EVQLQQSGAELVKPGASVKLSCTPSDFNIKDYYIHWVRQRTEQGLEWIGRIDPEDGETKYAPKFQGKATITADTSSNTAYLQVSSLTSEDTAVYYCARSNPYYFDYWGQGTTLTVSS

SEQ ID NO: 37 - protein, 117 aa, Mus musculus
EVQLQQSGAELVKPGASVKLSCTVSGFNIKDYYMHWVKQRTEQGLEWIGRIDPDNDKIKYAPKFQGRATITADTSSNTVYLQLSSLTSEDTAVYYCARSNPYFFDYWGQGTTLTVSS

SEQ ID NO: 38 - protein, 117 aa, Mus musculus
EVQLQQSGAELVKPGASVKLSCTVSGFNIKDYYMHWVKQRTEQGLEWIGRIDPENDKIKYAPKFQGKATITADTSSNTVYLQLSSLTSEDTAVYYCARSNPYFFDYWGQGTTLTVSS

SEQ ID NO: 39 - protein, 117 aa, Mus musculus
EVQLQQSGAELVKPGASVKLSFTASDFNIKDYYIHWVKQRTEQGLEWIGRIDPEDGETRYAPKFQGKATITADTSSNTAYLQLSSVTSADTAVYFCTRSNPYYFDYWDQGTTLTVSS

SEQ ID NO: 40 - protein, 117 aa, Mus musculus
EVQLQQSGAELVKPGASVKLSFTASGFNIKDYYIHWVKQRTEQGLEWIGRIDPEDGETKYAPKFQGKATITADTSSNTAYLQLSSLTSEDTAVYFCTRSNPYYFDYWGQGTTLTVSS

SEQ ID NO: 41 - protein, 117 aa, Mus musculus
EVQLQQSGAELVKPGASVKLSYTSSGFNIKDYYIHWVKQRTDQGLEWIGRIDPDNGETKYAPKFQGKATIIADTSSNTAYLQLGSLTSEDTAVYYCARSSPYYFDYWGQGTTLTVSS

SEQ ID NO: 42 - protein, 117 aa, Mus musculus
EVQLQQSGAELVKPGASVKLSYTSSGFNIKDYYIHWVKQRTDQGLEWIGRIDPDNGETKYAPKFQGKATITADTSSNTAYLQLGSLTSEDTAVYYCARSSPYYFDYWGQGTTLTVSS

SEQ ID NO: 43 - protein, 117 aa, Mus musculus
EVQLQQSGAELVRPGASVKLSCTASGFNIKDYYMHWVKQRTEQGLEWIGRIDPDNDEIKYAPKFQGKATLTADTSSNTASLQLSGLTSEDTAVYYCARSSPYYFDYWGQGTTLTVSS

SEQ ID NO: 44 - protein, 117 aa, Mus musculus
EVQLQQSGAELVRPGASVKLSCTASGFNIKDYYMHWVKQRTEQGLEWIGRIDPDNDEIKYAPKFQGKATLTSDTSSNTASLQLSGLTSEDTAVYYCARSSPYYFDYWGQGTTLTVSS

SEQ ID NO: 45 - protein, 119 aa, Mus musculus
EVQLQQSGAEVVRPGASVKLSCTSSGFNIKDYYLHWVMQRTEHGLNWIGRIDPDDAETKYAPQFQGRATITADTSSNTAYLQLSSLTSEDTAVYYCTRDFGYTDYFDYWGQGTTLTVSS

SEQ ID NO: 46 - protein, 120 aa, Mus musculus
EVQLQQSGPELVKPGASVKISCKASGYTFTDYNMHWVKQSHGKSLAWIGYIYPYNGGTGYNQKFKNKATLTVDDSSSTAYMDLRSLTSDDSAVYYCAREDYGSREWFAYWGQGTLVTVSA

SEQ ID NO: 47 - protein, 113 aa, Mus musculus
EVQLQQSGPELVKPGASVKISCKASGYTFTDYYMNWVKQSHGKSLEWIGDINLNNGGTVYNQKFRGKATLTVDKSSSTAYMELRSLTSEDSAVYYCEGLGGYWGQGTTLTVSS

SEQ ID NO: 48 - protein, 113 aa, Mus musculus
EVQLQQSGPELVKPGASVKISCKASGYTLTDYYMNWVKQSHGESLEWIGDINPNNGDINYNQKFKGKATLTADKSSSTAYMELRSLTSEDSAVYYCEGLGGYWGQGTTLTVSS

SEQ ID NO: 49 - protein, 113 aa, Mus musculus
EVQLQQSGPELVKPGSSVKISCKASGYTLTDYYMNWVKQSHGESLEWIGDINPNNGDINYNQKFKGKATLTADKSSSTAYMELRSLTSEDSAVYYCEGLGGYWGQGTTLTVSS

SEQ ID NO: 50 - protein, 114 aa, Mus musculus
EVQLQQSGPMLVKPGASVKMSCKASGNTFTAFYMSWVKQSHGKSLEWIGVVNPYNGGAGYNQKFKGKATLTVDKSSSTAYMELNGLTSEDSAVYYCLLWEFAHWGQGTLVTVSA

SEQ ID NO: 51 - protein, 125 aa, Mus musculus
EVQLQQSGPVLVKPGASVKMSCKASEYTFTDYYMNWVKQSHGKTLEWIGVINPYNGGATYNQRFKDKATLTVDKSSSTAYMELNSLTSEDSAVYYCARDVDFYGNRYVGYAMDYWGQGTSVTVSS

SEQ ID NO: 52 - protein, 118 aa, Mus musculus
EVQLQQSGPVLVKPGASVKMSCKASGYSFTGYHMNWVKQSHGKSLEWIGIINPYNSASTYNQKFKGKATLTVDKSSSTAYMELNSLTSEDSVVYYCVRGAYGNPLDYWGQGTTLTVSS

SEQ ID NO: 53 - protein, 118 aa, Mus musculus
EVQLQQSGPVLVKPGASVKMSCKASGYSFTGYHMNWVKQSHGKSLEWIGIINPYNSATTYNQKFKGKATLTVDKSSSTAYMELNSLTSEDSVVYYCVRGAYGNPLDYWGQGTTLTVSS

SEQ ID NO: 54 - protein, 118 aa, Mus musculus
EVQLQQSGPVLVKPGASVKMSCKASGYSFTGYHMNWVKQSHGKSLEWIGIINPYNSATTYNQKFKGKATLTVDKSSSTAYMEVNSLTSEDSLVYYCVRGAYGNPLDYWGQGTTLTVSS

SEQ ID NO: 55 - protein, 118 aa, Mus musculus
EVQLQQSGPVLVKPGASVKMSCKASGYTFTDYHMNWVKQSHGKSLEWIGVISPYNGGTSQNQKFKGKATLTVDKSSSTAYMELNRLTSEDSAVYYCARSDYGSPLDYWGQGTTLTVSS

SEQ ID NO: 56 - protein, 125 aa, Mus musculus
EVQLQQSGPVLVKPGASVKMSCKASGYTFTDYYMNWVKQSHGKSLEWIGVINPYNGGATYNQNFKGKATLSVDKSSSTAYMELNSLTSEDSAVYYCARDVDFYDNAYVGYAMDYWGQGTSVTVSS

SEQ ID NO: 57 - protein, 122 aa, Mus musculus
EVQLQQSGPVLVKPGASVKMSCKASGYTFTDYYMNWVKQSHGKSLEWIGVINPYNGGTSYNQKFKGKATLTVDKSSSTAYMELNSLTSEDSAVYYCARGGDYDVNYSAMDYWGQGTSVTVSS

SEQ ID NO: 58 - protein, 125 aa, Mus musculus
EVQLQQSGPVLVKPGASVKMSCKVSEYTFTDYYMNWVKQSHGKSLEWIGVINPYNGGVTYNQKFKGKATLTVDKSSSTAYMELSSLTSEDSAVYYCARDADFHGNSYVGYAMDYWGQGTSVTVSS

SEQ ID NO: 59 - protein, 118 aa, Mus musculus
EVQLQQSGPVLVKPGASVKMSCQASGYSFTGYHMNWVKQSHGKSLEWIGIINPYNSGITYNQKFKGRATLTVDKSSDTAYMDLNSLTSEDSVVYYCVRGAYGNPLDYWGQGTTLTVSS

SEQ ID NO: 60 - protein, 125 aa, Mus musculus
EVQLQQSGPVLVKPGTSVKMSCKASGYTFTDYYLNWVKQSHGKSLEWIGVINPYNGGTTYNPKFKGKATLTVDKSSSTAYMELNSLTSEDSAVYYCARDVDYFDNKYVGYAMDYWGQGTSVTVSS

SEQ ID NO: 61 - protein, 118 aa, Mus musculus
EVQLQQSGPVLVRPGASVKMSCKASGYSFTGYHMNWVKQSHVKSLEWIGIINPYNSATTYNQNFKGKATLTVDKSSSTAYMELNSLTSEDSVVYYCVRGAYGNPLDYWGQGTTLTVSS

SEQ ID NO: 62 - protein, 117 aa, Mus musculus
EVQLQQSGTVLARPGASVKMSCKTPGYTFTRYWMHWVKQRPGQGLEWIGAIYPGNSDTNYNQKIKGKAKLTAVTTASTAYMELSSLTNEDSAVYYCTFYYGNYVNYWGQGTTLTVSS

SEQ ID NO: 63 - protein, 117 aa, Mus musculus
EVQLQQSGTVLARPGASVKMSCKTSGYTFIRYWMHWVKQRPGQGLEWIGAIYPGNSDTTYNQKFKGRAKLTAVTSASTAYMELSSLTNEDSAVYYCTFYYGNYVNFWGQGTTLTVSS

SEQ ID NO: 64 - protein, 117 aa, Mus musculus
EVQLQQSGTVLARPGASVKMSCKTSGYTFTRYWMHWVKQRPGQGLEWIGAIYPGNSDTNYNQKIKDKAKLTAVTTASTAYMELSSLTNEDSAVYYCTFFYGNYVNYWGQGTTLTVSS

SEQ ID NO: 65 - protein, 117 aa, Mus musculus
EVQLQQSGTVLARPGASVKMSCKTSGYTFTRYWMHWVKQRPGQGLEWIGAIYPGNSDTNYNQKIKGKAKLTAVTTASTAYMELISLTNEDSAVYYCTFYYGNYVNYWGQGTTLTVSS

SEQ ID NO: 66 - protein, 117 aa, Mus musculus
EVQLQQSGTVLARPGASVKMSCKTSGYTFTRYWMHWVKQRPGQGLEWIGAIYPGNSDTNYNQKIKGKAKLTAVTTASTAYMELSSLTNEDSAAYYCTFYYGNYVNYWGQGTTLTVSS

SEQ ID NO: 67 - protein, 117 aa, Mus musculus
EVQLQQSGTVLARPGASVKMSCKTSGYTFTRYWMHWVKQRPGQGLEWIGAIYPGNSDTNYNQKIKGKAKLTAVTTASTAYMELSSLTNEDSAVYYCTFYYGNYVNYWGQGTTLTVSS

SEQ ID NO: 68 - protein, 117 aa, Mus musculus
EVQLQQSGTVLARPGASVKMSCKTSGYTFTRYWMHWVKQRPGQGLEWIGAIYPGNSDTNYNQKIQDKAKLTAVTTASTAYMELSSLTNEDSAVYYCTFFYGNYVNYWGQGTTLTVSS

SEQ ID NO: 69 - protein, 117 aa, Mus musculus
EVQLQQSGTVLARPGASVKMSFKTSGYTFTRYWMHWVKQRPGQGLEWIGAIYPGNSDTNYNQKIKGKAKLTAVTTASTAYMELSSLTNDDSAVYYCTFYYGNYVNYWGQGTTLTVSS

SEQ ID NO: 70 - protein, 117 aa, Mus musculus
EVQLQQSVAELVRPGASVKLSCTASGFNIKDFYMHWVHQRTEKGLEWIGRIDPEDGKTKYAPKFQGKATITADTSSNTAYLQLSSLTSEDTAVYYCARSNPYFFDYWGQGTTLTVSS

SEQ ID NO: 71 - protein, 124 aa, Mus musculus
EVQLVESGGDLVKPGGSLKLSCAASGFTFSDYGMSWVRQTPDKRLEWVATISSGGSYTYYADSVKGRFTISRDNAKNTLYLQMSSLKSEDTAMYFCTRHEITTVLSTDYAMDYWGQGTSVTVSS

SEQ ID NO: 72 - protein, 125 aa, Mus musculus
EVQLVESGGGLVRPGGSLKLSCAASGFTFSDYGMHWVRQAPEKGLDWIAYISSGSISSGSSTIYYADTVKGRFTISRDNAKNTLFLQMTSLRSEDTAMYYCARHDWRGLYAMDYWGQGTSVTVSS

SEQ ID NO: 73 - protein, 125 aa, Mus musculus
EVQLVESGGGLVRPGGSLKLSCVVSGFTFSDYGMHWVPQAPEKGLDWIAYISGGSISSGSSTIYYADTVKGRFTISRDNAKNTLFLQMTSLRSEDTAMYYCARHDWRGLYAMDYWGQGTSVTVSS

SEQ ID NO: 74 - protein, 125 aa, Mus musculus
EVQLVESGGGLVRPGGSLKLSCVVSGFTFSDYGMHWVRQAPEKGLDWIAYISGGSISSGSSTIYYADTVKGRFTISRDNAKNTLFLQMTSLRSEDTAMYYCARHDWRGLYAMDYWGQGTSVTVSS

SEQ ID NO: 75 - protein, 114 aa, Mus musculus
EVQVLETGGGLVQPGGSRGLSCEGSGFTFSGFWMSWVRQTPGKTLEWIGDINSDGSTIHYGPSMKDRFTIFRDNDKNTLYLQMNNVRSEDTAKYFCMSLLLNVWGTGTTVTVSS

SEQ ID NO: 76 - protein, 122 aa, Mus musculus
EVRLVESEGGLVQPGSSMKLSCTASGFTFSDYYMAWVRQVPEKGLEWVANINYDGTNTYYLDSLKSRFIISRDNAKNILYLQMSGLKSEDTATYYCGRGIYYYGRSHAMDYWGQGTSVTVSS

SEQ ID NO: 77 - protein, 119 aa, Mus musculus
QGQLQQPGAEVVKPGAAVKLSCKASGYTFTSYWMQWIKQRPGQGLDWIGEIDPSDNYTDYNQNFKGKATLTVDTSSNTAYLQLSSLTSEDSAVYYCAREGAFSSPFAYWGPGTLVTVSS

SEQ ID NO: 78 - protein, 119 aa, Mus musculus
QGQLQQPGAEVVKPGAAVKLSCKASGYTFTSYWMQWIKQRPGQGLEWIGEIDPSDNYTDYHQNFKGKATLTVDTSSSTAYLHLSSLTSEDSAVYYCAREGAFSSPFAYWGPGTLVTVSS

SEQ ID NO: 79 - protein, 119 aa, Mus musculus
QGQLQQPGAEVVKPGAAVKLSCKASGYTFTSYWMQWIKQRPGQGLEWIGEIDPSDNYTDYNQHFKGKATLTVDTSSSTAYLQLSSLTSEDSAVYYCAREGAFSSPFAYWGPGTLVTVSS

SEQ ID NO: 80 - protein, 119 aa, Mus musculus
QGQLQQPGAEVVKPGAAVKLSCKASGYTFTSYWMQWIKQRPGQGLEWIGEIDPSDNYTDYNQNFKGKATLTVDTSSSTAYLQLSSLTSEDSAVYYCAKEGAFSSPFAYWGPGTLVTVSS

SEQ ID NO: 81 - protein, 119 aa, Mus musculus
QGQLQQPGAEVVKPGAAVKLSCKASGYTFTSYWMQWIKQRPGQGLEWIGEIDPSDNYTDYNQNFKGKATLTVDTSSSTAYLQLSSLTSEDSAVYYCAREGAFSSPFAYWGPGTLVTVSS

SEQ ID NO: 82 - protein, 119 aa, Mus musculus
QGQLQQPGAEVVKPGAAVKLSCKASGYTFTSYWMQWIKQRPGQGLEWIGEIDPSDNYTDYNQNFKGKATLTVDTSSSTAYLQLSSLTSKDSAVYYCAREGAFSSPFAYWGPGTLVTVSS

SEQ ID NO: 83 - protein, 119 aa, Mus musculus
QGQLQQPGAEVVKPGAAVNLSCKASGYTFTSYWMQWIKQRPGQGLEWIGEIDPSDNYTDYNQNFKGKATLTVDTSSSTAYLQLSSLTSEDSAVYYCAREGAFSSPFAYWGPGTLVTVSS

SEQ ID NO: 84 - protein, 118 aa, Mus musculus
QIQLQQSGPELVKPGASVKISCKASGYTFTDYYINWVKQRPGQGLEWIGWIYPGSDNTKYNEKFKGKSTLTVDTSSSTAYMQLSSLTSEDSAVYFCARSGNYYAMDYWGQGTSVTVSS

SEQ ID NO: 85 - protein, 117 aa, Mus musculus
QIQLQQSGPELVKPGASVKISCKASGYTFTDYYINWVKQRPGQGLEWIGWIYPGSGNTKYNEKFKGKATLTVDTSSNTAYMQLSSLTSEDSAVYFCARAYDYGFPYWGQGTLVTVSA

SEQ ID NO: 86 - protein, 117 aa, Mus musculus
QIQLQQSGPELVKPGASVKISCKASGYTFTDYYINWVKQRPGQGLEWIGWIYPGSGNTKYNEKFKGKATLTVDTSSSTAYMHLSSLTSEDSAVYFCARAYDYGFAYWGQGTLVTVSA

SEQ ID NO: 87 - protein, 117 aa, Mus musculus
QIQLQQSGPELVKPGASVKISCKASGYTFTDYYINWVKQRPGQGLEWIGWIYPGSGNTKYNEKFKGKATLTVDTSSSTAYMQLSSLTSEDSAVYFCARAYDYGFAYWGQGTLVTVSA

SEQ ID NO: 88 - protein, 118 aa, Mus musculus
QIQLQQSGPELVKPGASVKISCKASGYTFTDYYINWVKQRPGQGLEWIGWIYPGSGNTKYNEKFKGKSTLTVDTSSSTAYMQLSSLTSEDSAVYFCARSGNYYAMDYWGQGTSVTVSS

SEQ ID NO: 89 - protein, 116 aa, Mus musculus
QIQLVQSGPEMKKPGETVKISCKASGYIFRTYGMSWVKQAPGKGLEWMGWINTYSGEPTYADDFKGRFAFSLETSANTAYLQINNLKNEDTATFFCARAQHYFDWWGQGTTLTVSS

SEQ ID NO: 90 - protein, 116 aa, Mus musculus
QIQLVQSGPEMKKPGETVKISCKASGYTFRTYGMSWVKQAPGKGLEWMGWINTYSGEPTYADDFKGRFAFSLETSANTAYLQINNLKNEDTATFFCARAQHYFDWWGQGTTLTVSS

SEQ ID NO: 91 - protein, 123 aa, Mus musculus
QVHLKQSGAELVRPGTSVKLSCKASGYTLTDYYINWVKQRPGQGLEWIARIYPGNGNTYYNEKFKGKATLTAEKSSSTVNMQVSSLTSEDSAVYFCARGGSSDRIYYYAMDYWGQGTSVTVSS

SEQ ID NO: 92 - protein, 119 aa, Mus musculus
QVLLQQPGAELVRPGTSVKLSCKASGYTFTNFWMHWVKQRPGQGLEWIGVIDPSDISTHYNQNFKGKAILTVDTSSSSAYMQLSSLTSEDSAVYYCARNDYDGGYFDYWGQGTTLTVSS

SEQ ID NO: 93 - protein, 115 aa, Mus musculus
QVQLKESGPGLVAPSQSLSITCTVSGFSLTSSVISWVRQPPGKGLEWLGIIWAGGDTNYNSALKSRLSISKDNSKSQVFLKMNSLQTDDTARYYCARNLNAMDYWGQGTSVTVSS

SEQ ID NO: 94 - protein, 117 aa, Mus musculus
QVQLKQSGAELVRPGTSIKLSCKASGYTFTDFYINWVKQRPGQGLEWIARIYPGGANTYYNDNFKGKATLTAEKSSITAYMQLSSLTSEDSAVYFCARGANWAFASWGQGTLVTVSA

SEQ ID NO: 95 - protein, 123 aa, Mus musculus
QVQLKQSGAELVRPGTSVKLSCKASGYILTDYYINWVKQRPGQGLEWIARIYPGNGNTYYNEKFKGKATLTAEKSSSTVNMQVSSLTSEDSAVYFCARGGSSDRIYYYAMDHWGQGTSVTVSS

SEQ ID NO: 96 - protein, 123 aa, Mus musculus
QVQLKQSGAELVRPGTSVKLSCKASGYILTDYYINWVKQRPGQGLEWIARIYPGNGNTYYNEKFKGKATLTAEKSSSTVNMQVSSLTSEDSAVYFCARGGSSDRIYYYAMDYWGQGTSVTVSS

SEQ ID NO: 97 - protein, 123 aa, Mus musculus
QVQLKQSGAELVRPGTSVKLSCKASGYTLIDYYINWVKQRPGQGLEWIARIYPGNGNTYYNEKFKGKATLTAEKSSSTVNMQVSSLTSEDSAVYFCARGGSSDRIYYFAMDYWGQGTSVTVSS

SEQ ID NO: 98 - protein, 123 aa, Mus musculus
QVQLKQSGAELVRPGTSVKLSCKASGYTLIDYYINWVKQRPGQGLEWIARIYPGNGNTYYNEKFKGKATLTAEKSSSTVNMQVSSLTSEDSAVYFCARGGSSDRIYYYAMDYWGQGTSVTVSS

SEQ ID NO: 99 - protein, 123 aa, Mus musculus
QVQLKQSGAELVRPGTSVKLSCKASGYTLNDYYINWVKQRPGQGLEWIARIYPGNNNTYYNEKFKGKATLTAEKSSNTAYMQVSSLTSEDSAVYFCARGGSSVRVYYYAMDYWGQGTSVTVSS

SEQ ID NO: 100 - protein, 123 aa, Mus musculus
QVQLKQSGAELVRPGTSVKLSCKASGYTLTDYYINWVKQRPGQGLEWIARIYPGNGNTLYNAKFEGKATLTAERSSSTAYMQVSSLTSEDSAVYFCARGGSSVRVYYYAMDFWGQGTSVTVSS

SEQ ID NO: 101 - protein, 123 aa, Mus musculus
QVQLKQSGAELVRPGTSVKLSCKASGYTLTDYYINWVKQRPGQGLEWIARIYPGNGNTYYNEKFKCKATLTAEKSSSTVNMQVTSLTSEDSAIYFCARGGSSDRIYYYAMDYWGQGTSVTVSS

SEQ ID NO: 102 - protein, 123 aa, Mus musculus
QVQLKQSGAELVRPGTSVKLSCKASGYTLTDYYINWVKQRPGQGLEWIARIYPGNGNTYYNEKFKGKATLTAEKSSNTVNMQVSSLTSEDSAVYFCARGGSSDRIYYYAMDYWGQGTSVTVSS

SEQ ID NO: 103 - protein, 123 aa, Mus musculus
QVQLKQSGAELVRPGTSVKLSCKASGYTLTDYYINWVKQRPGQGLEWIARIYPGNGNTYYNEKFKGKATLTAEKSSSTAYMQVSSLTSEDSAVYFCARGGSSVRVYYYAMDYWGQGTSVTVSS

SEQ ID NO: 104 - protein, 123 aa, Mus musculus
QVQLKQSGAELVRPGTSVKLSCKASGYTLTDYYINWVKQRPGQGLEWIARIYPGNGNTYYNEKFKGKATLTAEKSSSTVNMQVSSLTSEDSAVYFCARGGSSDRIYYYAMDYWGQGTSVTVSS

SEQ ID NO: 105 - protein, 123 aa, Mus musculus
QVQLKQSGAELVRPGTSVKLSCKASGYTLTDYYINWVKQRPGQGLEWIARIYPGNGNTYYNEKFKGKATLTAEKSSSTVNMQVTSLTSEDSAIYFCARGGSSDRIYYYAMDYWGQGTSVTVSS

SEQ ID NO: 106 - protein, 123 aa, Mus musculus
QVQLKQSGAELVRPGTSVKLSCKASGYTLTDYYINWVKQRPGQGLEWIARIYPGNGNTYYNEKFKGKATLTAEKSSSTVNMQVTSLTSEDSAIYFCARGGSSDRIYYYAMDYWGQVTSVTVSS

SEQ ID NO: 107 - protein, 123 aa, Mus musculus
QVQLKQSGAELVRPGTSVRLSCKASGYTLTDYYINWVKQRPGQGLEWIARIYPGNGNTYYNEKFKGKATLTAEKSSSTVNMQVTSLTSEDSAVYFCARGGSSDRIYYYAMDYWGQGTSVTVSS

SEQ ID NO: 108 - protein, 115 aa, Mus musculus
QVQLKQSGPGLVQPSQSLSITCTVSGFSLISYGVHWVRQSPGKGLEWLGIIWSGGNTDYNAAFISRLSISKDNSKSQVFFKLNSLQADDTAIYYCARTANALDYWGQGTSVTVSS

SEQ ID NO: 109 - protein, 119 aa, Mus musculus
QVQLQQPGADVVKPGASVKLSCKASGYTFTNYWIQWIKQRPGQGLEWIGEIDPSDNYTDYNQNFKGKATMTVDTSSSTAYLQLSSLTSEDSAVYFCAGEGAFSSPFAYWGPGTLVTVSS

SEQ ID NO: 110 - protein, 119 aa, Mus musculus
QVQLQQPGAEFVKPGASVKLSCEASGNTFTSYWIQWVKQRPGQGLEWIGEIDPSDSYIDYNQKFKDKATLTVDTSSSTAYMHLSSLTSEDSAVYYCAREGAYSSPFAYWGQGTLVTVSA

SEQ ID NO: 111 - protein, 116 aa, Mus musculus
QVQLQQPGAEFVKPGASVKLSCKASGYTFTSYWMHWMKQRPGRGLEWIGRIDPDSGGTKYNEKFKNKATLTVDKPSSTAYMQLSSLTSEESAVYYCARYYWFFDIWGTGTTVTVSS

SEQ ID NO: 112 - protein, 116 aa, Mus musculus
QVQLQQPGAELVKPGASVKLSCKASCYTFISYWMHWMKQRPGRGLEWIGRIDPDSGGTKYNEKFKSKATLTVDKPSSTAYMQLSSLTSEDSAVYYCARYYWFFDIWGTGTTVTVSS

SEQ ID NO: 113 - protein, 120 aa, Mus musculus
QVQLQQPGAELVKPGASVKLSCKASGYSFISSWMEWVKQRPGQGLEWIGRIHPSDSDTNYNQKFKAKATLTVDKSSSTAYMQLSSLTSEDSAVYYCAIIHRGFYFAMDYWGQGTSVTVSS

SEQ ID NO: 114 - protein, 116 aa, Mus musculus
QVQLQQPGAELVKPGASVKLSCKASGYTFISYWMHWMKQRPGRGLEWIGRIDPDSGGTKYNEKFKSKATLTVDKPSSTAYMQLSSLTSEDSAVYYCARYYWFFDIWGTGTTVTVSS

SEQ ID NO: 115 - protein, 117 aa, Mus musculus
QVQLQQPGAELVKPGASVKLSCKASGYTFISYWMHWVKQRPGRGLEWIGRIDPNSGDTKYNEKFKSKATLTVDKPSSTAYMQLSSLISEDSAVYYCARWGWDAFAYWGQGTLVTVSA

SEQ ID NO: 116 - protein, 120 aa, Mus musculus
QVQLQQPGAELVKPGASVKVSCKASGYSFISSWMEWVKQRPGQGLEWIGRIHPSDSDTNYNQKFKGKATLTVDKSSSTAYMQLSSLTSEDSAVYYCAIIHRGFYFAMDYWGQGTSVTVSS

SEQ ID NO: 117 - protein, 120 aa, Mus musculus
QVQLQQPGAELVKPGASVKVSCKASGYSFISYWMYWVKQRPGQGLEWIGRIHPSDSDTNYNQKYKGKATLTVDRSSSTAYMQLSSLTSEDSAVYYCTISHYSGSSPETYWGQGTTLTVSS

SEQ ID NO: 118 - protein, 120 aa, Mus musculus
QVQLQQPGAELVKPGASVKVSCKASGYTFISHWMYWVRQRPGQGLEWIGRIHPSDSDTDYSQEFKGKATLTVDKSSSTAHMQLSSLTSEDSAVYYCAIWSYYYGSRLDYWGQGTTLTVSS

SEQ ID NO: 119 - protein, 121 aa, Mus musculus
QVQLQQPGAELVMPGASVKLSCEASGYTFTTFWIHWVKQRPGQGLEWIGEIDPSDSYTNYNQNFKGKATLTVDKSSSTAYLQLSSLTSEDSAVYYCARNYGSNYVGYFDYWGQGTTLTVSS

SEQ ID NO: 120 - protein, 121 aa, Mus musculus
QVQLQQPGAELVMPGASVKLSCKASGYTFNTYWMHWVKQRPGQGLEWIGEIDPSDSYTNYNQNFNGKATLTVDKSSSTAYLQLSSLTSEDSAVYYCARNYGSNYVGYFDYWGQGTTLTVSS

SEQ ID NO: 121 - protein, 121 aa, Mus musculus
QVQLQQPGAELVMPGASVKLSCKASGYTFSTYWMHWVKQRPGQGLEWIGEIDPSDSYTNYNQNFKGKATLTVDKSSSTAYMQLSSLTSEDSAVYYCARNYGSNYVGYFDHWGRGTTLTVSS

SEQ ID NO: 122 - protein, 121 aa, Mus musculus
QVQLQQPGAELVMPGASVKLSCKASGYTFTSYWMHWVKQRPGQGLEWIGEIDPSDNYTNYNQKFKGKATLTVDGSSSTAYMQLSSLTSEDSAVYYCARNYGSNYVGYFDYWGQGTTLTVSS

SEQ ID NO: 123 - protein, 121 aa, Mus musculus
QVQLQQPGAELVMPGASVKLSCKASGYTFTSYWMHWVKQRPGQGLEWIGEIDPSDSYSNYNQKFKGKATLTVDKSSSTAYMQLISLTSEDSAVYYCARNYGSNYVGYFDYWGQGTTLTVSS

SEQ ID NO: 124 - protein, 121 aa, Mus musculus
QVQLQQPGAELVMPGASVKLSCKASGYTFTTYWMHWVKQRPGQGLEWIGEIDPSDGYSNYNQNFKGKATLTVDKSSSTAYLQLSSLTSEDSAVYYCARNYGSNYVGYFDYWGQGTTLTVSS

SEQ ID NO: 125 - protein, 121 aa, Mus musculus
QVQLQQPGAELVMPGASVKLSCKASGYTFTTYWMHWVKQRPGQGLEWIGEIDPSDSYSNYNQKFKGKATLTVDKSSSTAYMQLSSLTSEDSAVYYCARNYGSNYVGYFDYWGQGTTLTVSS

SEQ ID NO: 126 - protein, 121 aa, Mus musculus
QVQLQQPGAELVMPGASVKLSCKASGYTFTTYWMHWVKQRPGQGLEWIGEIDPSDSYTNYNQKFKGKATLTVDKSSSTAYMQLSSLTSEDSAVYYCARNYGSNYVGYFDYWGQGTTLTVSS

SEQ ID NO: 127 - protein, 121 aa, Mus musculus
QVQLQQPGAELVMPGASVRLSCKASGYTFTTYWMHWVRQRPGQGLEWIGEIDPSDSYTNYNQNFKGKATLTVDKSSSTAYLQLSSLTSEDSAVYYCARNYGSNYVGYFDHWGQGTTLTVSS

SEQ ID NO: 128 - protein, 121 aa, Mus musculus
QVQLQQPGAELVMPGASVRLSCKTSGYTFSTYWMHWVKQRPGQGLEWIGEIDPSDSYTNYNQNFKGKATLTVDKSSSTAYMKLSSLTSEDSAVYYCARNYGSNYVGYFDYWGQGTTLTVSS

SEQ ID NO: 129 - protein, 121 aa, Mus musculus
QVQLQQPGAELVMPRASVKLSCKASGYTFTSYWMHWVKQRPGQGLEWIGEIDPSDGYTNYNHKFKGKATLTVDKSSSTAYMQLSSLTSEDSAVYYCARNYGSNYVGYFDYWGQGTTLTVSS

SEQ ID NO: 130 - protein, 121 aa, Mus musculus
QVQLQQPGAELVMSGASVKLSCKASGYTFTTYWMHWVRQRPGQGLEWIGEIDPSDSYSNNNQNFKAKATLTVDKSSSTAYMQLGSLTSEDSAVYYCARNYGSNYVGYFDYWGQGTTLTVSS

SEQ ID NO: 131 - protein, 113 aa, Mus musculus
QVQLQQPGAELVRPGSSVKLSCKASGYNFTNYWIDWVKQRPGQGLAWIGDIYPSDNKTHYNQNFKGKATLTVDKSSNTAYMQLSSLTSEDSAVYYCAASMDYWGQGTSVTVSS

SEQ ID NO: 132 - protein, 113 aa, Mus musculus
QVQLQQPGAELVRPGSSVKLSCKASGYNFTSYWIDWVKQRPGQGLEWIGDIYPSDSKTHYNQNFKGKATLTVDKSSNTAYMQLSNLTSEDSAVYYCATSMDYWGQGTSVTVSS

SEQ ID NO: 133 - protein, 119 aa, Mus musculus
QVQLQQPGAELVRPGTSVKLSCKASGYTFINYWMHWVKQRPGQGLEWIGVIDPSHTSTSYNQKFKGKATLTVDTSSTTTSMQLTSLTSEDSAVYYCARNDYDGGYFDYWGQGTTLTVSS

SEQ ID NO: 134 - protein, 119 aa, Mus musculus
QVQLQQPGAELVRPGTSVKLSCKASGYTFSRYWIHWVKQRPGQGLEWIGVIDPSDNNTHYNQKFKGKATLTVDASSSTAYMQLSSLTSEDSAVFFCARNDYDGGYFDYWGQGTTLTVSS

SEQ ID NO: 135 - protein, 119 aa, Mus musculus
QVQLQQPGAELVRPGTSVKLSCKASGYTFTRYWMHWVRQRPGQGLEWIGVIDPSSSSTDYNQKFKGKATLTVDTSSSTAYMQLSSLTSEDSAVYYCARNDYDGGYFDYWGQGTTLTVSS

SEQ ID NO: 136 - protein, 119 aa, Mus musculus
QVQLQQPGAELVRPGTSVKLSCKASGYTFTSYWMHWVNQRPGQGLEWIGVIDPSDSSTDYNQKFKGKATLTVDTSSSTAYMQLSSLTSEDSAVYYCARNDYDGGYFDYWGQGTTLTVSS

SEQ ID NO: 137 - protein, 119 aa, Mus musculus
QVQLQQPGAELVRPGTSVKLSCKASGYTFTSYWMHWVRQRPGQGLEWIGVIDPSSSSTDYNQKFKGKATLTVDTSSSTAYMQLSSLTSEDSAVYYCARNDYDGGYFDYWGQGTTLTVSS

SEQ ID NO: 138 - protein, 119 aa, Mus musculus
QVQLQQPGAELVRPGTSVNLSCKASGYTFTSYWMHWVKQRPGQGLEWLGVIDPSDSNTNYNQKFKGKATLTVDTSSSTAYMQLSSLTSEDSAVYYCSRNDYDGGYFDYWGQGTTLTVSS

SEQ ID NO: 139 - protein, 119 aa, Mus musculus
QVQLQQPGAELVRPGTSVRLSCKASGYSFTIYWMHWVKQRPGQGLEWIGVIDPSTSNTDYNQKFKGKATLTVDTSSSTAYMQLRSLSSEDSAVYYCARNDYDGGYFDYWGQGTTLTVSS

SEQ ID NO: 140 - protein, 119 aa, Mus musculus
QVQLQQPGAELVRSGTSVKLSCKASGYTFISYWMHWVKQRPGQGLEWIGVIDPSDTNTNYNQKFRGKATLTVDTSSSTAYMQLSSLTSEDSAVYFCARNDYDGGYFDYWGQGTTLTVSS

SEQ ID NO: 141 - protein, 121 aa, Mus musculus
QVQLQQPGAELVTPGASVKLSCKASGYTFTTYWMHWVKQRPGQGLEWIGEIDPSDSYRNYNQKFKGKATLTVDKSSSTAYMQLSSLTSEDSAVYYCARNYGSNYVGYFDYWGQGTALTVSS

SEQ ID NO: 142 - protein, 119 aa, Mus musculus
QVQLQQPGAEVVKPGASVKLSCKASGYTFTNYWMQWVRQRPGQGLEWIGEIDPSDNYTDYNQKFKGKATLTVDASSSTAYMQLSSLASEDSAVYFCAREGAFSSPFAFWGPGTLVTVSA

SEQ ID NO: 143 - protein, 119 aa, Mus musculus
QVQLQQPGAEVVKPGASVKLSCKASGYTFTNYWVQWVKQRPGQGLEWIGEIDPSDNYTDYNQNFKGKATLTVDTSSSTAYLHLSSLTSEDSAVYYCAREGAFSSPFAYWGPGTLVTVSS

SEQ ID NO: 144 - protein, 119 aa, Mus musculus
QVQLQQPGAEVVKPGASVKLSCKASGYTFTSYWMQWIKQRPGQGLEWIGEIDPSDNYTDYNQNFKGKATLTVDTSSSTAYLQLSSLASEDSAVYCCAREGAFSSPFAYWGPGTLVTVSS

SEQ ID NO: 145 - protein, 119 aa, Mus musculus
QVQLQQPGAEVVKPGASVKLSCKASGYTFTSYWMQWVKQRPGQGLEWIGEIDPSDNYTDYNQNFKGKATLTVDTSSSTAYMQLSSLTSEDSAVYYCAKEGAYSSPFAYWGPGTLVTVSA

SEQ ID NO: 146 - protein, 119 aa, Mus musculus
QVQLQQPGAEVVKPGASVKLSCKASGYTFTTYWMQWVKQRPGQGLEWIGEIDPSDNYTDYNQNFKGKATLTVDTSSNTAFMHLSSLTSEDSAVYYCVREGAFSSPFAYWGPGTLVTVSA

SEQ ID NO: 147 - protein, 120 aa, Mus musculus
QVQLQQPGAEVVKPGASVKVSCKASGYSFISHWMYWVRQRPGQGLEWIGRIHPSDSDTDYSQEFKGKATLTVDKSSSTAHMQLSSLTSEDSAVYYCAIWSYYYGTRLDYWGQGTTLTVSS

SEQ ID NO: 148 - protein, 119 aa, Mus musculus
QVQLQQPGSELVRPGTSVKLSCKASGYTFTSYWMHWVKQRPGQGLEWIGVIDPSDNNTHYNQKFTGKATLTVDTSSSTAYMHLSSLTSEDSAVFYCARNDYDGGYFDFWGQGTTLTVSS

SEQ ID NO: 149 - protein, 117 aa, Mus musculus
QVQLQQPGTELVKPGASVKVSCKASGYTFISYCMHWVKQRPGQGLDWIGNIDPNNGGPNYNEKFKTKATLTVDKSSSTAYMQLSSLTSEDSAVYYCARWGWDGFAYWGQGTLVTVSA

SEQ ID NO: 150 - protein, 117 aa, Mus musculus
QVQLQQPGTELVKPGASVKVSCKASGYTFISYWMHWVKQRPGQGLDWIGNIDPNNGGTNYNEKFKSKATLTVDKSSSTAYMQLSSLTSEDSAVYYCARWGWDGFAYWGQGTLVTVSA

SEQ ID NO: 151 - protein, 117 aa, Mus musculus
QVQLQQPGTELVKPGASVKVSCKASGYTFISYWMHWVKQRPGQGLEWIGNIDPNSGGTKYNEKFKSKAILTVDKSSSTAYMQLSSLTSEDSAVYYCTRWGWDGFAYWGQGTLVTVSA

SEQ ID NO: 152 - protein, 117 aa, Mus musculus
QVQLQQPGTELVKPGASVKVSCKASGYTFISYWMHWVKQRPGQGLEWIGNIDPNSGGTNYNEKFKSKAILTVDKSSSTAYMQLSSLTSEDSAVYFCARWGWDGFAYWGQGTLVTVSA

SEQ ID NO: 153 - protein, 117 aa, Mus musculus
QVQLQQPGTELVKPGASVKVSCKASGYTFISYWMHWVKQRPGQGLEWIGNIDPNSGGTNYNEKFKSKAILTVDKSSSTAYMQLSSLTSEDSAVYYCTRWGWDGFAYWGQGTLVTVSA

SEQ ID NO: 154 - protein, 117 aa, Mus musculus
QVQLQQPGTELVKPGASVKVSCKASGYTFISYWMHWVKQRPGQGLEWIGNIDPNSGITNYNEKFKSKAILTVDKSSSTAYMQLSSLTSEDSAVYYCARWGWDGFAYWGQGTLVTVSA

SEQ ID NO: 155 - protein, 117 aa, Mus musculus
QVQLQQPGTELVKPGASVKVSCKASGYTFISYWMHWVKQRPGQGLEWIGNIDPNSGTTNYNEKFKTKATLTVDKSSSTAYMQLSSLTSEDSAVYYCARWGWDGFAYWGQGTLVTVSA

SEQ ID NO: 156 - protein, 117 aa, Mus musculus
QVQLQQPGTELVKPGASVKVSCKASGYTFISYWMHWVKQRPGQGLEWIGNIDPNYGGPNYNEKFKTKATLTVDKSSSTAYMQLSSLTSEDSAVYYCARWGWDGFAYWGQGTLVTVSA

SEQ ID NO: 157 - protein, 117 aa, Mus musculus
QVQLQQPGTELVKPGASVKVSCKASGYTFISYWMHWVKQRPGQGLEWIGNIDPNYGGSNYNEKFKSKATLTVDKSSSTAYMQLSSLTSEDSAVYYCARWGWDGFAYWGQGTLVTVSA

SEQ ID NO: 158 - protein, 117 aa, Mus musculus
QVQLQQPGTELVKPGASVKVSCKASGYTFISYWMHWVKQRPGQGLEWIGNINPNNGGTNYNEKFKSKATLTVDKSSSTAYMQLSSLTSEDSAVYYCARWGWDGFAYWGQGTLVTVSA

SEQ ID NO: 159 - protein, 117 aa, Mus musculus
QVQLQQPGTELVKPGASVKVSCKASGYTFVSYWIHWVKQRPGQGLEWIGNIDPNNGGSNYNERFRTKATLTVDKSSSTAYMQLSSLASEDSAVYYCARWGWDGFAYWGQGTLVTVSA

SEQ ID NO: 160 - protein, 117 aa, Mus musculus
QVQLQQPGTELVKPGASVKVSCKASGYTFVSYWMHWVKQRPGQGLEWIGNIDPNNGGSNYNERFRTKATLTVDKSSSTAYMQLSSLASEDSAVYYCARWGWDGFAYWGQGTLVTVSA

SEQ ID NO: 161 - protein, 117 aa, Mus musculus
QVQLQQPGTELVKPGASVKVSCKTSGYTFISYWMHWVKQRPGQGLEWIGNIDPNSGTTNYNEKFKTKATLTVDKSSSTAYMQLSSLTPEDSAVYYCARWGWDGFAYWGQGTLVTVSA

SEQ ID NO: 162 - protein, 117 aa, Mus musculus
QVQLQQPGTELVKPGASVKVSCKTSGYTFISYWMHWVKQRPGQGLEWIGNIDPNSGTTNYNEKFKTKATLTVDKSSSTAYMQLSSLTSEDSAVYYCARWGWDGFAYWGQGTLVTVSA

SEQ ID NO: 163 - protein, 121 aa, Mus musculus
QVQLQQPGTELVMPGASVNLSCKASGYTFTSYWMHWVKQRPGQGLEWIGEIDPSDSYANYNQNFKGKATLTVDKSSITAYMQLSSLTSEDSAVYYCARNYGSNYVGYFDYWGQGTTLTVSS

SEQ ID NO: 164 - protein, 120 aa, Mus musculus
QVQLQQSGAELARPGASVKLSCKASGYTFRSYGLSWVKQRTGQGLEWIGEIYPRSGNTYYNEKFKDRVTLTADKSSSAAYMELRSLTSEDSAVYFCARSTAVVAGGFDYWGQGTALTVSS

SEQ ID NO: 165 - protein, 120 aa, Mus musculus
QVQLQQSGAELARPGASVKLSCKASGYTFTNYGISWVKQRTGQGLEWIGEIYPRSGNSYYNEKFKGKATLTADKSSSTAYMELRSLTSEDSAVYFCARSTAIVAGGFDYWGQGTTLTVSS

SEQ ID NO: 166 - protein, 121 aa, Mus musculus
QVQLQQSGAELARPGASVKLSCKASGYTFTSYGISWVKQRTGQGLEWIGEIYPRSGNTYYNEKFKGKATLTADKSSSTAYMELRSLTSEDSAVYFCARSTYGSSSYAMDYWGQGTSVTVSS

SEQ ID NO: 167 - protein, 112 aa, Mus musculus
QVQLQQSGAELMRPGASVTLSCKASGYTFPDYEMYWVKQTPVHGLEWIGVIDPETGGTAYNQKFEVKAILTADKSSNTAYMELRSLTSEDSAVYYCTRWAYWGQGTLVTVSA

SEQ ID NO: 168 - protein, 119 aa, Mus musculus
QVQLQQSGAELVKPGASVKLSCKASGYSITGYYIHWVKQRPGQGLEWIGWIYPGSGSIKYNEKFKGKATLTADTSSSTADMQLSSLTSEDSAVYYCAREAWGNWYFDVWGTGTTVTVSS

SEQ ID NO: 169 - protein, 115 aa, Mus musculus
QVQLQQSGAELVRPGASVTLSCKASGYTFSDYEMHWVKQTPVHGLEWIGVIDPETGDTAYNQKFKDKAVLTADKSSNTAYMELRSLTSEDSAVFFCTSNGYPEHWGQGTTLTVSS

SEQ ID NO: 170 - protein, 122 aa, Mus musculus
QVQLQQSGAELVRPGASVTLSCKASGYTFTDFEMHWVKQTPVHGLEWIGAIDPETGGTAYNQKFKGKAILTADRSSSTAYMELRSLTSEDSAVYYCTRGIYYGNYGSAMDYWGQGTSVTVSS

SEQ ID NO: 171 - protein, 112 aa, Mus musculus
QVQLQQSGAELVRPGASVTLSCKASGYTFTDYEMYWVKQTPVHGLEWIGVIDPETGGTAHDQKFKGKAILTADKSSSTAYMELRSLTSEDSAVYYCTRWAFWGQGTLVTVSA

SEQ ID NO: 172 - protein, 118 aa, Mus musculus
QVQLQQSGAELVRPGTSVKVSCKASGYAFSNYLIEWVKQRPGQGLEWIGVINPGNGGTTYNEKFKDKATLTADRSSTTAYMQLSSLTSEDSAVFFCARTAYGSPLDYWGQGTTLTVSS

SEQ ID NO: 173 - protein, 118 aa, Mus musculus
QVQLQQSGAELVRPGTSVKVSCKASGYAFTNYLIEWAKQRPGQGLEWIGVINPGSGATTYNEKFKGRATLTADMSSTTAYMQLSSLTSEDSAVFFCARTAYGSPLDCWGQGTTLTVSS

SEQ ID NO: 174 - protein, 121 aa, Mus musculus
QVQLQQSGAELVRPGTSVKVSCKASGYAFTNYLIEWVKQRPGQGLEWIGVINPGSGATNYNEKFKGKATLTADKSSSTAYMQLRSLASEDSAVYFCARRTTVVAPYAMDYWGQGTSVTVSS

SEQ ID NO: 175 - protein, 118 aa, Mus musculus
QVQLQQSGAELVRPGTSVKVSCKASGYAFTNYLIEWVKQRPGQGLEWIGVISPGSGATTYNEKFKGKATLTADRSSTTAYMQLSSLTSEDSAVYFCARSAYGSPLDFWGQGTTLTVSS

SEQ ID NO: 176 - protein, 118 aa, Mus musculus
QVQLQQSGAELVRPGTSVKVSCKASGYAFTNYLIEWVKQRPGQGLEWIGVISPGSGGTTYNEKFKGKATLTADKSSSTAYMQFSSLTSEDSAVYFCARSAYGHVFDYWGQGTTLTVSS

SEQ ID NO: 177 - protein, 118 aa, Mus musculus
QVQLQQSGAELVRPGTSVRVSCTASGYAFTNYLIEWVKQRPGQGLEWIGVISPGSGGTTYNEKFKGKATLTADKSSSTAYMQFSSLTSEDSAVYFCARSAYGHVFDYWGQGTTLTVSS

SEQ ID NO: 178 - protein, 120 aa, Mus musculus
QVQLQQSGPELARPGASVKLSCKASGYTFRSNGISWVKQRTGQGLEWIGEIYPRSGNIYYNEKVKDKATLTADKSSNTAYMAVRSLTSEDSAVYFCARSTTVVAGGFDFWGQGTVLTVSS

SEQ ID NO: 179 - protein, 119 aa, Mus musculus
QVQLQQSGPELVKPGASVKISCKASGYSFTGYYIHWMKQRPGKGLEWIGWIYPGSGNTKYNEKFKGKATLTADTSSSTADMQLSSLTSEDSAVYYCAREAWGNWYFDVWGTGTTVTVSS

SEQ ID NO: 180 - protein, 119 aa, Mus musculus
QVQLQQSGPELVKPGASVKISCKASGYSFTGYYIHWVKQRPGQGLEWIGWIYPGSGNTKYNEKFKGKATLTADTSSSTADMQLSSLTSEDSAVYYCAREAWGNWYFDVWGTGTTVTVSS

SEQ ID NO: 181 - protein, 122 aa, Mus musculus
QVQLQQSGPELVKPGASVKISCKASGYSFTGYYIHYIHWVKQRPGQGLEWIGWIYPGSGNTKYNEKFKVKATLTADTSSSTADMQLSSLTSEDSAVYYCAREAWGNWYFDVWGTGTTVTVSS

SEQ ID NO: 182 - protein, 121 aa, Mus musculus
QVQLRQPGAELVMPGASVKLSCKASGYTFNTYWMHWVKQRPGQGLEWIGEIDPSDSYTNYNQNFKGKATLTVDKSSSTAYLQLSSLTSEDSAVYYCARNYGSNYVGYFDYWGQGTTLTVSS

SEQ ID NO: 183 - protein, 120 aa, Mus musculus
QVQLRQSGAELARPGASVKLSCEASGYTFTSFGITWVKQRPGQGLEWIGEIYPRSGNIYHNEKLKGKVTLTADKSSSTVYMELRSLTSEDSAVYFCARSTAVVAGGFDYWGQGTTLTVSS

SEQ ID NO: 184 - protein, 120 aa, Mus musculus
QVQLRQSGAELARPGASVKLSCKASGYTFTNYGITWVKQRTGQGLEWIGEIYPRSGNIYYNEKFKGKVTLTADKSSSTAYMELRSLTSEDSAVYFCARSTAVVAGGFDYWGQGTTLTVSS

SEQ ID NO: 185 - protein, 120 aa, Mus musculus
QVQLRQSGAELARPGASVKLSCKASGYTFTSYGITWVKQRTGQGLEWIGEIYPRSGNIYYNEKFKGKVTLTADKSSSTAYMELRSLTSEDSAVYFCARSTAVVAGGFDYWGQGTTLTVSS

SEQ ID NO: 186 - protein, 123 aa, Mus musculus
QVQLRQSGAELVRPGTSVKLSCKASGYTLTDYYINWVKQRPGQGLEWIARIYPGNGNTYYNEKFKGKATLTAEKSSSTVNMQVSSLTSEDSAVYFCARGGSSDRIYYYAMDYWGQGTSVTVSS

SEQ ID NO: 187 - protein, 112 aa, Mus musculus
DAVMTQTPLSLPVSLGDQASISCRSSQSLENSDGNTYLNWYFQKTGQSPQLLIYRVSNRFSGVLDRFSGSGSGTDFTLKISRVEAEDLGVYFCLQVTHVPPTFGAGTKLELK

SEQ ID NO: 188 - protein, 112 aa, Mus musculus
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQITHVPYTFGGGTKLEIK

SEQ ID NO: 189 - protein, 112 aa, Mus musculus
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQLTHVPYTFGGGTKLEIK

SEQ ID NO: 190 - protein, 112 aa, Mus musculus
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPYTFGGGTKLEIK

SEQ ID NO: 191 - protein, 112 aa, Mus musculus
DAVMTQTPLSLPVSLGDQASISCRSSQSLENSNGNTYLNWYLQKPGQSPQLLIYRVSTRFSGVLDRFSGSGSGTDFTLKISRVEAEDLGVYFCLQLTHVPYTFGGGTKLEIK

SEQ ID NO: 192 - protein, 112 aa, Mus musculus
DAVMTQTPLSLPVSLGDQASISCRSSQSLENSNGNTYLNWYLQKPGQSPQLLIYRVSTRFSGVLDRFSGSGSGTDFTLKISRVEAEDLGVYFCLQVTHVPYTFGGGTKLEIK

SEQ ID NO: 193 - protein, 112 aa, Mus musculus
DAVMTQTPLSLPVSLGDQASISCRSSQSLENSYGNTYLNWYLQKPGQSPQLLIYRVSTRFSGVLDRFSGSGSGTDFTLKISRVEAEDLGVYFCLQVTHVPYTFGGGTKLEIK

SEQ ID NO: 194 - protein, 112 aa, Mus musculus
DAVMTQTPLSLPVSLGDQVSISCRSSQSLENSNGNTYLNWYLQKPGQSPQLLIYRISTRFSGVLDRFSGSGSGTDFTLKISRVEAEDLGVYFCLQVTHVPYTFGGGTKLEIK

SEQ ID NO: 195 - protein, 112 aa, Mus musculus
DFVMTQTPLSLPVSLGDQASISCRSSQSLLHSNGNTYLHWYLQKPGQSPKLLIYKVSNRFSGVPDRFSGGGSGTDFTLKISRVEAEDLGVYFCSQTTHVPWTFGGGTKLEIK

SEQ ID NO: 196 - protein, 107 aa, Mus musculus
DIKMTQSPSSIYASLGERVTITCKASQDINSYLKWFQQKPGKSPKTLIYRANRLVDGVPSRFSGSGSGQDYSLTISSLEYEDMGIYYCLQYDEFPYTFGGGTKLEIK

SEQ ID NO: 197 - protein, 107 aa, Mus musculus
DIKMTQSPSSMYASLGERVTITCKASQDIDRYLNWFQQKPGKSPKTLIYRATRLVDGVPSRFSGSGSGQDYSLTISSLEYEDMGIYYCLQYDEFPYTFGGGTKLEIK

SEQ ID NO: 198 - protein, 107 aa, Mus musculus
DIKMTQSPSSMYASLGERVTITCKASQDIDSYLNWFQQKPGKSPKTLIYRATRLVDGVPSRFSGSGSGQDYSLTISSLEYEDMGIYYCLQYDEFPYTFGGGTKLEIK

SEQ ID NO: 199 - protein, 107 aa, Mus musculus
DIKMTQSPSSMYASLGERVTITCKASQDINRYLSWFQQKPGKSPKTLIYRVNRLVDGVPLRFSGSGSGQDYSLTISSLDYEDMGIYYCQQYDEFPYTFGGGTKLEIK

SEQ ID NO: 200 - protein, 107 aa, Mus musculus
DIKMTQSPSSMYASLGERVTITCKASQDINRYLSWLQQKAGKSPKTLIYRENRLVEGVPSRFSGSGSGQDYSLTISSLEYEDMGIYYCLQYDEFPYTFGGGTKLEIK

SEQ ID NO: 201 - protein, 107 aa, Mus musculus
DIKMTQSPSSMYASLGERVTITCKASQDINSYLKWFQQKPGKSPKTLIYRANRLVDGVPSRFSGSGSGQDYSLTISSLEYEDMGIYYCLQYDEFPYTFGGGTKLEIK

SEQ ID NO: 202 - protein, 107 aa, Mus musculus
DIKMTQSPSSMYASLGERVTITCKASQDINSYLRWFQQKPGKSPKTLIYRANRLVDGVPSRFSGSGSGQDYSLNISSLEYEDMGIYYCLQYDEFPYTFGGGTKLEIK

SEQ ID NO: 203 - protein, 107 aa, Mus musculus
DIKMTQSPSSMYASLGERVTITCKASQDINSYLSWFQLKPGKSPKTLIYRANRLVDGVPSGFSGSGSGQDYSLTISSLEYEDMGIYYCLQYDEYPYTFGGGTKLEIK

SEQ ID NO: 204 - protein, 107 aa, Mus musculus
DIKMTQSPSSMYASLGERVTITCKASQDINSYLSWFQQKPGKSPKTLIYRANRLLDGVPSRFSGSGSGQDYSLTISSLEYEDMGIYYCLQYDEYPYTFGGGTKLEIK

SEQ ID NO: 205 - protein, 107 aa, Mus musculus
DIKMTQSPSSMYASLGERVTITCKASQDINSYLSWFQQKPGKSPKTLIYRANRLVDGVPSRFSGSGSGQDYSLTISSLEYEDMGIYYCLQYDEYPYTFGGGTKLEIK

SEQ ID NO: 206 - protein, 107 aa, Mus musculus
DIKMTQSPSSMYASLGERVTITCKASQDINSYLSWFQQKPGKSPKTLIYRTNRLVDGVPSRFSGSGSGQDYSLTISSLEYEDMGIYYCLQYDEYPYTFGGGTKLEIK

SEQ ID NO: 207 - protein, 107 aa, Mus musculus
DIKMTQSPSSMYASRGERVTITCKASQDINRYLSWLQQKPGKSPKTLIYRANRLVDGVPSRFSGSGSGQDYSLTISSLEYEDMGIYYCLQYDEFPYTFGGGTKLEIK

SEQ ID NO: 208 - protein, 107 aa, Mus musculus
DILLTQSPAILSVSPGERVSFSCRASQSIGTSIHWFQQRTNGSPRLLIKYASESISGIPSRFSGSGSGTDFTLSINSVESEDIADYYCQQSNSWPYTFGGGTKLEIK

SEQ ID NO: 209 - protein, 107 aa, Mus musculus
DILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 210 - protein, 107 aa, Mus musculus
DILLTQSPDILSVSPGERVSFSCRASQNIGTSIHWYQQRTNGSPRLLIKYASESISGISSRFSGSGSGTDFTLSINSVESEDIADYYCQQSNNWPFTFGSGTKLEIK

SEQ ID NO: 211 - protein, 107 aa, Mus musculus
DILMTQSPSSMSVSLGDRVSIICHASQDIRSNIGWLQQKPGKSLKGLIYHGTNLEDGVPSRFSGSGSGADYSLTISSLESEDFADYYCVQYVQFPRTFGGGTKLEIK

SEQ ID NO: 212 - protein, 107 aa, Mus musculus
DILMTQSPSSMSVSLGDTISIICHASQDIRSNIGWLQQKPGKSFKGLIYHGSNLEDGVPSRFSGSGSGADYSLTISSLESEDFADYYCVQYIQFPRTFGGGTKVEIK

SEQ ID NO: 213 - protein, 107 aa, Mus musculus
DILMTQSPSSMSVSLGDTVSIICHASQDIRSNIGWLQQKPGKSFKGLIYHGTNLEDGVPSRFSGSGSGADYSLTISSLESEDFADYYCVQYIQFPRTFGGGTKLEIK

SEQ ID NO: 214 - protein, 107 aa, Mus musculus
DILMTQSPSSMSVSLGDTVSIICHASQDIRSNIGWLQQKPGKSFKGLIYHGTNLEDGVPSRFSGSGSGADYSLTISSLESEDFADYYCVQYVQFPRTFGGGTKLEIK

SEQ ID NO: 215 - protein, 107 aa, Mus musculus
DILMTQSPSSMSVSLGDTVSIICHASQDIRSNIGWLQQKPGKSFKGLIYHGTNLEDGVPSRFSGSGSGADYSLTISSLESEDFADYYCVQYVQFPRTFGGGTKLEIN

SEQ ID NO: 216 - protein, 107 aa, Mus musculus
DILMTQSPSSMSVSLGDTVSIICHASQDIRSNIGWLQQKPGKSFKGLIYHGTNLEDGVPSRFSGSGSGTDYSLTISSLESEDFAEYYCVQYVQFPRTFGGGTKLEIK

SEQ ID NO: 217 - protein, 107 aa, Mus musculus
DILMTQSPSSMSVSLGDTVSITCHASQGINNNVGWLQQKPGKSFKGLIYHGTNLEDGVPSRFSGSGSGADYSLTIYSLESEDFADYYCVQYDQFPPTFGGGTKLEIK

SEQ ID NO: 218 - protein, 107 aa, Mus musculus
DILMTQSPSSMSVSLGDTVTIICHASQDIRSNIGWLRQKPGTSFKGLIYHGTNLEDGVPSRFSGSGSGADYSLTISSLESEDFADYYCVQYVQFPRTFGGGTKLEIK

SEQ ID NO: 219 - protein, 107 aa, Mus musculus
DIQLTQTTSSLSASLGDRVTINCRASQDISNYLNWYQQKPDGTVKLLIYYTSRLLSGVPSSFSGSGSGTDYSLTISSLEQEDIATYFCQQGKALPWTFGGGTKLEIK

SEQ ID NO: 220 - protein, 107 aa, Mus musculus
DIQMNQSPSSLSASLGDRITINCHASQNINVWLSWYQQKPGNIPKLLIYKASNLHTGVPLRFSGSGSVTVFTLTISSLQPEDIATYYCQQGQNYPYTFGGGTKLEIK

SEQ ID NO: 221 - protein, 107 aa, Mus musculus
DIQMNQSPSSLSASLGDRITINCHASQNINVWLSWYQQKPGNIPKLLIYKASNLHTGVPSRFSGSGSVTVFTLTISSLQPEDIATYYCQQGQNYPYTFGGGTKLEIK

SEQ ID NO: 222 - protein, 107 aa, Mus musculus
DIQMNQSPSSLSASLGDTINITCHASQNINVWLSWYQQKPGNIPKLLIYKASNLHTGVPLRFSGSGSGTGFTLTISSLQPEDIATYYCQQGQSYPLTFGSGTKLEIK

SEQ ID NO: 223 - protein, 107 aa, Mus musculus
DIQMNQSPSSLSASLGDTISITCHASQNINVWLSWYQQKPGNIPKLLIYKASILHTGVPSRFSGSGSVTGFTLTISSLQPEDIASYYCQQGQNYPYTFGGGTKLEIK

SEQ ID NO: 224 - protein, 107 aa, Mus musculus
DIQMNQSPSSLSASLGDTITITCHASQNINVWLGWYQQKPGNIPKLLIYKASNLHTGVPSRFSGSGSVTSFTLTISSLQPEDIATYYCQQGQSYPYTFGGGTKLEIK

SEQ ID NO: 225 - protein, 107 aa, Mus musculus
DIQMNQSPSSLSASLGDTITITCHASQNINVWLSWYQQKPGDIPKLLIYKASNLHTGVPSRFSGSGSGTGFTLTISSLQPEDIATYYCQQGQSYPLTFGSGTKLEIK

SEQ ID NO: 226 - protein, 107 aa, Mus musculus
DIQMNQSPSSLSASLGDTITITCHASQNINVWLSWYQQKPGNILKLLIYKASNLHTGVPSRFSGSGSGTGFTLTISSLQPEDIATYYCQQGQSYPLTFGSGTKLEIK

SEQ ID NO: 227 - protein, 107 aa, Mus musculus
DIQMNQSPSSLSASLGDTITITCHASQNINVWLSWYQQKPGNIPKLLIYKASNLHTGVPSRFSGSGSGTGFTLAISSLQPEDIATYYCQQGQSYPLTFGSGTKLALK

SEQ ID NO: 228 - protein, 107 aa, Mus musculus
DIQMNQSPSSLSASLGDTITITCHASQNINVWLSWYQQKPGNIPKLLIYKASNLHTGVPSRFSGSGSGTGFTLTISSLQPEDIATYYCQQGQNFPYTFGGGAKLEIK

SEQ ID NO: 229 - protein, 107 aa, Mus musculus
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGSGTKLEIK

SEQ ID NO: 230 - protein, 107 aa, Mus musculus
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGSGTKLEVK

SEQ ID NO: 231 - protein, 107 aa, Mus musculus
DIQMNQSPSSLSASLGDTITITCHASQNINVWLSWYQQKPGNIPKLLIYKASNLHTGVPSRFSGSGSVTGFTLTISSLQPEDIATYYCQQGQNFPYTFGGGTKLEII

SEQ ID NO: 232 - protein, 107 aa, Mus musculus
DIQMNQSPSSLSASLGDTITITCHASQNINVWLSWYQQKPGNIPKLLIYKASNLHTGVPSRFSGSGSVTGFTLTISSLQPEDIATYYCQQGQNFPYTFGGGTKLEIK

SEQ ID NO: 233 - protein, 107 aa, Mus musculus
DIQMNQSPSSLSASLGDTITITCHASQNINVWLSWYQQKPGNIPKLLIYKASNLHTGVPSRFSGSGSVTGFTLTISSLQPEDIATYYCQQGQNIPYTFGGGTQLEIK

SEQ ID NO: 234 - protein, 107 aa, Mus musculus
DIQMNQSPSSLSASLGDTITITCHASQNINVWLSWYQQKPGNIPKLLIYKASNLHTGVTSRFSGSGSGTGFTLTISSLQPEDIATYYCQQGQSYPLTFGSGTKLEIK

SEQ ID NO: 235 - protein, 107 aa, Mus musculus
DIQMNQSPSSLSASLGDTITITCHASQTINVWLSWYQQKPGNIPKLLIYKASKMHTGVPSRFSGSGSGTGFTLNISSLQPEDIATYYCHQGQSYPYTFGGGTKLEIK

SEQ ID NO: 236 - protein, 107 aa, Mus musculus
DIQMNQSPSSLSASLGDTITITCHASQTINVWLSWYQQKPGNIPKLLIYKASNLHTGVPSRFSGSGSGTGFTLNISSLQPEDIATYYCHQGQSYPYTFGGGTKLEIK

SEQ ID NO: 237 - protein, 107 aa, Mus musculus
DIQMNQSPSSLSASLGDTITITCHVSQNINVWLSWYQQKPGNIPKLLIYKASNLHTGVTSRFSGSGAGTGFTLTISSLQPEDIATYYCQQGQNYPYTFGGGTKLEIK

SEQ ID NO: 238 - protein, 107 aa, Mus musculus
DIQMNQSPSSLSASLGDTITITCHVSQNINVWLSWYQQKPGNIPKLLIYKASNLHTGVTSRFSGSGAGTGFTLTISSLQPEDLATYYCQQGQNYPYTFGGGTKLEIK

SEQ ID NO: 239 - protein, 107 aa, Mus musculus
DIQMNQSPSSLSASLGDTITITCRASQTINIWLSWYQQKPGNIPKLLIYKASTLHTGVPSRFSGSGSGTGFTLNISSLQPEDIATYYCHQGQSYPYTFGGGTKLEIK

SEQ ID NO: 240 - protein, 107 aa, Mus musculus
DIQMNQSPSSLSASLGDTVTITCHASQTINVWLSWYQQKPGNIPKLLIYKASNLHTGVPSRFSGSGSGTGFTLTISSLQPEDIATYYCQQGQNYPYTFGGGTKLEIK

SEQ ID NO: 241 - protein, 107 aa, Mus musculus
DIQMTQSPASLSASVGETVTITCRASGNIHNYLAWYQQKQGKSPQLLVYNAETLADGVPSRFSGSGSGTQYSLKINSLQPEDFGTYYCQHFWSTPLTFGAGTKLELK

SEQ ID NO: 242 - protein, 107 aa, Mus musculus
DIQMTQSPSSLSASLGERVSLTCRASQDIGSSLNWLQQEPDGTIKRLIYATSSLDSGVPKRFSGSRSGSDYSLTISSLESEDFVDYYCLQYASSPFTFGSGTKLEIK

SEQ ID NO: 243 - protein, 107 aa, Mus musculus
DIQMTQSPSSLSASLGERVSLTCRASQEISGYLSWLQQKPDGTIKRLISRASSLDFGVPKRFRGSRSGSDYSLTISSLESEDFADYYCLQYANYPLTFGAGTKLELK

SEQ ID NO: 244 - protein, 107 aa, Mus musculus
DIQMTQSTSSLSASLGDRVTISCRASQDITNYLNWYQQKPDGTVKLLIYHTSRLHAGVPSRFSGSGSGTDYSLTISNLDQEDIATYFCQQGNALPWTFGGGTKLEIK

SEQ ID NO: 245 - protein, 107 aa, Mus musculus
DIQMTQTASSLSASLGDRVTISCRASQDINNYLNWYQQKPDGTVKLLIYYTSRLHSGVPSSFSGSGSGTDYSLTISNLEQEDIATYFCQQGNALPWTFSGGTKLEIK

SEQ ID NO: 246 - protein, 107 aa, Mus musculus
DIQMTQTTSSLSASLGDRVTISCRASQDINNYLNWYQQKPDETVKLLIYYTSRLHSGVPSRFSGSGSGTDYSLTISNLDQEDFATYFCQQGYMLPLTFDAGTKLELK

SEQ ID NO: 247 - protein, 107 aa, Mus musculus
DIQMTQTTSSLSASLGDRVTISCRASQDISNYLNWYQQKPDGTVELLIYYTSRLHSGVPSRFSGSGSGTDYSLTISNLEQEDIATYFCQQGNTLPRTFGGGTKLEIK

SEQ ID NO: 248 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLDQEDIATYFCQQEKTLPWTFGGGTKLEIK

SEQ ID NO: 249 - protein, 107 aa, Mus musculus
DIQMTQTTSSLSASLGDRVTISCRASQDISNYLNWYQQKPDGTVKLLIYYTSRLHSGVPSSFSGSGSGTDYSLTISNLAQEDIATYFCQQGNALPWTFGGGTKLEIK

SEQ ID NO: 250 - protein, 107 aa, Mus musculus
DIQMTQTTSSLSASLGDRVTISCRASQDISNYLNWYQQKPDGTVKLLIYYTSRLHSGVPSSFSGSGSGTDYSLTISNLEQEDIAIYFCQQANALPWTFGGGTKLEIK

SEQ ID NO: 251 - protein, 107 aa, Mus musculus
DIQMTQTTSSLSASLGDRVTISCRASQDISNYLNWYQQKPDGTVKLLIYYTSRLHSGVPSSFSGSGSGTDYSLTISNLEQEDIATYFCQQANALPWTFGGGTKLEIK

SEQ ID NO: 252 - protein, 107 aa, Mus musculus
DIQMTQTTSSLSASLGDRVTISCRASQDISNYLNWYQQKPDGTVKLLIYYTSRLLSGVPSRFSGSGSGTDYSLTISNLEQEDIATYFCQQGNTLPWTFGGGTNLEIK

SEQ ID NO: 253 - protein, 107 aa, Mus musculus
DIQMTQTTSSLSASLGDRVTISCRASQDISNYLNWYQQKPDGTVKLLIYYTSRLPSGVPSSFSGSGSGTDYSLTISNLEQEDIATYFCQQAKALPWTFGGGTKLEIK

SEQ ID NO: 254 - protein, 107 aa, Mus musculus
DIQMTQTTSSLSASLGDRVTISCRASQDISNYLNWYQQKRDGTVKLLIYYTSRFHSGVPSRFSGSGSGTDYSLTISNLEQEDIATYFCQQGNTLPWTFGGGTKLEIK

SEQ ID NO: 255 - protein, 107 aa, Mus musculus
DIQMTQTTSSLSASLGDRVTISCRASQDITNYLNWYQQRPDGTVKLLIYYTSRLHSGVPSRFSGSGSGTDYSLTISNLAQGDIATYFCQQASTLPWTFGGGTKLDIK

SEQ ID NO: 256 - protein, 107 aa, Mus musculus
DIQMTQTTSSLSASLGDRVTISCRASQGISNYLNWYQQKPDGTVKLLIYYTSRFHSGVPSRFSGSGSGTDYSLTISNLEQEDIATYFCQQGNSLPWTFGGGTKLEIK

SEQ ID NO: 257 - protein, 107 aa, Mus musculus
DIQMTQTTSSLSASLGDRVTISCRSSQDISNYLNWYQQKPDGTVKLLIYYTSRLHSGVPSSFSGSGSGTDYSLTISNLEQEDIATYFCQQANALPWTFGGGTKLEIK

SEQ ID NO: 258 - protein, 107 aa, Mus musculus
DIQVTQSSPFLSVSLGGRVIISCKASDHINNWLAWYQQKPGNAPRLLISGAINLETGVPSRFSGSGSGKDYTLSISSLQTEDVATYYCQQYWNTPYTFGGGTKLEIK

SEQ ID NO: 259 - protein, 107 aa, Mus musculus
DIRMTQSPSSIYASLGERVTITCKASQDINSYLSWFQQKPGKSPETLIYRTNRLVDGVPSRFSGSGSGQDYSLTISSLEYEDMGIYYCLQYDEYPYTFGGGTKLEIK

SEQ ID NO: 260 - protein, 111 aa, Mus musculus
DIVLTQSPASLAVSLGQRATISCRASESVDSYGNSFMHWYQQKPGQPPKLLIYRASNLESGIPARFSGSGSRTDFTLTIYPVEADDVATYYCQQSNEDPYTFGGGTKLEIK

SEQ ID NO: 261 - protein, 108 aa, Mus musculus
DIVLTQSPATLSVTPGDSVSLSCRASQSINTDLHWYQQKSHESPRLLIKYASQSISGIPSRFSGSGSGTDFSLSINSVETEDFGIYFCQQSHTWPQYTFGGGTKLEIK

SEQ ID NO: 262 - protein, 108 aa, Mus musculus
DIVLTQSPATLSVTPGDSVSLSCRASQSISNNLHWYQQKSHESPRLLIKYASQSISGIPSRFSGSGSGTDFTLSINSVETEDFGIYFCQQSNSWPQYTFGGGTKLEIK

SEQ ID NO: 263 - protein, 108 aa, Mus musculus
DIVLTQSPATLSVTPGDSVSLSCRASQTISNNLHWYQQKSHESPRLLIKYASQSISGIPSRFSGSGSGTDFTLRINSVETEDFGMYFCQQSNSWPQYTFGGGTKLEIK

SEQ ID NO: 264 - protein, 107 aa, Mus musculus
DIVLTQSQKFMSASVGDRVSVTCKASQNVGTNVAWYQQRPRQSPKALIYSASYRYSGVPDRFTGSGSGTDFTLAISNVQSEDLAEYFCQQYNSYPYTFGGGTKLEII

SEQ ID NO: 265 - protein, 113 aa, Mus musculus
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPRTFGGGTKLEIK

SEQ ID NO: 266 - protein, 107 aa, Mus musculus
DIVMTQSHKFMSTSVGDRVSITCKASQDVGAAVAWYQQKPGQSPKLLIYSASYRYTGVPDRFAGSGSGTDFTFTISSVQAEDLAVYYCQQHFNTPFTFGSGTNLEIK

SEQ ID NO: 267 - protein, 107 aa, Mus musculus
DIVMTQSHKFMSTSVGDRVSITCKASQGVSTAVAWYQQEPGQSPKLLIYSASYRYTGVPDRFTGSGSGTDFTFTISSVQAEDLAVYYCQQYYNTPYTFGGGTKLEIK

SEQ ID NO: 268 - protein, 107 aa, Mus musculus
DIVMTQSHKFMSTSVGNRVSITCKVSQDVSTAVAWYQQKPGQSPKLLIYSASYRYTGVPDRFAGSGSGTDFTFTISSVQAEDLAVYYCQQHFNTPFTFGSGTNLEIK

SEQ ID NO: 269 - protein, 106 aa, Mus musculus
DIVMTQSHKFMSTSVRDRVSITCKASQDVSTAVAWYQQKPGQSPKLLIYSASYRYTGVPDRFTGSGSGTDFTFTISSVQAEDLAVYYCQQHYSTPTFGGGTKLEIQ

SEQ ID NO: 270 - protein, 113 aa, Mus musculus
DIVMTQSPSSLTVTAGEKVTMSCKSSQSLFNSRTQKNSLTWYQQKPGQPPKLLIFWASTRESGVPDRFTGSASGTDFTLTLSGVQAEDLAIYYCQNDYTYPWTFGGGTKLEIK

SEQ ID NO: 271 - protein, 113 aa, Mus musculus
DIVMTQSPSSLTVTAGEKVTMSCKSSQSLLNSKNQKNFLTWYQQKPGQPPKLLIYWASTRESGVPDRFTGSASGTDFTLTISSVQAEDLAVYYCQNDYDYPYTFGGGTKLEIK

SEQ ID NO: 272 - protein, 113 aa, Mus musculus
DIVMTQSPSSLTVTAGEKVTMSCKSSQSLLNSKNQKNFLTWYQQKPGQPPKLLIYWASTRESGVPDRFTGSASGTDFTLTISSVQAEDLAVYYCQNDYSYPYTFGGGTKLEIK

SEQ ID NO: 273 - protein, 113 aa, Mus musculus
DIVMTQSPSSLTVTAGEKVTMSCKSSQSLLNSRIQKNSLTWYQQKPGQPPKLLIYWASTRESGVPDRFTASGSGTDFTLTISSVQAEDLAVYYCQNDYTYPWTFGGGTKLEIK

SEQ ID NO: 274 - protein, 113 aa, Mus musculus
DIVMTQSPSSLTVTAGEKVTMSCKSSQSLLNSRTQKNSLTWYQQKPGQPPKLLIYWASTRESGVPDRFTGSGSGTDFTLTISSVQAEDLAIYYCQNDYTYPWTFGGGTELEIK

SEQ ID NO: 275 - protein, 113 aa, Mus musculus
DIVMTQSPSSLTVTTGEKVTVNCKSSQSLLNSRIQKNSLTWYQQKPGQPPKLLIFWASTRESGVPDRFTGSESGTDFTLTISSVQAEDLAIYYCQNDYNYPWTFGGGTKLEIK

SEQ ID NO: 276 - protein, 113 aa, Mus musculus
DIVMTQSPSSLTVTTGEKVTVNCKSSQSLLNSRIQKNSLTWYQQKPGQPPKLLIYWSSTRESGVPDRFTGSGSGTDFTLTISSVQAEDLAIYYCQNDYNYPWTFGGGTKLEIK

SEQ ID NO: 277 - protein, 107 aa, Mus musculus
DIVMTQSQKFMSTSVGDRVSVTCKASQNVGTNVAWYQQKPGQSPKALIYSASYRYSGVPDRFTGSGSGTDFTLTISNVQSEDLAEYFCQQYNTYPLTFGAGTKLELK

SEQ ID NO: 278 - protein, 112 aa, Mus musculus
DVLMTQAPPSLPVSLGDQASISCRSSQNIVHSNGNTYLEWYLQRPGQSPKLLIYKVSNRFSGVPDRFSGSGSGTDFTLKISRVEAEDLGVYYCCQGSHVPYTFGGGTKLEIK

SEQ ID NO: 279 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHAPLTFGAGTKLELK

SEQ ID NO: 280 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 281 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 282 - protein, 112 aa, Mus musculus
DVLMTQTPLSLPVSLGDQASISCRSSQTIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 283 - protein, 112 aa, Mus musculus
DVLMTQTPLSLPVSLGDQASISCRSSQTIVHSNGNTYLEWYLQTPGQSPKLLIYKVSNRISGVPDRFSGSGSGTDFTLKISRVEAEDLGVYYCFQGSHVPFTFGSGTKLEIK

SEQ ID NO: 284 - protein, 112 aa, Mus musculus
DVLMTQTPLSLPVSLGDQVSISCRSSQSIVHSNGNTYLEWYLQKPGQSPNLLIYKVANRLSGVPDRFSGSGSGTDFTLKISRVEAEDLGVYYCFQGSHVPFTFGSGTKLEIK

SEQ ID NO: 285 - protein, 112 aa, Mus musculus
DVLMTQTPLSLTVSLGDQASISCRSSQSIVHSNGNTYLEWFLQKPGQSPKLLIYKVSNRFSGVPDRFSGSGSGTDFTLKISRVEAEDLGVYYCFQGSHVPWTFGGGTKLEIK

SEQ ID NO: 286 - protein, 112 aa, Mus musculus
DVLMTQTPPSLPVSLGDQASISCRSSQNIVHSNGNTYLEWYLQRPGQSPKLLIYKVSNRFSGVPDRFSGSGSGTDFTLKISRVEAEDLGVYYCCQGSHVPYTFGGGTKLEIK

SEQ ID NO: 287 - protein, 108 aa, Mus musculus
DVVLTQSPATLSVTPGDSVSLSCRASQSISNNLHWYQQKSHESPRLLIKYTSQSISGIPSRFSGSGSGTDFTLSINSVETEDFGMYFCQQSNSWPQYTFGGGTKLEIK

SEQ ID NO: 288 - protein, 112 aa, Mus musculus
DVVLTQTPLTLSVTIGQPASISCKSSQSLLYSNGKTYLNWLLQRPGQSPKRLIYLVSTLDSGVPDRFTGSGSGTDFTLKISRVEAEDLGVYYCVQGTHFPLTFGGGTKLEIK

SEQ ID NO: 289 - protein, 112 aa, Mus musculus
DVVMTQIPLSLPVSLGDQASISCRSSQSLVHSNGNTYLHWYLQKPGQSPKLLIYTVSNRFSGVPDRFSGSGSGTDFTLKISRLEAEDLGVYFCSQTTHVPWTFGGGTKLEIK

SEQ ID NO: 290 - protein, 113 aa, Mus musculus
DVVMTQIPLTLSVTIGQPASISCKSSQSLLDSDGKTYLNWLLQRPGQSPKRLIFLVSKLDSGVPDRFTGSGSGTDFTLKISRVEAEDLGVFYCWQGTHFPTWTFGGGTKLEIK

SEQ ID NO: 291 - protein, 112 aa, Mus musculus
DVVMTQSPLSLPVSLGDQASISCRSSQSLVHSNGITYLHWYLQKPGQSPKLLIYKVSNRFSGVPDRFSGSGSGTDFTLKISRVEAEDLGVYFCSQSTHVPWTFGGGTKLELK

SEQ ID NO: 292 - protein, 112 aa, Mus musculus
DVVMTQSPLSLPVSLGDQASISCRSSQSLVHSNGITYLHWYLQKPGQSPNLLIYKVSNRFSGVPDRFSGSGSGTDFTLKISRVEAEDLGVYFCSQTTHVPWTFGGGTKLEIK

SEQ ID NO: 293 - protein, 113 aa, Mus musculus
DVVMTQSPSSLTVTTGERVTVNCKSSQSLLNSRIQKNSLTWYQQKPGQPPKLLIYWASTRESGVPDRFTGSGSGTDFTLTISSVQAEDLAIYYCQNDYNYPWTFGGGTKLEIK

SEQ ID NO: 294 - protein, 112 aa, Mus musculus
DVVMTQTPLSLPGSLGDQASISCRSSQSLVHSNGNTYLHWYLQKSGQSPKLLIYKVSNRFSGVPDRFSGSGSGTDFTLKISRVEAEDLGLYFCSQTTHVPWTFGGGTKLEIK

SEQ ID NO: 295 - protein, 112 aa, Mus musculus
DVVMTQTPLSLPVSLGDHASISCRSSQSLVHSNGHTYLHWYLQKPGQSPKLLIDKVSNRFSGVPDRFSGSGSGTDFTLKISRVEAEDLGVYFCSQSTHVPWTFGGGTKLEIK

SEQ ID NO: 296 - protein, 112 aa, Mus musculus
DVVMTQTPLSLPVSLGDQASISCRSSQRLVHSNGNTYLHWYLQKTGQSPKLLIYKVSNRFSGVPDRFSGSGSGTDFTLKISRVEAEDLGVYFCSQSTHVPWTFGGGTKLEIK

SEQ ID NO: 297 - protein, 112 aa, Mus musculus
DVVMTQTPLSLPVSLGDQASISCRSSQSLENSNGNTYLNWYLQKPGQSPQLLIYRISTRFSGVLDRFSGSGSGTDFTLKISRVEAEDLGVYFCLQVTHVPYTFGGGTKLEIK

SEQ ID NO: 298 - protein, 112 aa, Mus musculus
DVVMTQTPLSLPVSLGDQASISCRSSQSLIHSNGNTYLYWYLQKPGQSPTLLIYKVSNRFSGVPDRFSGSGSGTDFTLKISRVEAEDLGVYFCSQSTHVPLTFGAGSKLELK

SEQ ID NO: 299 - protein, 112 aa, Mus musculus
DVVMTQTPLSLPVSLGDQASISCRSSQSLVHGNGNTYLHWYLQKPGQSPKLLIYTVSNRFSGVPDRFSGSGSGTDFTLKISRVEAEDLGVYFCSQTIHAPWTFGGGTKLEIK

SEQ ID NO: 300 - protein, 112 aa, Mus musculus
DVVMTQTPLSLPVSLGDQASISCRSSQSLVHSIGNTYLHWYLQKPGQSPKLLIYKVSNRFSGVPDRFSGSGSGTDFTLKIIRVEAEDLGVYFCSQSTHIPWTFGGGTKLELR

SEQ ID NO: 301 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTFKISRVEAENLGVYFCSQSTHVPWTFGGGTKLEIK

SEQ ID NO: 302 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 303 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTLRISRVEADDLGVYFCSQSTHVPYTFGGGTKLEIK

SEQ ID NO: 304 - protein, 112 aa, Mus musculus
DVVMTQTPLSLPVSLGDQASISCRSSQSLVHSNGNTYLHWYLQKPGQSPKLLIYTVSNRFSGVPDRFSGSGSGTDFTLKISRVEAEDLGIYFCSQSTHVPWTFGGGTKLEIK

SEQ ID NO: 305 - protein, 112 aa, Mus musculus
DVVMTQTPLSLPVSLGDQASISCRSSQSLVHSNGNTYLYWYLQKPGQSPKLLIYKVSNRFSGVPDRFSGSGSGTDFTLKISRVEAEDLGVYFCSQSTHVPLTFGAGTKLELK

SEQ ID NO: 306 - protein, 112 aa, Mus musculus
DVVMTQTPLSLSVTIGQPASISCKSSQSLLYTNGKTYLSWLLQRPGQSPKRLIYLVSKLDSGVPDRFTGSGSGTDFTLKISRVEAEDLGAYYCVQGTHFPLTFGGGTKLEIK

SEQ ID NO: 307 - protein, 112 aa, Mus musculus
DVVMTQTPLTLSITIGQPAAVSCKSSQSLLYSNGKTYLNWLLQRPGQSPKRLIYLVSKLDSGVPDRFTGSGSGTDFTLKISRVEAEDLGVYYCVQATHFPLTFGGGTKLEIK

SEQ ID NO: 308 - protein, 113 aa, Mus musculus
DVVMTQTPLTLSVTIGQPASISCKSSQSLLASDGKTYLNWLLQRPGQSPKRLIFLVSKLDSGVPDRFTGSGSGTDFTLKISRVEAEDLGIFYCWQGTHFPTWTFGGGTKLEIK

SEQ ID NO: 309 - protein, 113 aa, Mus musculus
DVVMTQTPLTLSVTIGQPASISCKSSQSLLDSDGKTYLNWLLQRPGQSPKRLIFLVSKLDSGVPDRFTGSGSGTDFTLKISRVEAEDLGFFYCWQGTHFPTWTFGGGTKLEIK

SEQ ID NO: 310 - protein, 113 aa, Mus musculus
DVVMTQTPLTLSVTIGQPASISCKSSQSLLDSDGKTYLNWLLQRPGQSPKRLIFLVSKLDSGVPDRFTGSGSGTDFTLKISRVEAEDLGVFYCWQGTHFPTWTFGGGTKLEIK

SEQ ID NO: 311 - protein, 113 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVFYCWQGTHFPTWTFGGGTKLEIK

SEQ ID NO: 312 - protein, 112 aa, Mus musculus
DVVMTQTPLTLSVTIGQPASISCKSSQSLLYSNGKTYLGWLFQRPGQSPKRLIYLVSTLDSGVPDRFTGSGSGTDFTLKISRVEAEDLGVYYCVQGTHFPLTFGGGTKLEIK

SEQ ID NO: 313 - protein, 112 aa, Mus musculus
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPQTFGGGTKLEIK

SEQ ID NO: 314 - protein, 112 aa, Mus musculus
DVVMTQTPLTLSVTIGQPASISCKSSQSLLYSNGKTYLNWLLQRPGQSPKRLIYLVSTLDSGVPDRFTGSGSGTDFTLKISRVEAEDLGVYYCVQGTHFPLTFGGGTKLEIK

SEQ ID NO: 315 - protein, 112 aa, Mus musculus
DVVMTQTPLTLSVTIGQPASISCKSSQSLLYSNGKTYLSWLFQRPGQSPKRLIYLVSTLDSGVPDRFTGSGSGTDFTLKISRVEAEDLGVYYCVQGTHFPLTFGGGTKLEIK

SEQ ID NO: 316 - protein, 112 aa, Mus musculus
DVVMTQTPLTLSVTIGQPASISCKSSQSLLYSNGKTYLSWLLQRPGQSPKRLIYLVSTLDSGVPDRFTGSGSGTDFTLKISRVEAEDLGVYYCVQGTHFPLTFGGGTKLEIK

SEQ ID NO: 317 - protein, 112 aa, Mus musculus
DVVMTQTPLTLSVTIGQPVSISCKSSQSLLDSDGKTYLNWLLQRPGQSPQRLIYLVSKLDSGVPDRFTGSGSGTDFTLKISRVEAEDLGVYYCWQGTLFPWTFGGGTELEIK

SEQ ID NO: 318 - protein, 111 aa, Mus musculus
DVVMTQTPLTLSVTLGQPASISCKSSQSLLDSDGKTYLNWLLQRPGQSPKRLIYLVSKLDSGVPDRFTGSGSGTDFTLKISRVEAEDLGIYYCWQGTHSWTFGGGTKLEIK

SEQ ID NO: 319 - protein, 112 aa, Mus musculus
DVVVTQTPLSLPVSLGDQASISCRSSQSLVHSNGNTYLYWYLQKPGQSPKLLIYKVSNRFSGVPDRFSGSGSGTDFTLKISRVEAEDLGVYFCSQSTHVPLTFGAGTKLELK

SEQ ID NO: 320 - protein, 108 aa, Mus musculus
EIVLTQSPATLSVTPGDSVSLSCRASQSISNDLHWYQQKSHDSPRLLIKYASQSISGIPSRFSGSGSGTDFTLSINSVETEDFGIYFCQQSHSWPQYTFGGGTKLEIK

SEQ ID NO: 321 - protein, 108 aa, Mus musculus
QIVLTQSPAIMSASLGERVTMTCTASSGVSSTYLHWYQQRPGSSPKLWIYSTSTLASGVPARFSGSGSGTSYSLTISSMEAEDAATYYCHQYHRSPLTFGAGTKLELK

SEQ ID NO: 322 - protein, 108 aa, Mus musculus
QIVLTQSPAIMSASLGERVTMTCTASSSVSSSYLHWYQQKPGSSPKLWVYSTSNLASGVPGRFSGSGSGTSYSLTISSMEAEDAATYYCHHYHRSPPTFGGGTKLEIK

SEQ ID NO: 323 - protein, 108 aa, Mus musculus
QIVLTQSPAIMSASLGERVTMTCTASSSVSSSYLHWYQQKPGSSPKLWVYSTSNLASGVPGRFSGSGSGTSYSLTISSMEAEDAATYYCHHYNRSPPTFGGGTKLEIK

SEQ ID NO: 324 - protein, 108 aa, Mus musculus
QIVLTQSPAIMSASPGKKVTLTCSASSSVSSSYLYWYQQKPGSSPKLWIYSTSNLASGVPARFSGSGSGTSYSLTISSMEAEDAASYFCHQSSSYPPTFGGGTKLEIK

SEQ ID NO: 325 - protein, 108 aa, Mus musculus
QIVLTQSPAIMSASPGKKVTLTCSASSSVSSSYLYWYQQKPGSSPKLWIYSTSNLASGVPARFSGSGSGTSYSLTISSMEAEDAASYFCHQWSSYPPTFGGGTKLEIK

SEQ ID NO: 326 - protein, 108 aa, Mus musculus
QIVLTQSPAIMSASPGKKVTLTCSASSSVSSSYLYWYQQKPGSSPKLWIYSTSNLASGVPARFSGSGSGTSYSLTISSMEAEDAASYFCLQWSSYPPTFGGGTKLEIK

SEQ ID NO: 327 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCQQWSSNPFTFGSGTKLEIK

SEQ ID NO: 328 - protein, 107 aa, Mus musculus
SIVMTQTPKFLLVSAGDRVTITCKASQSMNNGVTWYQQKPGQSPKLLISYASNRYTGVPDRFTGSGYGTDFTFTISAVQAEDLAVYFCQQDYSSPYTFGGGTKLEIK

SEQ ID NO: 329 - protein, 107 aa, Mus musculus
SIVMTQTPKFLLVSAGDRVTITCKASQSVSHGVTWYQQKPGQSPKLLISYASNRYTGVPDRFTGSGYGTDFTFTISTVQAEDLAVYFCQQDYSSPYTFGGGTKLEIK

SEQ ID NO: 330 - protein, 107 aa, Mus musculus
SIVMTQTPKFLLVSAGDRVTITCKASQSVSNAVAWYQQKPGQSPKLLIYYASNRYTGVPDRFTGSGYGTDFTFTISTVQAEDLAVYFCQQDYSSPLTFGAGTKLELK

SEQ ID NO: 331 - protein, 107 aa, Mus musculus
SIVMTQTPKFLLVSAGDRVTITCKASQSVSYGVTWYQQKPGQSPKLLISYSSNRYTGVPDRFTGSGSGTDFTFTISTVQAEDLAVYFCQQDYSSPYTFGGGTKLEIK

SEQ ID NO: 332 - protein, 107 aa, Mus musculus
SIVMTQTPKFLLVSPGDRVTITCRASQSVSNNVAWYQQRPGQSPKLLIYYASNRYTGVPDRFTGSRYGTDFSFTISTVQAEDLAVYFCQQDYSSPYTFGGGTKLEIK

SEQ ID NO: 333 - protein, 8 aa, Mus musculus
GNSFTDYN

SEQ ID NO: 334 - protein, 8 aa, Mus musculus
INPNYGTT

SEQ ID NO: 335 - protein, 6 aa, Mus musculus
AREGNG

SEQ ID NO: 336 - protein, 11 aa, Mus musculus
QSLLDSDGKTY

SEQ ID NO: 337 - skipped

SEQ ID NO: 338 - protein, 9 aa, Mus musculus
WQGTLFPWT

SEQ ID NO: 339 - protein, 8 aa, Mus musculus
GYSFTDYN

SEQ ID NO: 340 - protein, 8 aa, Mus musculus
NNPKYGTT

SEQ ID NO: 341 - protein, 16 aa, Mus musculus
ARRGYYYGSSYGYFDV

SEQ ID NO: 342 - protein, 6 aa, Mus musculus
QDIRSN

SEQ ID NO: 343 - skipped

SEQ ID NO: 344 - protein, 9 aa, Mus musculus
VQYVQFPRT

SEQ ID NO: 345 - protein, 8 aa, Mus musculus
GFTFSDAW

SEQ ID NO: 346 - protein, 8 aa, Mus musculus
IRNKANKH

SEQ ID NO: 347 - protein, 8 aa, Mus musculus
SYDYIFAY

SEQ ID NO: 348 - protein, 11 aa, Mus musculus
QSLVHSNGNTY

SEQ ID NO: 349 - skipped

SEQ ID NO: 350 - protein, 9 aa, Mus musculus
SQSTHVPWT

SEQ ID NO: 351 - protein, 8 aa, Mus musculus
GFNIKGHY

SEQ ID NO: 352 - protein, 8 aa, Mus musculus
IDPDDGAT

SEQ ID NO: 353 - protein, 10 aa, Mus musculus
TRSNPYYFDY

SEQ ID NO: 354 - protein, 6 aa, Mus musculus
QNINVW

SEQ ID NO: 355 - skipped

SEQ ID NO: 356 - protein, 9 aa, Mus musculus
QQGQNYPYT

SEQ ID NO: 357 - protein, 8 aa, Mus musculus
GFNNKNTY

SEQ ID NO: 358 - protein, 8 aa, Mus musculus
IDPANGNV

SEQ ID NO: 359 - protein, 13 aa, Mus musculus
ARGSSRLYYALDY

SEQ ID NO: 360 - protein, 10 aa, Mus musculus
ESVDSYGNSF

SEQ ID NO: 361 - skipped

SEQ ID NO: 362 - protein, 9 aa, Mus musculus
QQSNEDPYT

SEQ ID NO: 363 - protein, 8 aa, Mus musculus
GFTFSDYG

SEQ ID NO: 364 - protein, 8 aa, Mus musculus
ISSGSISS

SEQ ID NO: 365 - protein, 13 aa, Mus musculus
ARHDWRGLYGMDY

SEQ ID NO: 366 - protein, 7 aa, Mus musculus
SSVSSSY

SEQ ID NO: 367 - skipped

SEQ ID NO: 368 - protein, 9 aa, Mus musculus
HQWSSYPPT

SEQ ID NO: 369 - protein, 8 aa, Mus musculus
GFTFSDYY

SEQ ID NO: 370 - protein, 8 aa, Mus musculus
INFDGSGT

SEQ ID NO: 371 - protein, 10 aa, Mus musculus
ARLYDGYYAY

SEQ ID NO: 372 - protein, 9 aa, Mus musculus
SQSTHVPLT

SEQ ID NO: 373 - protein, 8 aa, Mus musculus
INYDGSRT

SEQ ID NO: 374 - protein, 16 aa, Mus musculus
ARNIYYYGGSSYGMDY

SEQ ID NO: 375 - protein, 6 aa, Mus musculus
GNIHNY

SEQ ID NO: 376 - skipped

SEQ ID NO: 377 - protein, 9 aa, Mus musculus
QHFWSTPLT

SEQ ID NO: 378 - protein, 10 aa, Mus musculus
ARLYDGSLDY

SEQ ID NO: 379 - protein, 11 aa, Mus musculus
QSLLYTNGKTY

SEQ ID NO: 380 - protein, 9 aa, Mus musculus
VQGTHFPLT

SEQ ID NO: 381 - protein, 10 aa, Mus musculus
ARLYDGSHDY

SEQ ID NO: 382 - protein, 11 aa, Mus musculus
QSLLYSNGKTY

SEQ ID NO: 383 - protein, 9 aa, Mus musculus
VQGTHFPQT

SEQ ID NO: 384 - protein, 8 aa, Mus musculus
INFDGSSS

SEQ ID NO: 385 - protein, 11 aa, Mus musculus
QSLIHSNGNTY

SEQ ID NO: 386 - protein, 9 aa, Mus musculus
VQATHFPLT

SEQ ID NO: 387 - protein, 8 aa, Mus musculus
GYTFTDYN

SEQ ID NO: 388 - protein, 8 aa, Mus musculus
INPNNGGI

SEQ ID NO: 389 - protein, 6 aa, Mus musculus
VGSLDY

SEQ ID NO: 390 - protein, 6 aa, Mus musculus
QDINNY

SEQ ID NO: 391 - skipped

SEQ ID NO: 392 - protein, 9 aa, Mus musculus
QQGYMLPLT

SEQ ID NO: 393 - protein, 8 aa, Mus musculus
EYTFADYY

SEQ ID NO: 394 - protein, 8 aa, Mus musculus
INPYNGGA

SEQ ID NO: 395 - protein, 18 aa, Mus musculus
ARDADFYGNSYVGYAMDY

SEQ ID NO: 396 - protein, 6 aa, Mus musculus
QSINTD

SEQ ID NO: 397 - skipped

SEQ ID NO: 398 - protein, 10 aa, Mus musculus
QQSHTWPQYT

SEQ ID NO: 399 - protein, 8 aa, Mus musculus
GYTFTSYW

SEQ ID NO: 400 - protein, 8 aa, Mus musculus
IDPNSGGT

SEQ ID NO: 401 - protein, 13 aa, Mus musculus
TKDGNTPYYAMDY

SEQ ID NO: 402 - protein, 6 aa, Mus musculus
QSVSNA

SEQ ID NO: 403 - protein, 9 aa, Mus musculus
QQDYSSPLT

SEQ ID NO: 404 - protein, 8 aa, Mus musculus
DFNIKDYY

SEQ ID NO: 405 - protein, 8 aa, Mus musculus
IDPDDGET

SEQ ID NO: 406 - protein, 10 aa, Mus musculus
SRSNPYYFDY

SEQ ID NO: 407 - protein, 8 aa, Mus musculus
IDPEDDET

SEQ ID NO: 408 - protein, 10 aa, Mus musculus
ARSNPYYFDY

SEQ ID NO: 409 - protein, 8 aa, Mus musculus
GFNIKDFY

SEQ ID NO: 410 - protein, 8 aa, Mus musculus
IDPEDGKT

SEQ ID NO: 411 - protein, 10 aa, Mus musculus
ARSNPYFFDY

SEQ ID NO: 412 - protein, 6 aa, Mus musculus
QTINVW

SEQ ID NO: 413 - protein, 9 aa, Mus musculus
HQGQSYPYT

SEQ ID NO: 414 - protein, 8 aa, Mus musculus
GFNIKDYY

SEQ ID NO: 415 - protein, 8 aa, Mus musculus
IDPDNGET

SEQ ID NO: 416 - protein, 10 aa, Mus musculus
ARSSPYYFDY

SEQ ID NO: 417 - protein, 9 aa, Mus musculus
QQGQNFPYT

SEQ ID NO: 418 - protein, 9 aa, Mus musculus
QQGQSYPYT

SEQ ID NO: 419 - protein, 8 aa, Mus musculus
IDPEDGET

SEQ ID NO: 420 - protein, 10 aa, Mus musculus
GRSNPYYFDY

SEQ ID NO: 421 - protein, 6 aa, Mus musculus
QNVGTN

SEQ ID NO: 422 - skipped

SEQ ID NO: 423 - protein, 9 aa, Mus musculus
QQYNTYPLT

SEQ ID NO: 424 - protein, 8 aa, Mus musculus
IDPDNDKI

SEQ ID NO: 425 - protein, 6 aa, Mus musculus
QTINIW

SEQ ID NO: 426 - protein, 8 aa, Mus musculus
IDPENDKI

SEQ ID NO: 427 - protein, 8 aa, Mus musculus
IDPDNDEI

SEQ ID NO: 428 - protein, 9 aa, Mus musculus
QQGQNIPYT

SEQ ID NO: 429 - protein, 8 aa, Mus musculus
IDPDDAET

SEQ ID NO: 430 - protein, 12 aa, Mus musculus
TRDFGYTDYFDY

SEQ ID NO: 431 - protein, 6 aa, Mus musculus
QDVSTA

SEQ ID NO: 432 - protein, 8 aa, Mus musculus
QQHYSTPT

SEQ ID NO: 433 - protein, 8 aa, Mus musculus
IYPYNGGT

SEQ ID NO: 434 - protein, 13 aa, Mus musculus
AREDYGSREWFAY

SEQ ID NO: 435 - protein, 8 aa, Mus musculus
GYTFTDYY

SEQ ID NO: 436 - protein, 8 aa, Mus musculus
INLNNGGT

SEQ ID NO: 437 - protein, 6 aa, Mus musculus
EGLGGY

SEQ ID NO: 438 - protein, 11 aa, Mus musculus
QSIVHSNGNTY

SEQ ID NO: 439 - skipped

SEQ ID NO: 440 - protein, 9 aa, Mus musculus
FQGSHVPFT

SEQ ID NO: 441 - protein, 8 aa, Mus musculus
GYTLTDYY

SEQ ID NO: 442 - protein, 8 aa, Mus musculus
INPNNGDI

SEQ ID NO: 443 - protein, 11 aa, Mus musculus
QTIVHSNGNTY

SEQ ID NO: 444 - protein, 8 aa, Mus musculus
GNTFTAFY

SEQ ID NO: 445 - protein, 8 aa, Mus musculus
VNPYNGGA

SEQ ID NO: 446 - protein, 7 aa, Mus musculus
LLWEFAH

SEQ ID NO: 447 - protein, 8 aa, Mus musculus
WQGTHSWT

SEQ ID NO: 448 - protein, 8 aa, Mus musculus
EYTFTDYY

SEQ ID NO: 449 - protein, 18 aa, Mus musculus
ARDVDFYGNRYVGYAMDY

SEQ ID NO: 450 - protein, 6 aa, Mus musculus
QSISNN

SEQ ID NO: 451 - protein, 10 aa, Mus musculus
QQSNSWPQYT

SEQ ID NO: 452 - protein, 8 aa, Mus musculus
GYSFTGYH

SEQ ID NO: 453 - protein, 8 aa, Mus musculus
INPYNSAS

SEQ ID NO: 454 - protein, 11 aa, Mus musculus
VRGAYGNPLDY

SEQ ID NO: 455 - protein, 6 aa, Mus musculus
QDINSY

SEQ ID NO: 456 - skipped

SEQ ID NO: 457 - protein, 9 aa, Mus musculus
LQYDEYPYT

SEQ ID NO: 458 - protein, 8 aa, Mus musculus
INPYNSAT

SEQ ID NO: 459 - skipped

SEQ ID NO: 460 - protein, 8 aa, Mus musculus
GYTFTDYH

SEQ ID NO: 461 - protein, 8 aa, Mus musculus
ISPYNGGT

SEQ ID NO: 462 - protein, 11 aa, Mus musculus
ARSDYGSPLDY

SEQ ID NO: 463 - protein, 9 aa, Mus musculus
LQYDEFPYT

SEQ ID NO: 464 - protein, 18 aa, Mus musculus
ARDVDFYDNAYVGYAMDY

SEQ ID NO: 465 - protein, 6 aa, Mus musculus
QTISNN

SEQ ID NO: 466 - protein, 8 aa, Mus musculus
INPYNGGT

SEQ ID NO: 467 - protein, 15 aa, Mus musculus
ARGGDYDVNYSAMDY

SEQ ID NO: 468 - protein, 10 aa, Mus musculus
WQGTHFPTWT

SEQ ID NO: 469 - protein, 8 aa, Mus musculus
INPYNGGV

SEQ ID NO: 470 - protein, 18 aa, Mus musculus
ARDADFHGNSYVGYAMDY

SEQ ID NO: 471 - protein, 6 aa, Mus musculus
QSISND

SEQ ID NO: 472 - protein, 10 aa, Mus musculus
QQSHSWPQYT

SEQ ID NO: 473 - protein, 8 aa, Mus musculus
INPYNSGI

SEQ ID NO: 474 - protein, 18 aa, Mus musculus
ARDVDYFDNKYVGYAMDY

SEQ ID NO: 475 - protein, 8 aa, Mus musculus
GYTFTRYW

SEQ ID NO: 476 - protein, 8 aa, Mus musculus
IYPGNSDT

SEQ ID NO: 477 - protein, 10 aa, Mus musculus
TFYYGNYVNY

SEQ ID NO: 478 - protein, 8 aa, Mus musculus
GYTFIRYW

SEQ ID NO: 479 - protein, 10 aa, Mus musculus
TFYYGNYVNF

SEQ ID NO: 480 - protein, 10 aa, Mus musculus
TFFYGNYVNY

SEQ ID NO: 481 - protein, 12 aa, Mus musculus
QSLLYSSNQKNY

SEQ ID NO: 482 - skipped

SEQ ID NO: 483 - protein, 9 aa, Mus musculus
QQYYSYPRT

SEQ ID NO: 484 - protein, 11 aa, Mus musculus
QSLLASDGKTY

SEQ ID NO: 485 - protein, 9 aa, Mus musculus
QQYNSYPYT

SEQ ID NO: 486 - protein, 8 aa, Mus musculus
ISSGGSYT

SEQ ID NO: 487 - protein, 17 aa, Mus musculus
TRHEITTVLSTDYAMDY

SEQ ID NO: 488 - protein, 11 aa, Mus musculus
QSLVHSIGNTY

SEQ ID NO: 489 - protein, 9 aa, Mus musculus
SQSTHIPWT

SEQ ID NO: 490 - protein, 13 aa, Mus musculus
ARHDWRGLYAMDY

SEQ ID NO: 491 - protein, 9 aa, Mus musculus
HQSSSYPPT

SEQ ID NO: 492 - protein, 8 aa, Mus musculus
ISGGSISS

SEQ ID NO: 493 - protein, 9 aa, Mus musculus
LQWSSYPPT

SEQ ID NO: 494 - protein, 8 aa, Mus musculus
GFTFSGFW

SEQ ID NO: 495 - protein, 8 aa, Mus musculus
INSDGSTI

SEQ ID NO: 496 - protein, 7 aa, Mus musculus
MSLLLNV

SEQ ID NO: 497 - protein, 6 aa, Mus musculus
QEISGY

SEQ ID NO: 498 - protein, 9 aa, Mus musculus
LQYANYPLT

SEQ ID NO: 499 - protein, 8 aa, Mus musculus
INYDGTNT

SEQ ID NO: 500 - protein, 15 aa, Mus musculus
GRGIYYYGRSHAMDY

SEQ ID NO: 501 - protein, 6 aa, Mus musculus
QGINNN

SEQ ID NO: 502 - protein, 9 aa, Mus musculus
VQYDQFPPT

SEQ ID NO: 503 - protein, 8 aa, Mus musculus
IDPSDNYT

SEQ ID NO: 504 - protein, 12 aa, Mus musculus
AREGAFSSPFAY

SEQ ID NO: 505 - protein, 12 aa, Mus musculus
AKEGAFSSPFAY

SEQ ID NO: 506 - protein, 9 aa, Mus musculus
VQYIQFPRT

SEQ ID NO: 507 - protein, 8 aa, Mus musculus
IYPGSDNT

SEQ ID NO: 508 - protein, 11 aa, Mus musculus
ARSGNYYAMDY

SEQ ID NO: 509 - protein, 6 aa, Mus musculus
QSVSNN

SEQ ID NO: 510 - protein, 9 aa, Mus musculus
QQDYSSPYT

SEQ ID NO: 511 - protein, 8 aa, Mus musculus
IYPGSGNT

SEQ ID NO: 512 - protein, 10 aa, Mus musculus
ARAYDYGFPY

SEQ ID NO: 513 - protein, 6 aa, Mus musculus
QSMNNG

SEQ ID NO: 514 - protein, 10 aa, Mus musculus
ARAYDYGFAY

SEQ ID NO: 515 - protein, 6 aa, Mus musculus
QSVSYG

SEQ ID NO: 516 - skipped

SEQ ID NO: 517 - protein, 6 aa, Mus musculus
QSVSHG

SEQ ID NO: 518 - protein, 8 aa, Mus musculus
GYIFRTYG

SEQ ID NO: 519 - protein, 8 aa, Mus musculus
INTYSGEP

SEQ ID NO: 520 - protein, 9 aa, Mus musculus
ARAQHYFDW

SEQ ID NO: 521 - protein, 6 aa, Mus musculus
QDIDSY

SEQ ID NO: 522 - skipped

SEQ ID NO: 523 - protein, 8 aa, Mus musculus
GYTFRTYG

SEQ ID NO: 524 - protein, 6 aa, Mus musculus
QDIDRY

SEQ ID NO: 525 - protein, 8 aa, Mus musculus
IYPGNGNT

SEQ ID NO: 526 - protein, 16 aa, Mus musculus
ARGGSSDRIYYYAMDY

SEQ ID NO: 527 - protein, 9 aa, Mus musculus
QQGQSYPLT

SEQ ID NO: 528 - protein, 8 aa, Mus musculus
GYTFTNFW

SEQ ID NO: 529 - protein, 8 aa, Mus musculus
IDPSDIST

SEQ ID NO: 530 - protein, 12 aa, Mus musculus
ARNDYDGGYFDY

SEQ ID NO: 531 - protein, 11 aa, Mus musculus
QSLVHGNGNTY

SEQ ID NO: 532 - skipped

SEQ ID NO: 533 - protein, 9 aa, Mus musculus
SQTIHAPWT

SEQ ID NO: 534 - protein, 8 aa, Mus musculus
GFSLTSSV

SEQ ID NO: 535 - protein, 7 aa, Mus musculus
IWAGGDT

SEQ ID NO: 536 - protein, 9 aa, Mus musculus
ARNLNAMDY

SEQ ID NO: 537 - protein, 9 aa, Mus musculus
FQGSHVPWT

SEQ ID NO: 538 - protein, 8 aa, Mus musculus
GYTFTDFY

SEQ ID NO: 539 - protein, 8 aa, Mus musculus
IYPGGANT

SEQ ID NO: 540 - protein, 10 aa, Mus musculus
ARGANWAFAS

SEQ ID NO: 541 - protein, 6 aa, Mus musculus
QGVSTA

SEQ ID NO: 542 - protein, 9 aa, Mus musculus
QQYYNTPYT

SEQ ID NO: 543 - protein, 8 aa, Mus musculus
GYILTDYY

SEQ ID NO: 544 - protein, 16 aa, Mus musculus
ARGGSSDRIYYYAMDH

SEQ ID NO: 545 - protein, 8 aa, Mus musculus
GYTLIDYY

SEQ ID NO: 546 - protein, 16 aa, Mus musculus
ARGGSSDRIYYFAMDY

SEQ ID NO: 547 - protein, 8 aa, Mus musculus
GYTLNDYY

SEQ ID NO: 548 - protein, 8 aa, Mus musculus
IYPGNNNT

SEQ ID NO: 549 - protein, 16 aa, Mus musculus
ARGGSSVRVYYYAMDY

SEQ ID NO: 550 - protein, 16 aa, Mus musculus
ARGGSSVRVYYYAMDF

SEQ ID NO: 551 - protein, 8 aa, Mus musculus
GFSLISYG

SEQ ID NO: 552 - protein, 8 aa, Mus musculus
IWSGGNTD

SEQ ID NO: 553 - protein, 9 aa, Mus musculus
ARTANALDY

SEQ ID NO: 554 - protein, 8 aa, Mus musculus
GYTFTNYW

SEQ ID NO: 555 - protein, 12 aa, Mus musculus
AGEGAFSSPFAY

SEQ ID NO: 556 - protein, 8 aa, Mus musculus
GNTFTSYW

SEQ ID NO: 557 - protein, 8 aa, Mus musculus
IDPSDSYI

SEQ ID NO: 558 - protein, 12 aa, Mus musculus
AREGAYSSPFAY

SEQ ID NO: 559 - skipped

SEQ ID NO: 560 - protein, 8 aa, Mus musculus
IDPDSGGT

SEQ ID NO: 561 - protein, 9 aa, Mus musculus
ARYYWFFDI

SEQ ID NO: 562 - protein, 9 aa, Mus musculus
QQHFNTPFT

SEQ ID NO: 563 - protein, 8 aa, Mus musculus
CYTFISYW

SEQ ID NO: 564 - protein, 6 aa, Mus musculus
QDVGAA

SEQ ID NO: 565 - protein, 8 aa, Mus musculus
GYSFISSW

SEQ ID NO: 566 - protein, 8 aa, Mus musculus
IHPSDSDT

SEQ ID NO: 567 - protein, 13 aa, Mus musculus
AIIHRGFYFAMDY

SEQ ID NO: 568 - protein, 8 aa, Mus musculus
GYTFISYW

SEQ ID NO: 569 - protein, 8 aa, Mus musculus
IDPNSGDT

SEQ ID NO: 570 - protein, 10 aa, Mus musculus
ARWGWDAFAY

SEQ ID NO: 571 - protein, 11 aa, Mus musculus
QSLENSDGNTY

SEQ ID NO: 572 - skipped

SEQ ID NO: 573 - protein, 9 aa, Mus musculus
LQVTHVPPT

SEQ ID NO: 574 - protein, 8 aa, Mus musculus
GYSFISYW

SEQ ID NO: 575 - protein, 13 aa, Mus musculus
TISHYSGSSPETY

SEQ ID NO: 576 - protein, 9 aa, Mus musculus
FQGSHVPYT

SEQ ID NO: 577 - protein, 8 aa, Mus musculus
GYTFISHW

SEQ ID NO: 578 - protein, 13 aa, Mus musculus
AIWSYYYGSRLDY

SEQ ID NO: 579 - protein, 11 aa, Mus musculus
QNIVHSNGNTY

SEQ ID NO: 580 - protein, 8 aa, Mus musculus
QGSHVPYT

SEQ ID NO: 581 - protein, 8 aa, Mus musculus
GYTFTTFW

SEQ ID NO: 582 - protein, 8 aa, Mus musculus
IDPSDSYT

SEQ ID NO: 583 - protein, 14 aa, Mus musculus
ARNYGSNYVGYFDY

SEQ ID NO: 584 - protein, 6 aa, Mus musculus
QDISNY

SEQ ID NO: 585 - protein, 9 aa, Mus musculus
QQANALPWT

SEQ ID NO: 586 - protein, 8 aa, Mus musculus
GYTFNTYW

SEQ ID NO: 587 - protein, 9 aa, Mus musculus
QQAKALPWT

SEQ ID NO: 588 - protein, 8 aa, Mus musculus
GYTFSTYW

SEQ ID NO: 589 - protein, 14 aa, Mus musculus
ARNYGSNYVGYFDH

SEQ ID NO: 590 - protein, 9 aa, Mus musculus
QQGNALPWT

SEQ ID NO: 591 - protein, 6 aa, Mus musculus
QGISNY

SEQ ID NO: 592 - protein, 9 aa, Mus musculus
QQGNSLPWT

SEQ ID NO: 593 - protein, 8 aa, Mus musculus
IDPSDSYS

SEQ ID NO: 594 - protein, 9 aa, Mus musculus
QQGNTLPWT

SEQ ID NO: 595 - protein, 8 aa, Mus musculus
GYTFTTYW

SEQ ID NO: 596 - protein, 8 aa, Mus musculus
IDPSDGYS

SEQ ID NO: 597 - protein, 9 aa, Mus musculus
QQGKALPWT

SEQ ID NO: 598 - protein, 8 aa, Mus musculus
IDPSDGYT

SEQ ID NO: 599 - protein, 6 aa, Mus musculus
QDITNY

SEQ ID NO: 600 - skipped

SEQ ID NO: 601 - protein, 8 aa, Mus musculus
GYNFTNYW

SEQ ID NO: 602 - protein, 8 aa, Mus musculus
IYPSDNKT

SEQ ID NO: 603 - protein, 6 aa, Mus musculus
AASMDY

SEQ ID NO: 604 - protein, 12 aa, Mus musculus
QSLLNSKNQKNF

SEQ ID NO: 605 - protein, 9 aa, Mus musculus
QNDYSYPYT

SEQ ID NO: 606 - protein, 8 aa, Mus musculus
GYNFTSYW

SEQ ID NO: 607 - protein, 8 aa, Mus musculus
IYPSDSKT

SEQ ID NO: 608 - protein, 6 aa, Mus musculus
ATSMDY

SEQ ID NO: 609 - protein, 9 aa, Mus musculus
QNDYDYPYT

SEQ ID NO: 610 - protein, 8 aa, Mus musculus
GYTFINYW

SEQ ID NO: 611 - protein, 8 aa, Mus musculus
IDPSHTST

SEQ ID NO: 612 - protein, 9 aa, Mus musculus
SQTTHVPWT

SEQ ID NO: 613 - protein, 8 aa, Mus musculus
GYTFSRYW

SEQ ID NO: 614 - protein, 8 aa, Mus musculus
IDPSDNNT

SEQ ID NO: 615 - protein, 8 aa, Mus musculus
IDPSSSST

SEQ ID NO: 616 - protein, 11 aa, Mus musculus
QSLVHSNGITY

SEQ ID NO: 617 - protein, 8 aa, Mus musculus
IDPSDSST

SEQ ID NO: 618 - protein, 11 aa, Mus musculus
QSLLHSNGNTY

SEQ ID NO: 619 - protein, 8 aa, Mus musculus
IDPSDSNT

SEQ ID NO: 620 - protein, 12 aa, Mus musculus
SRNDYDGGYFDY

SEQ ID NO: 621 - protein, 8 aa, Mus musculus
GYSFTIYW

SEQ ID NO: 622 - protein, 8 aa, Mus musculus
IDPSTSNT

SEQ ID NO: 623 - protein, 11 aa, Mus musculus
QRLVHSNGNTY

SEQ ID NO: 624 - protein, 8 aa, Mus musculus
IDPSDTNT

SEQ ID NO: 625 - protein, 8 aa, Mus musculus
IDPSDSYR

SEQ ID NO: 626 - protein, 9 aa, Mus musculus
QQASTLPWT

SEQ ID NO: 627 - protein, 12 aa, Mus musculus
AREGAFSSPFAF

SEQ ID NO: 628 - protein, 12 aa, Mus musculus
AKEGAYSSPFAY

SEQ ID NO: 629 - protein, 12 aa, Mus musculus
VREGAFSSPFAY

SEQ ID NO: 630 - protein, 8 aa, Mus musculus
GYSFISHW

SEQ ID NO: 631 - protein, 13 aa, Mus musculus
AIWSYYYGTRLDY

SEQ ID NO: 632 - protein, 12 aa, Mus musculus
ARNDYDGGYFDF

SEQ ID NO: 633 - protein, 11 aa, Mus musculus
QSLVHSNGHTY

SEQ ID NO: 634 - protein, 8 aa, Mus musculus
GYTFISYC

SEQ ID NO: 635 - protein, 8 aa, Mus musculus
IDPNNGGP

SEQ ID NO: 636 - protein, 10 aa, Mus musculus
ARWGWDGFAY

SEQ ID NO: 637 - protein, 11 aa, Mus musculus
QSLENSNGNTY

SEQ ID NO: 638 - protein, 9 aa, Mus musculus
LQVTHVPYT

SEQ ID NO: 639 - protein, 8 aa, Mus musculus
IDPNNGGT

SEQ ID NO: 640 - protein, 11 aa, Mus musculus
QSLENSYGNTY

SEQ ID NO: 641 - protein, 10 aa, Mus musculus
TRWGWDGFAY

SEQ ID NO: 642 - skipped

SEQ ID NO: 643 - protein, 8 aa, Mus musculus
IDPNSGIT

SEQ ID NO: 644 - protein, 8 aa, Mus musculus
IDPNSGTT

SEQ ID NO: 645 - protein, 8 aa, Mus musculus
IDPNYGGP

SEQ ID NO: 646 - protein, 9 aa, Mus musculus
LQITHVPYT

SEQ ID NO: 647 - protein, 9 aa, Mus musculus
LQLTHVPYT

SEQ ID NO: 648 - protein, 5 aa, Mus musculus
SSVSY

SEQ ID NO: 649 - skipped

SEQ ID NO: 650 - protein, 9 aa, Mus musculus
QQWSSNPFT

SEQ ID NO: 651 - protein, 8 aa, Mus musculus
IDPNYGGS

SEQ ID NO: 652 - protein, 6 aa, Mus musculus
QDIGSS

SEQ ID NO: 653 - skipped

SEQ ID NO: 654 - protein, 9 aa, Mus musculus
LQYASSPFT

SEQ ID NO: 655 - protein, 8 aa, Mus musculus
INPNNGGT

SEQ ID NO: 656 - protein, 8 aa, Mus musculus
GYTFVSYW

SEQ ID NO: 657 - protein, 8 aa, Mus musculus
IDPNNGGS

SEQ ID NO: 658 - protein, 8 aa, Mus musculus
IDPSDSYA

SEQ ID NO: 659 - protein, 9 aa, Mus musculus
QQEKTLPWT

SEQ ID NO: 660 - protein, 8 aa, Mus musculus
GYTFRSYG

SEQ ID NO: 661 - protein, 8 aa, Mus musculus
IYPRSGNT

SEQ ID NO: 662 - protein, 13 aa, Mus musculus
ARSTAVVAGGFDY

SEQ ID NO: 663 - protein, 12 aa, Mus musculus
QSLFNSRTQKNS

SEQ ID NO: 664 - protein, 9 aa, Mus musculus
QNDYTYPWT

SEQ ID NO: 665 - protein, 8 aa, Mus musculus
GYTFTNYG

SEQ ID NO: 666 - protein, 8 aa, Mus musculus
IYPRSGNS

SEQ ID NO: 667 - protein, 13 aa, Mus musculus
ARSTAIVAGGFDY

SEQ ID NO: 668 - protein, 12 aa, Mus musculus
QSLLNSRIQKNS

SEQ ID NO: 669 - protein, 8 aa, Mus musculus
GYTFTSYG

SEQ ID NO: 670 - protein, 14 aa, Mus musculus
ARSTYGSSSYAMDY

SEQ ID NO: 671 - protein, 8 aa, Mus musculus
GYTFPDYE

SEQ ID NO: 672 - protein, 8 aa, Mus musculus
IDPETGGT

SEQ ID NO: 673 - protein, 5 aa, Mus musculus
TRWAY

SEQ ID NO: 674 - protein, 9 aa, Mus musculus
HHYHRSPPT

SEQ ID NO: 675 - protein, 8 aa, Mus musculus
GYSITGYY

SEQ ID NO: 676 - protein, 8 aa, Mus musculus
IYPGSGSI

SEQ ID NO: 677 - protein, 12 aa, Mus musculus
AREAWGNWYFDV

SEQ ID NO: 678 - protein, 6 aa, Mus musculus
QSIGTS

SEQ ID NO: 679 - protein, 9 aa, Mus musculus
QQSNSWPYT

SEQ ID NO: 680 - protein, 8 aa, Mus musculus
GYTFSDYE

SEQ ID NO: 681 - protein, 8 aa, Mus musculus
IDPETGDT

SEQ ID NO: 682 - protein, 8 aa, Mus musculus
TSNGYPEH

SEQ ID NO: 683 - protein, 7 aa, Mus musculus
SGVSSTY

SEQ ID NO: 684 - protein, 9 aa, Mus musculus
HQYHRSPLT

SEQ ID NO: 685 - protein, 8 aa, Mus musculus
GYTFTDFE

SEQ ID NO: 686 - protein, 15 aa, Mus musculus
TRGIYYGNYGSAMDY

SEQ ID NO: 687 - protein, 9 aa, Mus musculus
FQGSHAPLT

SEQ ID NO: 688 - protein, 8 aa, Mus musculus
GYTFTDYE

SEQ ID NO: 689 - protein, 5 aa, Mus musculus
TRWAF

SEQ ID NO: 690 - protein, 9 aa, Mus musculus
SQSTHVPYT

SEQ ID NO: 691 - protein, 9 aa, Mus musculus
HHYNRSPPT

SEQ ID NO: 692 - protein, 8 aa, Mus musculus
GYAFSNYL

SEQ ID NO: 693 - protein, 8 aa, Mus musculus
INPGNGGT

SEQ ID NO: 694 - protein, 11 aa, Mus musculus
ARTAYGSPLDY

SEQ ID NO: 695 - protein, 6 aa, Mus musculus
QDINRY

SEQ ID NO: 696 - protein, 8 aa, Mus musculus
GYAFTNYL

SEQ ID NO: 697 - protein, 8 aa, Mus musculus
INPGSGAT

SEQ ID NO: 698-699 - skipped (2 entries)

SEQ ID NO: 700 - protein, 14 aa, Mus musculus
ARRTTVVAPYAMDY

SEQ ID NO: 701 - protein, 9 aa, Mus musculus
QQGNTLPRT

SEQ ID NO: 702 - protein, 8 aa, Mus musculus
ISPGSGAT

SEQ ID NO: 703 - protein, 11 aa, Mus musculus
ARSAYGSPLDF

SEQ ID NO: 704 - skipped

SEQ ID NO: 705 - protein, 9 aa, Mus musculus
QQYDEFPYT

SEQ ID NO: 706 - protein, 6 aa, Mus musculus
QNIGTS

SEQ ID NO: 707 - protein, 9 aa, Mus musculus
QQSNNWPFT

SEQ ID NO: 708 - protein, 8 aa, Mus musculus
ISPGSGGT

SEQ ID NO: 709 - protein, 11 aa, Mus musculus
ARSAYGHVFDY

SEQ ID NO: 710 - protein, 8 aa, Mus musculus
GYTFRSNG

SEQ ID NO: 711 - protein, 8 aa, Mus musculus
IYPRSGNI

SEQ ID NO: 712 - protein, 13 aa, Mus musculus
ARSTTVVAGGFDF

SEQ ID NO: 713 - protein, 12 aa, Mus musculus
QSLLNSRTQKNS

SEQ ID NO: 714 - protein, 8 aa, Mus musculus
GYSFTGYY

SEQ ID NO: 715 - protein, 9 aa, Mus musculus
GYSFTGYYI

SEQ ID NO: 716 - protein, 8 aa, Mus musculus
IGWIYPGS

SEQ ID NO: 717 - protein, 8 aa, Mus musculus
GYTFTSFG

SEQ ID NO: 718 - skipped

SEQ ID NO: 719 - protein, 9 aa, Mus musculus
QNDYNYPWT

SEQ ID NO: 720 - protein, 6 aa, Mus musculus
DHINNW

SEQ ID NO: 721 - skipped

SEQ ID NO: 722 - protein, 9 aa, Mus musculus
QQYWNTPYT